(19) United States
(12) Patent Application Publication
(10) Pub. No.: US 2025/0125004 A1
(43) Pub. Date: Apr. 17, 2025

(54) SYSTEMS AND METHODS FOR POST-TRANSLATIONAL MODIFICATION-INSPIRED DRUG DESIGN AND SCREENING

(71) Applicant: Ensem Therapeutics, Inc., Waltham, MA (US)

(72) Inventors: Zhongjie Liang, Waltham, MA (US); Cheng Luo, Waltham, MA (US); Minghong Hao, Quincy, MA (US)

(73) Assignee: Ensem Therapeutics, Inc., Waltham, MA (US)

(21) Appl. No.: 18/684,483

(22) PCT Filed: Aug. 25, 2022
(86) PCT No.: PCT/CN2022/114966

§ 371 Date: Feb. 16, 2024

(30) Foreign Application Priority Data
Aug. 25, 2021 (WO) PCT/CN2021/114523

(51) Int. Cl. G16B 15/30; G16B 15/20; G16B 40/30

(52) U.S. Cl. CPC G16B 15/30; G16B 15/20; G16B 40/30

(57) ABSTRACT

Novel systems and methods for drug design and screening exploiting dynamics of protein post-translational modifications are provided.

### CROSS REFERENCE TO RELATED APPLICATIONS

This application claims the benefit of and priority to International Application No. PCT/CN2021/114523 filed Aug. 25, 2021, the entire disclosure of which is hereby incorporated by reference in its entirety for all purposes.

### TECHNICAL FIELDS OF THE INVENTION

The invention generally relates to design, identification, and testing of compounds, primarily using in silico methods, for therapeutic applications. More particularly, the invention provides novel systems and methods for drug design and screening exploiting dynamics of protein post-translational modifications.

### BACKGROUND OF THE INVENTION

The predominant existence of covalent modifications of proteins by post-translational modification (PTM) enzymes contributes to the diversity of protein functions, involving greater than 670 modification types on approximately 900,000 PTM sites (http://www.uniprot.org/docs/ptmlist.txt). The high dynamic process of PTMs within a cell forms a complex and ever-changing nexus of protein modifications, which plays central roles in various cellular signaling functions through different mechanisms, including regulating protein-protein interactions, protein localizations, degradations, cleavages, or allosterically regulating enzyme activities¹. Recently, the database manually collected 1,950 known PTM-disease associations in 749 proteins from the literature, including 23 types of PTMs and 275 types of diseases². Accumulating evidence has shown that the abnormal status of PTMs is frequently involved in various human diseases, such as cancers, diabetes, and neurodegenerative diseases, making PTMs valuable for biomarker studies and personalized therapies.

To understand PTM functions, extensive effort has been devoted to data compilation for mapping the PTM information onto protein structures³. Several databases, including Phospho3D⁴, TopPTM⁵, PTM-SD^{3d}, PhosphoSitePlus^{3c}, and comprehensive dbPTM^{3a}, compiled the PTM sites within protein three-dimensional structures and explored PTM-disease associations. Through mapping phosphorylation sites onto 453 non-redundant structures of soluble mammalian target proteins bound to inhibitors, 29% of them have been identified with phosphorylation sites located within 12 Å of a small molecule binding site⁶. Using large-scale screening for PTM sites and drug binding sites in the Protein Data Bank (PDB), 3,951 PTM sites located on or within 12 Å of drug-target binding sites have been curated and archived in the CruxPTM database⁷. The structural correlations between PTM sites and drug-protein binding sites have therefore enhanced understanding of the enlarged targetable space and biological mechanisms associated with PTMs.

Regarding structural rearrangements with the introduction of PTMs, a few studies have systematically characterized the role of PTMs as conformational switches. Through statistical analyses of root-mean-square deviations between modified and unmodified structures of the same protein, it was discovered that N-glycosylation and phosphorylation induced significant yet not extreme changes to protein structures⁸. The percentage of large conformational changes was unexpectedly small; only 7% of the glycosylated and 13% of phosphorylated proteins underwent global changes >2 Å. Using structural alphabet protein blocks, the backbone conformations of modified residues within protein structures indicated that PTMs could either stabilize or destabilize the backbone structure, at either a local or global scale, depending on the PTM types⁹. However, in the exploration of the links between the structural rearrangements introduced by PTMs and protein functions, the molecular effects of PTMs on protein dynamics remain poorly understood. Molecular modeling of PTMs combined with molecular dynamics simulation is a viable alternative. Some recent computational studies have investigated the effect of PTMs on the stability of specific proteins¹⁰, but the growing success of these kinds of simulations also relies on the increasing amount of experimental data and the development of accurate PTM force field parameter data.

PTMs have currently been shown to affect enzyme function and drug binding affinity in two ways: (i) directly (or orthosterically), via direct effects on ligand binding sites by adjacent PTMs; and (ii) allosterically, via conformational changes induced from the distant PTM sites¹¹. The dynamics PTM code has been proposed, in which PTMs lead to conformational and dynamics changes by accommodating the structural environment with the introduction of PTM perturbations¹¹. PTMs have thus enriched the proteome complexity to a great extent with little evolutionary cost, and clearly constitute a potential unexplored target space. In drug design, targeting active PTM isoforms will not only largely extends the proteome space, but also enables rational design to develop PTM protein isoform specific drugs toward precision medicine.

Despite recent progress regarding the potential and future directions in drug design targeting the active PTM protein isoforms¹², effective and practical strategies have remained elusive in part due to the difficulties posed by the functional diversity of PTM isoforms and the dynamics induced by PTMs.

### SUMMARY OF THE INVENTION

The invention provides novel in silico-based systems and methods for drug design and screening exploiting dynamics of protein PTMs. An integrated framework incorporating sequence, structural topology and dynamics features with protein modeling and machining learning is disclosed, which allows efficient characterization of functionalities and accurate classification of druggabilities of PTMs. Along with molecular docking techniques, the PTM inspired drug design and screening approach offers unprecedented capability and efficiency for identification of novel pharmacophores and drug candidates.

A central feature of the PTM inspired drug design and screening system and method disclosed herein is that it takes into considerations the functional diversity of PTM isoforms and the dynamics induced by PTMs, in conjunction with machine learning models and in silico docketing techniques, to achieve superior results in both identification of potentially druggable pocket induced by, selected by and/or associated with PTM site and finding pharmacophores or compounds exhibiting desired levels of interaction with such PTM sites.

PTM on protein is an essential mechanism to generate various structural isoforms, which plays a role in the regulation of cellular function and disease pathogenesis. The increasingly wealthy information on PTMs presents the challenge of systematically understanding the dynamics of PTM sites, with great opportunities to enlarge the target space by mechanisms underlying PTM allosteric regulation in drug design. Disclosed herein is a strategic framework and practical techniques involving integrating the sequence, structural topology, and particular dynamics features to characterize the functional context and druggabilities of PTM-associated pockets in proteins, which is exemplified with the well-known kinase target family.

The machine learning models with these biophysical features can be implemented to successfully classify the PTM residues and orthosteric residues On the other hand, PTMs were identified to be significantly enriched in the reported allosteric pockets and the allosteric potential of PTM pockets were thus proposed through these biophysical features. In the end, as an example of a successfully implementation, a covalent inhibitor DC-Srci-6668 targeting the PTM pocket in c-Src kinase was identified using virtual screening and in vitro assays. The crystal structure of c-Src with DC-Srci-6668 indicated this covalent inhibitor targeted the PTM pocket as predicted, inhibiting the phosphorylation and locking c-Src in the inactive state. The disclosed findings represent a valuable step toward PTM inspired drug design in kinase family, from highlighting the importance of dynamics of PTM residues on their allosteric potential, to identifying covalent inhibitor DC-Srci-6668 targeting the PTM pocket in c-Src as a successful application scenario.

Disclosed herein is a method for performing screening of pharmacophores or compounds for an allosteric interaction with a site of a protein, the method comprising: categorizing PTM features of a site of the protein into sequence features (SEQ), structural and topological features (SIR), and/or dynamic features (DYN); applying a machine learning model to analyze the SEQ, STR, and/or DYN features, the machine learning model trained to classify the site of the protein as an allosteric PTM pocket or a non-allosteric PTM pocket; and responsive to the classification of the site of the protein as an allosteric PTM pocket, applying a pharmacophore or a compound to the allosteric pocket via molecular modeling to determine a level of allosteric interaction between the pharmacophore or compound and the protein. In various embodiments, the sequence features (SEQ), structural and topological features (STR), and/or dynamic features (DYN) comprise each of sequence features (SEQ), structural and topological features (STR), and dynamic features (DYN), and wherein applying the machine learning model comprises applying the machine learning model to analyze the SEQ, STR and DYN features. In various embodiments, the molecular modeling comprises covalent docking of the pharmacophore or compound to the allosteric PTM pocket. In various embodiments, the molecular modeling comprises non-covalent docking of the pharmacophore or compound to the allosteric PTM pocket.

In various embodiments, categorizing PTM features comprises protein modeling. In various embodiments, the protein modeling comprises anisotropic network model (ANM) analysis. In various embodiments, the protein modeling comprises Gaussian network model (GNM) analysis. In various embodiments, the protein modeling comprises principal component analysis (PCA) analysis. In various embodiments, the machine learning model comprises a random forest (RF) model. In various embodiments, the machine learning model comprises a fully connected neural network (FCNN) model.

In various embodiments, the protein is an enzyme. In various embodiments, the enzyme is a kinase. In various embodiments, the kinase is of a family selected from the group consisting of: cyclin-dependent kinases (CDKs), Protein kinase B (AKTs), nonreceptor tyrosine kinases (NRTK), p21-activated kinases (PAKs), checkpoint kinases (CHKs), and receptor-interacting protein (RIP) kinases. In various embodiments, the PTM is of a type selected from the group consisting of: phosphorylation, glycosylation, ubiquitylation, acetylation, sumoylation, glutathionylation, methylation, succinylation, and S-nitrosylation. In various embodiments, the PTM is phosphorylation.

In various embodiments, the pharmacophore or compound is a de novo pharmacophore or compound. In various embodiments, the pharmacophore or compound is a known pharmacophore or compound. In various embodiments, all steps are performed in silico. In various embodiments, methods disclosed herein further comprise: performing a microscopic analysis, crystal structural analysis, and/or a biophysical assay to determine the level of allosteric interaction. In various embodiments, methods disclosed herein further comprise performing an in vitro and/or in vivo biological assay to confirm the level of allosteric interaction. In various embodiments, methods disclosed herein further comprise optimizing the de novo pharmacophore or compound to modify the interaction between the pharmacophore or compound with the protein, or to modify off-target effects of the pharmacophore or compound.

Additionally disclosed herein is a system or an apparatus comprising a non-transitory computer-readable memory, a processor and a communication interface wherein the processor is connected to the non-transitory computer-readable memory and the communication interface, wherein the processor is adapted to execute instructions stored on the non-transitory computer readable memory such that, when executed, cause the processor to perform or implement a method disclosed herein. Additionally disclosed herein is a pharmacophore or compound identified by a method disclosed herein.

Additionally disclosed herein is a method for classifying a post-translational modification (PTM) site on a protein, comprising: categorizing PTM features of the PTM site of the protein into sequence features (SEQ), structural and topological features (STR), and/or dynamic features (DYN); applying a machine learning model to analyze the SEQ, STR, and/or DYN features; and classifying the PTM site as an allosteric PTM pocket or non-allosteric PTM pocket. In various embodiments, wherein the sequence features (SEQ), structural and topological features (STR), and/or dynamic features (DYN) comprise each of sequence features (SEQ), structural and topological features (STR), and dynamic features (DYN), and wherein applying the machine learning model comprises applying the machine learning model to analyze the SEQ, STR and DYN features.

In various embodiments, categorizing PTM features comprises protein modeling. In various embodiments, the protein modeling comprises anisotropic network model (ANM) analysis. In various embodiments, the protein modeling comprises Gaussian network model (GNM) analysis. In various embodiments, the protein modeling comprises principal component analysis (PCA) analysis. In various embodiments, the machine learning model comprises a random forest (RF) model. In various embodiments, the machine learning model comprises a fully connected neural network (FCNN) model. In various embodiments, the protein is an enzyme. In various embodiments, the enzyme is a kinase. In various embodiments, the kinase is of a family selected from the group consisting of: cyclin-dependent kinases (CDKs), Protein kinase B (AKTs), nonreceptor tyrosine kinases (NRTK), p21-activated kinases (PAKs), checkpoint kinases (CHKs), and receptor-interacting protein (RIP) kinases. In various embodiments, the PTM is of a type selected from the group consisting of: phosphorylation, glycosylation, ubiquitylation, acetylation, sumoylation, glutathionylation, methylation, succinylation, and S-nitrosylation. In various embodiments, the PTM is phosphorylation.

In various embodiments, the machine learning model is trained to classify the site of the protein as one of an allosteric PTM pocket, an orthosteric residue, or other. In various embodiments, sequence features (SEQ) comprise one or more of residue identity features, conservation features, or co-evolution features. In various embodiments, structural and topological features (STR) comprise one or more of solvent accessibility features and features of node centralities calculated using weighted protein structure networks (PSNs). In various embodiments, the machine learning model more likely predicts that a site is an allosteric PTM pocket based on larger solvent accessibility feature values in comparison to smaller solvent accessibility feature values. In various embodiments, dynamic features (DYN) comprise one or more of b-factor features, square fluctuation features, cross-correlation features, and perturbation response scanning features. In various embodiments, the machine learning model more likely predicts that a site is an allosteric PTM pocket based on square fluctuation feature values in comparison to smaller square fluctuation feature values. In various embodiments, the machine learning model exhibits an area under the curve (AUC) value of at least 0.8. In various embodiments, the machine learning model exhibits an area under the curve (AUC) value of at least 0.9.

### BRIEF DESCRIPTION OF THE DRAWINGS

FIG. 1. Flowcharts illustrating exemplary analyses, simulation and modeling according to certain aspects of the invention.

FIG. 2. Various PTMs are involved in biological functions and diseases. (A) The percentage of main 7 PTM types in the collected kinase dataset. (B) The statistics of molecular functions for 6 PTM types. (C) The statistics of biological processes for 4 PTM types. (D) The percentage of different super-types of diseases in the kinase dataset. In total, 61 types of diseases are classified into 22 super-types based on the tissue information. (E) Distribution of PTM functions, biological processes and involved in diseases in kinases. In the phylogenetic tree of human kinome, the kinases are displayed in different sizes according to the number of PTMs involved in the molecular functions and biological processes. The large size corresponds to the great number of functional PTMs. According to the number of disease-related PTMs, the kinases are displayed as green circles (with one PTM or none), blue circles (with two PTMs), and red circles (with three PTMs). (F) The conceptive strategy in the research. The evolutionary, structural and dynamics features underlying the residue level and pocket level were characterized, emphasizing the potential allostery for PTM pockets in drug design.

FIG. 3. (A) PC1 modes (green arrows) and ANM1 modes (orange arrows) for CDK2 (PDB code: 4I3Z), AKT1 (PDB code: 4GVJ), c-Src (PDB code: 1Y16), PAK1 (PDB code: 4DAW), CHK2 (PDB code: 4BDK) and RIPK1 (PDB code: 6RLN), in which the red spheres represent the functional PTM sites and the blue spheres represent the PTM sites without functional report. (B) Comparison of the weighted sum of square displacements along PC1 and PC2 modes, with those predicted along all ANM modes, for the residues in kinases CDK2, AKT1, c-Src, PAK1, CHK2 and RIPK1.

FIG. 4. The comparative analysis of residue conservation, structural and dynamics features for PTM sites, orthosteric sites and others in kinase dataset. The violin plot for the (A) sequence conservation, (B) solvent-accessible surface area (ACC), (C) GNM square fluctuations (GNM-Sq), (D) Coevolution with orthosteric sites, (E) Cross-correlation with orthosteric sites, and (F) PRS sensors, comparing the PTM sites, orthosteric sites and other residues. P values were calculated by the Wilcoxon test. (G) Comparison of ROC plots generated for the three categories: PTMs, orthosteric sites and other residues, with SEQ, STR and DYN features alone and in combination respectively, by deep learning and random forest classifiers.

FIG. 5. (A) The crystal structure of CDK2 bound to allosteric inhibitor ANS (PDB code: 3PXF), and other allosteric compounds from recent research³¹. The PTM sites are represented as red spheres. (B) The crystal structure of ABL1-imatinib complex bound to compound 7 (ABL001 derivative) in the myristate binding pocket (PDB code: 6HD4), also with PTMs shown in red spheres (PDB code: 2FO0). (C) PTM residues are significantly enriched in reported allosteric pockets. (D-I) The comparative analysis of pocket features for PTM pockets, orthosteric pockets and non-PTM pockets in the kinase dataset. The violin plots for the druggability score (D), volume (E), conservation (F), coevolution with orthosteric sites (G), ANM_HF_bcc (H), and GNM_PRS_binding (I).

FIG. 6. (A) PTM pockets of c-Src kinase. The pocket features, including volume, conservation, ANM_HF_boc, and PRS_binding_col are represented by column figures. (B) The flow chart of covalent virtual screening targeting c-Src pocket 4. (C) The inhibition of compounds against c-Src catalytic activity at 50 μM evaluated by HTRF assay. (D) Compound DC-Srci-6668 exhibited an effective c-Src inhibitory activity with an IC₅₀ value of 2.387±0.164 μM by HTRF assay (n=3). (E) Compound DC-Srci-6668 inhibited the autophosphorylation of c-Src Y419 with an IC₅₀ value of 3.884±0.586 μM determined by ALPHA (n=3). (F) The melting curves of c-Src after DC-Srci-6668 treatment at the concentration ratios of 1:5, 1:10 and 1:20. (G) Intact protein mass of apo-c-Src (top panel) and DC-Srci-6668-treated c-Src (c-Src-DC-Srci-6668, bottom panel) determined by HPLC/MS analysis.

FIG. 7. (A) The complex crystal structure of c-Src and compound DC-Srci-6668 (gray, c-Src_DC-Srci-6668). The α-C helix and activation segment were superimposed with corresponding regions of inactive c-Src (cyan, PDB code: 2SRC) and active c-Src (pink, PDB code: 3DQW) (B) The 2Fo-Fc electron density map of compound DC-Srci-6668 with C280 residue. The contour level was set to 1.0 sigma. (C) Surface representation showed that compound DC-Srci-6668 fits well into the PTM pocket 4 of c-Src (green). (D) A close-up view showed the α-C helix of c-Src_DC-Srci-6668 adopts an “α-C out” orientation of inactive c-Src. (E) A close-up view showed the activation segment of c-Src_DC-Srci-6668 adopts a compact conformation of inactive c-Src. (F) A close-up view of the interactions between c-Src and compound DC-Srci-6668. The ligand and interacting residues are shown as sticks; hydrogen bonds are indicated by orange dotted lines. (G) 2D diagrams of interactions between c-Src and compound DC-Srci-6668. (H) The different binding mode of compound DC-Srci-6668 and ATP-competitive covalent kinase inhibitor SM1-71 (slate).

FIG. 8. RMSD distributions for the structural ensembles of the 6 kinases, including CKD2, AKT1, c-Src, PAK1, CHK2 and RIPK1 kinases. Figures show the RMSD distributions with respect to the respective reference structures for the datasets in Table 3. The RMSDs were based on the residues common to the reference structure and each superimposed structure. PTM represents the phosphorylated structures and Non-PTM represents the unphosphorylated structures.

FIG. 9. Projection of aligned structures onto PC1 and PC2 loadings, for 362 CDK2 structures, including 44 phosphorylated (red), and 318 unphosphorylated ones, for 25 AKT1 structures, including 10 phosphorylated (red) and 15 unphosphorylated ones, for 63 c-Src structures, including 7 phosphorylated (red) and 56 unphosphorylated ones, for 27 PAK1 structures, including 6 phosphorylated (red) and 21 unphosphorylated ones, for 34 CHK2 structures and for 14 RIPK1 structures.

FIG. 10. The violin plots for the features of PTM sites, orthosteric sites and other residues. The features described include ANM square fluctuations (ANM_Sq), the average coevolution values with orthosteric sites (SCA_bind_mean, MI_bind_mean, OMES_bind_mean), GNM all modes cross correlations with orthosteric sites (GNM_all_bec), ANM and GNM top3 modes (ANM top3 bec and GNM top3 bec), ANM and GNM low frequency modes (4-20 modes, ANM_LF_bcc and GNM_LF_bcc), ANM and GNM low-to-medium frequency modes (21-60 modes, ANM_LTIF_bcc and GNM_LTIF_bcc), ANM and GNM high modes frequency modes (>60 modes, ANM_HF_bcc and GNM_HF_bcc), ANM sensor, Shortest path, Betweenness, Closeness and Clustering coefficient.

FIG. 11. (A) Comparison of ROC plots generated for the three categories: PTM sites (labeled as class 1), orthosteric residues (labeled as class 2) and other residues, respectively, with the combinational features, by deep learning and random forest predictors. (B) Metrics for the performance of the models, which were calculated on the test set. (C) The confusion matrix for the three categories, by deep learning and random forest predictors.

FIG. 12. The violin plot for the features of PTM pockets, orthosteric pockets and other pockets. The features include pocket score, total solvent accessible surface area (Total_SASA), GNM high modes bcc (GNM_HF_bcc), GNM PRS mean values with orthosteric sites (GNM_PRS_binding_row), and Betweenness.

FIG. 13. (A) The structure of compound DC-Srci-6649. (B) Compound DC-Srci-6649 exhibited weak inhibition to c-Src catalytic activity with an IC₅₀ value of 24.78 μM. (C) The structure of compound DC-Srci-6905. (D) Compound DC-Srci-6905 exhibited weak inhibition to c-Src catalytic activity with an IC₅₀ value of 46.92 μM.

FIG. 14. Table 2.

FIG. 15. Table 6.

FIG. 16. A flowchart of the cDL framework for PAU and FuncPhos predictions.

FIG. 17. Performance of cDL, FNN and RF models in the prediction of PAU sites using independent test data sets. AUCs for phosphorylation site models (A), for acetylation site models (B), and for ubiquitination site models (C). (D) AUCs of various feature subsets for cDL models.

FIG. 18. Table 7. Prediction Metrics for PAU Sites in the Proposed Models, with the Ratio of Positive/Negative at 1:1 in Training Sets and Test Sets.

FIG. 19. Table 8. Prediction Metrics of Feature Subsets for PAU Sites in the Proposed Models, with the Ratio of Positive/Negative at 1:1 in Test Sets.

FIG. 20. Table 9. Prediction Metrics for the PAU Sites in Musite, PTMscape and DeepPhos Models, with the Ratio of Positive/Negative at 1:1 in Test Sets.

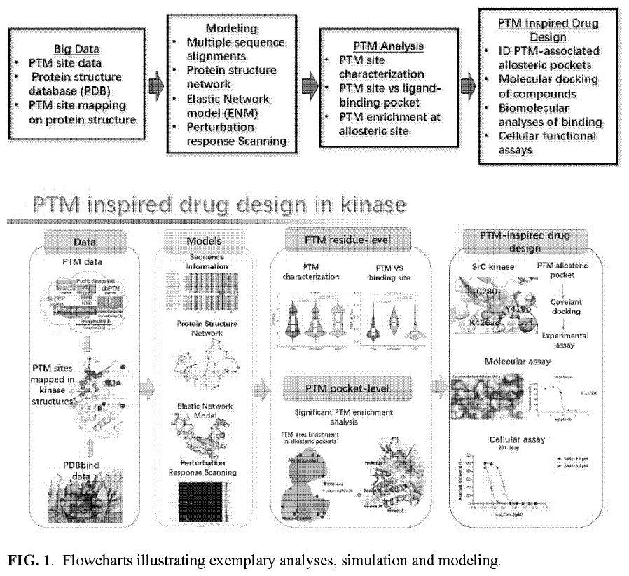

### DEFINITIONS

Unless defined otherwise, all technical and scientific terms used herein have the same meaning as commonly understood by one of ordinary skill in the art. Although any methods and materials similar or equivalent to those described herein can also be used in the practice or testing of the present disclosure, the preferred methods and materials are now described. Methods recited herein may be carried out in any order that is logically possible, in addition to a particular order disclosed.

As used herein, “at least” a specific value is understood to be that value and all values greater than that value.

The term “comprising”, when used to define compositions and methods, is intended to mean that the compositions and methods include the recited elements, but do not exclude other elements. The term “consisting essentially of”, when used to define compositions and methods, shall mean that the compositions and methods include the recited elements and exclude other elements of any essential significance to the compositions and methods. For example, “consisting essentially of” refers to administration of the pharmacologically active agents expressly recited and excludes pharmacologically active agents not expressly recited. The term consisting essentially of does not exclude pharmacologically inactive or inert agents, e.g., pharmaceutically acceptable excipients, carriers or diluents. The term “consisting of”, when used to define compositions and methods, shall mean excluding trace elements of other ingredients and substantial method steps. Embodiments defined by each of these transition terms are within the scope of this invention.

In this specification and the appended claims, the singular forms “a,” “an,” and “the” include plural reference, unless the context clearly dictates otherwise.

As used herein, the term “computer” refers to any device with processing capability such that it can execute instructions. Those skilled in the art will realize that such processing capabilities are incorporated into many different devices.

Where a computing device is illustrated as a local device, it should be appreciated that the computing device may be located remotely and accessed via a network or other communication link or interface. Alternatively, a local computer may download pieces of the software as needed, or execute some software instructions at the local terminal and some at the remote computer or computer network.

As used herein, the terms “administration” of or “administering” a disclosed compound encompasses the delivery to a subject of a compound as described herein, or a prodrug or other pharmaceutically acceptable form thereof, using any suitable formulation or route of administration, as discussed herein.

As used herein, the term “alkyl” refers to a straight or branched hydrocarbon chain radical consisting solely of carbon and hydrogen atoms, containing no unsaturation, having from one to ten carbon atoms (e.g., C₁₋₁₀ alkyl). Whenever it appears herein, a numerical range such as “1 to 10” refers to each integer in the given range; e.g., “1 to 10 carbon atoms” means that the alkyl group can consist of 1 carbon atom, 2 carbon atoms, 3 carbon atoms, etc., up to and including 10 carbon atoms, although the present definition also covers the occurrence of the term “alkyl” where no numerical range is designated. In some embodiments, “alkyl” can be a C₁₆ alkyl group. In some embodiments, alkyl groups have 1 to 10, 1 to 8, 1 to 6, or 1 to 3 carbon atoms. Representative saturated straight chain alkyls include, but are not limited to, -methyl, -ethyl, -n-propyl, -n-butyl, -n-pentyl, and -n-hexyl; while saturated branched alkyls include, but are not limited to, -isopropyl, -sec-butyl, -isobutyl, -tert-butyl, -isopentyl, 2-methylbutyl, 3-methylbutyl, 2-methylpentyl, 3-methylpentyl, 4-methylpentyl, 2-methylhexyl, 3-methylhexyl, 4-methylhexyl, 5-methylhexyl, 2,3-dimethylbutyl, and the like.

As used herein, “alkylene” refers to a divalent radical of an alkyl group.

The term “aryl” is art-recognized and refers to a carbocyclic or heterocyclic aromatic group. In some embodiments, an aryl may be phenyl or 5-6 membered heteroaryl (e.g., thiophenyl).

The term “aliphatic” or “aliphatic group,” as used herein, means a straight-chain (i.e., unbranched) or branched, substituted or unsubstituted hydrocarbon chain that is completely saturated or that contains one or more units of unsaturation, or a monocyclic hydrocarbon or bicyclic hydrocarbon that is completely saturated or that contains one or more units of unsaturation, but which is not aromatic, that has a single point of attachment to the rest of the molecule. In some embodiments, aliphatic groups contain 3-8 aliphatic carbon atoms.

The terms “disease,” “disorder” and “condition” are used interchangeably unless indicated otherwise.

As used herein, the term “halogen” or “halo” refers to fluorine (F), chlorine (Cl), bromine (Br) and iodine (I).

As used herein, the term “therapeutically effective amount” refer to that amount of a compound or pharmaceutical composition described herein that is sufficient to effect the intended application including, but not limited to, disease treatment, as illustrated below.

In some embodiments, the amount is that effective for stop the progression or effect reduction of an inflammatory disease or disorder. In some embodiments, the amount is that effective for stop the progression or effect reduction of an immune system disorders. In some embodiments, the amount is that effective to stop the progression or effect reduction of an autoimmune disease or disorder. In some embodiments, the amount is that effective for stop the progression or effect reduction of a cardiovascular disease or disorder. In some embodiments, the amount is that effective for detectable killing or inhibition of the growth or spread of cancer cells; the size or number of tumors; or other measure of the level, stage, progression or severity of the cancer. In some embodiments, the amount is that effective for stop the progression or effect reduction of PPD, depression, insomnia, sleep apnea, restless legs syndrome, and narcolepsy, emotional disorders, depression, schizophrenia, bipolar disorder, obsessive-compulsive disorder, and other anxiety disorders, behavioral and pharmacological syndrome of dementia, or neurodegenerative diseases. In some embodiments, the amount is that effective for stop the progression or effect reduction of Parkinson's disease (PD). In some embodiments, the amount is that effective for stop the progression or effect reduction of Alzheimer's disease (AD).

The therapeutically effective amount can vary depending upon the intended application, or the subject and disease condition being treated, e.g., the desired biological endpoint, the pharmacokinetics of the compound, the disease being treated, the mode of administration, and the weight and age of the patient, which can readily be determined by one of ordinary skill in the art. The term also applies to a dose that will induce a particular response in target cells, e.g., reduction of cell migration. The specific dose will vary depending on, for example, the particular compounds chosen, the species of subject and their age/existing health conditions or risk for health conditions, the dosing regimen to be followed, the severity of the disease, whether it is administered in combination with other agents, timing of administration, the tissue to which it is administered, and the physical delivery system in which it is carried.

The term “optionally substituted” is understood to mean that a given chemical moiety (e.g. an alkyl group) can (but is not required to) be bonded other substituents (e.g. heteroatoms). For instance, an alkyl group that is optionally substituted can be a fully saturated alkyl chain (i.e. a pure hydrocarbon). Alternatively, the same optionally substituted alkyl group can have substituents different from hydrogen. For instance, it can, at any point along the chain be bounded to a halogen atom, a hydroxyl group, or any other substituent described herein. Thus, the term “optionally substituted” means that a given chemical moiety has the potential to contain other functional groups, but does not necessarily have any further functional groups. Suitable substituents used in the optional substitution of the described groups include, without limitation, halogen, oxo, CN, —COOH, —CH₂CN, —O—C₁-C₆ alkyl, C₁-C₆ alkyl, —OC₁-C₆ alkenyl, —OC₁-C₆ alkynyl, —C₁-C₆ alkenyl, —C₁-C₆ alkynyl, —OH, —OP(O)(OH)₂, —OC(O)C₁-C₆ alkyl, —C(O)C₁-C₆ alkyl, —OC(O)OC₁-C₆ alkyl, NH₂, NH(C₁-C₆ alkyl), N(C₁-C₆ alkyl)₂, —NHC(O)C₁-C₆ alkyl, —C(O)NHC₁-C₆ alkyl, —S(O)₂—C₁-C₆ alkyl, —S(O)NHC₁-C₆ alkyl, and S(O)N(C₁-C₆ alkyl)₂.

As used herein, a “pharmaceutically acceptable form” of a disclosed compound includes, but is not limited to, pharmaceutically acceptable salts, esters, hydrates, solvates, isomers, prodrugs, and isotopically labeled derivatives of disclosed compounds. In one embodiment, a “pharmaceutically acceptable form” includes, but is not limited to, pharmaceutically acceptable salts, esters, isomers, prodrugs and isotopically labeled derivatives of disclosed compounds. In some embodiments, a “pharmaceutically acceptable form” includes, but is not limited to, pharmaceutically acceptable salts, esters, stereoisomers, prodrugs and isotopically labeled derivatives of disclosed compounds.

In certain embodiments, the pharmaceutically acceptable form is a pharmaceutically acceptable salt. As used herein, the term “pharmaceutically acceptable salt” refers to those salts which are, within the scope of sound medical judgment, suitable for use in contact with the tissues of subjects without undue toxicity, irritation, allergic response and the like, and are commensurate with a reasonable benefit/risk ratio. Pharmaceutically acceptable salts are well known in the art. For example, Berge et al. describes pharmaceutically acceptable salts in detail in J. Pharmaceutical Sciences (1977) 66:1-19. Pharmaceutically acceptable salts of the compounds provided herein include those derived from suitable inorganic and organic acids and bases. Examples of pharmaceutically acceptable, nontoxic acid addition salts are salts of an amino group formed with inorganic acids such as hydrochloric acid, hydrobromic acid, phosphoric acid, sulfuric acid and perchloric acid or with organic acids such as acetic acid, oxalic acid, maleic acid, tartaric acid, citric acid, succinic acid or malonic acid or by using other methods used in the art such as ion exchange. Other pharmaceutically acceptable salts include adipate, alginate, ascorbate, aspartate, benzenesulfonate, besylate, benzoate, bisulfate, borate, butyrate, camphorate, camphorsulfonate, citrate, cyclopentanepropionate, digluconate, dodecylsulfate, ethanesulfonate, formate, fumarate, glucoheptonate, glycerophosphate, gluconate, hemisulfate, heptanoate, hexanoate, hydroiodide, 2-hydroxy-ethanesulfonate, lactobionate, lactate, laurate, lauryl sulfate, malate, maleate, malonate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, p-toluenesulfonate, undecanoate, valerate salts, and the like. In some embodiments, organic acids from which salts can be derived include, for example, acetic acid, propionic acid, glycolic acid, pyruvic acid, oxalic acid, lactic acid, trifluoracetic acid, maleic acid, malonic acid, succinic acid, fumaric acid, tartaric acid, citric acid, benzoic acid, cinnamic acid, mandelic acid, methanesulfonic acid, ethanesulfonic acid, p-toluenesulfonic acid, salicylic acid, and the like.

The salts can be prepared in situ during the isolation and purification of the disclosed compounds, or separately, such as by reacting the free base or free acid of a parent compound with a suitable base or acid, respectively. Pharmaceutically acceptable salts derived from appropriate bases include alkali metal, alkaline earth metal, ammonium and N⁺(C₁₋₄alkyl)⁴ salts. Representative alkali or alkaline earth metal salts include sodium, lithium, potassium, calcium, magnesium, iron, zinc, copper, manganese, aluminum, and the like. Further pharmaceutically acceptable salts include, when appropriate, nontoxic ammonium, quaternary ammonium, and amine cations formed using counterions such as halide, hydroxide, carboxylate, sulfate, phosphate, nitrate, lower alkyl sulfonate and aryl sulfonate. Organic bases from which salts can be derived include, for example, primary, secondary, and tertiary amines, substituted amines, including naturally occurring substituted amines, cyclic amines, basic ion exchange resins, and the like, such as isopropylamine, trimethylamine, diethylamine, triethylamine, tripropylamine, and ethanolamine. In some embodiments, the pharmaceutically acceptable base addition salt can be chosen from ammonium, potassium, sodium, calcium, and magnesium salts.

In certain embodiments, the pharmaceutically acceptable form is a pharmaceutically acceptable ester. As used herein, the term “pharmaceutically acceptable ester” refers to esters that hydrolyze in vivo and include those that break down readily in the human body to leave the parent compound or a salt thereof. Such esters can act as a prodrug as defined herein. Pharmaceutically acceptable esters include, but are not limited to, alkyl, alkenyl, alkynyl, aryl, aralkyl, and cycloalkyl esters of acidic groups, including, but not limited to, carboxylic acids, phosphoric acids, phosphinic acids, sulfinic acids, sulfonic acids and boronic acids. Examples of esters include formates, acetates, propionates, butyrates, acrylates and ethylsuccinates. The esters can be formed with a hydroxy or carboxylic acid group of the parent compound.

In certain embodiments, the pharmaceutically acceptable form is a “solvate” (e.g., a hydrate). As used herein, the term “solvate” refers to compounds that further include a stoichiometric or non-stoichiometric amount of solvent bound by non-covalent intermolecular forces. The solvate can be of a disclosed compound or a pharmaceutically acceptable salt thereof. Where the solvent is water, the solvate is a “hydrate”. Pharmaceutically acceptable solvates and hydrates are complexes that, for example, can include 1 to about 100, or 1 to about 10, or 1 to about 2, about 3 or about 4, solvent or water molecules. It will be understood that the term “compound” as used herein encompasses the compound and solvates of the compound, as well as mixtures thereof.

In certain embodiments, the pharmaceutically acceptable form is a prodrug. As used herein, the term “prodrug” (or “pro-drug”) refers to compounds that are transformed in vivo to yield a disclosed compound or a pharmaceutically acceptable form of the compound. A prodrug can be inactive when administered to a subject, but is converted in vivo to an active compound, for example, by hydrolysis (e.g., hydrolysis in blood). In certain cases, a prodrug has improved physical and/or delivery properties over the parent compound. Prodrugs can increase the bioavailability of the compound when administered to a subject (e.g., by permitting enhanced absorption into the blood following oral administration) or which enhance delivery to a biological compartment of interest (eg., the brain or lymphatic system) relative to the parent compound. Exemplary prodrugs include derivatives of a disclosed compound with enhanced aqueous solubility or active transport through the gut membrane, relative to the parent compound.

The prodrug compound often offers advantages of solubility, tissue compatibility or delayed release in a mammalian organism (see, e.g., Bundgard, H., Design of Prodrugs (1985), pp. 7-9, 21-24 (Elsevier, Amsterdam). A discussion of prodrugs is provided in Higuchi, T., et al., “Pro-drugs as Novel Delivery Systems,” A.C.S. Symposium Series, Vol. 14, and in Bioreversible Carriers in Drug Design, ed. Edward B. Roche, American Pharmaceutical Association and Pergamon Press, 1987, both of which are incorporated in full by reference herein. Exemplary advantages of a prodrug can include, but are not limited to, its physical properties, such as enhanced water solubility for parenteral administration at physiological pH compared to the parent compound, or it can enhance absorption from the digestive tract, or it can enhance drug stability for long-term storage.

As used herein, the term “pharmaceutically acceptable” excipient, carrier, or diluent refers to a pharmaceutically acceptable material, composition or vehicle, such as a liquid or solid filler, diluent, excipient, solvent or encapsulating material, involved in carrying or transporting the subject pharmaceutical agent from one organ, or portion of the body, to another organ, or portion of the body. Each carrier must be “acceptable” in the sense of being compatible with the other ingredients of the formulation and not injurious to the patient. Some examples of materials which can serve as pharmaceutically-acceptable carriers include: sugars, such as lactose, glucose and sucrose; starches, such as corn starch and potato starch; cellulose, and its derivatives, such as sodium carboxymethyl cellulose, ethyl cellulose and cellulose acetate; powdered tragacanth; malt; gelatin; talc; excipients, such as cocoa butter and suppository waxes; oils, such as peanut oil, cottonseed oil, safflower oil, sesame oil, olive oil, corn oil and soybean oil; glycols, such as propylene glycol; polyols, such as glycerin, sorbitol, mannitol and polyethylene glycol; esters, such as ethyl oleate and ethyl laurate; agar; buffering agents, such as magnesium hydroxide and aluminum hydroxide; alginic acid; pyrogen-free water; isotonic saline; Ringer's solution; ethyl alcohol; phosphate buffer solutions; and other non-toxic compatible substances employed in pharmaceutical formulations. Wetting agents, emulsifiers and lubricants, such as sodium lauryl sulfate, magnesium stearate, and polyethylene oxide-polypropylene oxide copolymer as well as coloring agents, release agents, coating agents, sweetening, flavoring and perfuming agents, preservatives and antioxidants can also be present in the compositions.

As used herein, the term “subject” refers to any animal (e.g., a mammal), including, but not limited to humans, non-human primates, rodents, and the like, which is to be the recipient of a particular treatment. Typically, the terms “subject” and “patient” are used interchangeably herein in reference to a human subject.

As used herein, the terms “treatment” or “treating” a disease or disorder refers to a method of reducing, delaying or ameliorating such a condition before or after it has occurred. Treatment may be directed at one or more effects or symptoms of a disease and/or the underlying pathology. Treatment is aimed to obtain beneficial or desired results including, but not limited to, therapeutic benefit and/or a prophylactic benefit. By therapeutic benefit is meant eradication or amelioration of the underlying disorder being treated. Also, a therapeutic benefit is achieved with the eradication or amelioration of one or more of the physiological symptoms associated with the underlying disorder such that an improvement is observed in the patient, notwithstanding that the patient can still be afflicted with the underlying disorder. For prophylactic benefit, the pharmaceutical compounds and/or compositions can be administered to a patient at risk of developing a particular disease, or to a patient reporting one or more of the physiological symptoms of a disease, even though a diagnosis of this disease may not have been made. The treatment can be any reduction and can be, but is not limited to, the complete ablation of the disease or the symptoms of the disease. As compared with an equivalent untreated control, such reduction or degree of prevention is at least 5%, 10%, 20%, 40%, 50%, 60%, 80%, 90%, 95%, or 100% as measured by any standard technique.

### DETAILED DESCRIPTION OF THE INVENTION

The invention is based in part on a novel approach to in silico-based drug discovery. In particular, the disclosed systems and methods integrate PTM sequence, structural topology and dynamics features with protein modeling and machining learning techniques to afford efficient and accurate characterization and classification of PTM sites. This PTM inspired drug design and screening approach offers unique capabilities for identification of useful pharmacophores and drug candidates.

A key feature of the PTM inspired drug design and screening herein disclosed is to take into consideration of the functional diversity of PTM isoforms and the dynamics induced by PTMs. Taking kinases for example, disease often occurs through PTMs or mutations that shift the kinase population from an OFF to a functional ON state, with the ramifications propagating through the cellular pathways to affect the cell state¹³. Kinases thus play a central role in a large number of physiological processes and have been implicated in the pathogenesis of many diseases, becoming the attractive targets in both academia and pharmaceutical industry.

Kinases share a highly conserved catalytic core that folds into a similar bi-lobar three-dimensional structure. The drug selectivity and quickly acquired resistance have been core problems in kinase drug design for many years. Various PTMs on kinases have been shown to be involved in molecular functions, cellular processes, and have been highly correlated with diseases. For example, in c-Src, the phosphorylations at Y419 and Y530 are essential in regulating its activation process. The SH2 domain binds to the phosphorylated Y530 at the C-terminal, forming a clamp with the SH3 domain and resulting in an inactive state¹⁵. The dephosphorylation of Y530 allows the dissociation and subsequent phosphorylation at Y419, and initiates a conformational reorganization of the activation loop, contributing to the switch from the inactive to a fully active state. Once activated, c-Src can regulate multiple downstream signaling pathways, such as RAS/MAPK, PI3K/AKT and STAT pathways¹⁷. The dysregulation of c-Src is therefore considered as an oncogenic signature and a driving force for cancer initiation, including colon, triple-negative breast, non-small cell lung, and head and neck cancers^{14,18}.

However, small-molecule inhibitors targeting the ATP-binding pocket frequently encounter poor therapeutic effects due to the emergence of drug resistance mutations. Hence, the introduction of PTMs in kinases would enlarge the conserved biological structural space for drug design.

Although several simulations have been made on the conformational fluctuations of PTMs in specific proteins, the systematic characterization of protein dynamics underlying PTM sites is still poor for PTM functional research, which extremely limits the applications for PTM-related diseases and PTM-inspired drug design. In the systematic characterization of protein dynamics, sequence information and network models are increasingly used as the bridge for molecular research and systems biology.

The present inventors systematically elaborated the theories, tools and applications of network models in the high-throughput modeling of protein dynamics and allosteric regulation in a recent research⁹. Amongst, elastic network models (ENMs) and protein structure networks (PSNs) are representative methods for capturing protein dynamics and quantitative structural topologies in protein allosteric regulation²⁰, protein-protein interaction (PPI) hotspot and missense mutant identification²¹, as well as the allosteric pocket discovery²². Collectively, deciphering information on protein dynamics with structural and evolutionary features can lead to an improved understanding of the allosteric regulation involving PTMs, which has the potential for PTM-inspired drug design.

Herein, a novel strategy based on the proposed “dynamics-allostery-drug design” paradigm for PTM research is disclosed. The evolutionary, structural and dynamics features were characterized for these PTM sites, and emphasized the potential allostery for PTM pockets in drug design.

The results indicated that PTM sites, mainly phosphorylation sites, possessed a certain degree of conservation, as well as high allosteric potential for kinase regulation The machine learning models supported the characterization of PTM residues, with dynamics and allosteric features. To support the strategy of PTM inspired drug design in kinase family, c-Src kinase was used as a case study to target the PTM pocket 4, with high allosteric potential. Through covalent docking based virtual screening and biochemical assays, a covalent inhibitor targeting the PTM pocket was identified. The crystal structure of c-Src with the covalent inhibitor supported the predicted binding mode and inhibitory mechanism. The research systematically complemented the biophysics principle underlying PTMs in kinase family, enriched understanding of PTM functions, and supported the strategy of PTM inspired drug design.

In one aspect, the invention generally relates to a method for evaluating a pharmacophore or a compound for allosteric interaction with a post-translational modification (PTM) pocket on a protein. The method comprises: categorizing PTM features into sequence features (SEQ), structural and topological features (STR), and/or dynamic features (DYN); applying machine learning modeling on SEQ, STR and/or DYN features; classifying residues as allosteric PTM pockets, orthosteric residues, or others; and applying a pharmacophore or a compound to an allosteric PTM pocket via molecular modeling to determine a level of allosteric interaction between the pharmacophore or compound and the protein.

In certain embodiments, the method comprises: categorizing PTM features into SEQ, STR and DYN features; and applying machine learning modeling on SEQ, STR and DYN features.

Machine learning refers to algorithms that give a computer the ability to learn without being explicitly programmed including algorithms that can learn from and make predictions about data. Machine learning thus is the ability of a learning machine to perform accurately on new, unseen examples/tasks after having experienced a learning data set. Exemplary machine learning algorithms include, but not limited to, decision tree learning, artificial neural networks, deep learning neural network, support vector machines, rule base machine learning, random forest, nearest neighbor, support vector classifier, partial least square, and logistic regression. Examples of neural networks include, but not limited to, convolutional neural networks, deep convolutional neural networks, cascaded deep convolutional neural networks, graph convolutional neural networks (GCNN), etc.

In certain embodiments, molecular modeling comprises covalent docking of the pharmacophore or compound to the allosteric PTM site.

In certain embodiments, molecular modeling comprises non-covalent docking of the pharmacophore or compound to the allosteric PTM site.

In certain embodiments, categorizing PTM features comprises protein modeling.

In certain embodiments, protein modeling comprises anisotropic network model (ANM) analysis, Gaussian network model (GNM) analysis, and/or principal component analysis (PCA) analysis.

ANM refers to an elastic network model (coarse-grained normal mode analysis) for proteins and other biomolecules with resolution at the level of residues. This model computes the principle modes of motion and likely conformational change directions for such molecules. (See, e.g., Atilgan et al., 2001, “Anisotropy of fluctuation dynamics of proteins with an elastic network model,” Biophys J 80 (1):505-15; Doruker, et al. 2000, “Dynamics of proteins predicted by molecular dynamics simulations and analytical approaches: application to alpha-amylase inhibitor,” Proteins, 15, 512-524.)

GNM is a representation of a biological macromolecule as an elastic mass-and-spring network to study, understand, and characterize the mechanical aspects of its long-time large-scale dynamics. (See, e.g., Bahar, et al. 1997, “Direct evaluation of thermal fluctuations in proteins using a single-parameter harmonic potential,” Fold Des, 2, 173-181; Haliloglu, et al. 1997 “Gaussian dynamics of folded proteins,” Phys. Rev. Lett. 79 (16): 3090-3093.)

PCA refers to a mathematical procedure that uses an orthogonal transformation to convert a set of observations of possibly correlated variables into a set of values of linearly uncorrelated variables called principal components. The number of principal components is less than or equal to the number of original variables. This transformation is defined in such a way that the first principal component has the largest possible variance (i.e., accounts for as much of the variability in the data as possible), and each succeeding component in turn has the highest variance possible under the constraint that it be orthogonal to (i.e., uncorrelated with) the preceding components.

In certain embodiments, machine learning model comprises a random forest (RF) model.

RF refers to a combination of classification tree predictors such that each tree depends on the values of a random vector sampled independently and with the same distribution for all trees in the forest. A random forest is a learning ensemble consisting of a bagging of un-pruned decision tree learners with a randomized selection of features at each split of the decision tree. A random forest grows a large number of classification trees, each of which votes for the most popular class. The random forest then classifies a variable by taking the most popular voted class from all the tree predictors in the forest.

In certain embodiments, the deep learning model utilizes a fully connected neural network (FCNN) model.

In certain embodiments, the protein is an enzyme. In certain embodiments, the enzyme is a kinase. In certain embodiments, the kinase is of a family selected from the group consisting of: cyclin-dependent kinases (CDKs), Protein kinase B (AKTs), non-receptor tyrosine kinases (NRTK), p21-activated kinases (PAKs), checkpoint kinases (CHKs), and receptor-interacting protein (RIP) kinases.

A variety of PTMs may be analyzed using the disclosed methods.

Various types of PTMs are known in the art. In certain embodiments, the PTM is of a type selected from the group consisting of: phosphorylation, glycosylation, ubiquitylation, acetylation, sumoylation, glutathionylation, methylation, succinylation, and S-nitrosylation.

Reversible protein phosphorylation, principally on serine, threonine or tyrosine residues, is among the most important and well-studied PTMs. Phosphorylation plays key roles in the regulation of many cellular processes, including cell cycle, growth, apoptosis and signal transduction pathways.

In certain embodiments, the relevant PTM in a disclosed method is phosphorylation.

Protein glycosylation plays significant roles in protein folding, conformation, distribution, stability and activity. Glycosylation encompasses a diverse selection of sugar-moiety additions to proteins that ranges from simple monosaccharide modifications of nuclear transcription factors to highly complex branched polysaccharide changes of cell surface receptors. Carbohydrates in the form of aspargine-linked (N-linked) or serine/threonine-linked (O-linked) oligosaccharides are major structural components of many cell surface and secreted proteins.

In certain embodiments, the relevant PTM in a disclosed method is glycosylation.

Ubiquitin is a small (8.6 kDa) regulatory protein found in most tissues of eukaryotic organisms. Ubiquitylation, the addition of ubiquitin to a substrate protein, affects proteins in many ways: it can mark them for degradation via the proteasome, alter their cellular location, affect their activity, and promote or prevent protein interactions. Ubiquitylation involves three main steps: activation, conjugation, and ligation, performed by ubiquitin-activating enzymes (E1s), ubiquitin-conjugating enzymes (E2s), and ubiquitin ligases (E3s), respectively. The result of this sequential cascade is to bind ubiquitin to lysine residues on the protein substrate via an isopeptide bond, cysteine residues through a thioester bond, serine and threonine residues through an ester bond, or the amino group of the protein's N-terminus via a peptide bond.

In certain embodiments, the relevant PTM in a disclosed method is ubiquitylation.

The regulation of transcription factors, effector proteins, molecular chaperones, and cytoskeletal proteins by acetylation and deacetylation is a significant post-translational regulatory mechanism. N-terminal acetylation is among the most common co-translational covalent modifications of proteins and plays a role in the synthesis, stability and localization of proteins. About 85% of all human proteins are acetylated at their Na-terminus.

Proteins are typically acetylated on lysine residues and this reaction relies on acetyl-coenzyme A as the acetyl group donor. In histone acetylation and deacetylation, histone proteins are acetylated and deacetylated on lysine residues in the N-terminal tail as part of gene regulation.

In certain embodiments, the relevant PTM in a disclosed method is acetylation.

Methylation refers to the transfer of one-carbon methyl groups to nitrogen or oxygen (N- and O-methylation, respectively) to amino acid side chains to increase the hydrophobicity of the protein and neutralize a negative amino acid charge when bound to carboxylic acids. Amino acid residues can be conjugated to a single methyl group or multiple methyl groups to increase the effects of modification. Methylation is mediated by methyltransferases, and S-adenosyl methionine (SAM) is the primary methyl group donor.

In certain embodiments, the relevant PTM in a disclosed method is methylation.

Sumoylation is involved in various cellular processes, such as nuclear-cytosolic transport, transcriptional regulation, apoptosis, protein stability, response to stress, and progression through the cell cycle. SUMO proteins are similar to ubiquitin and are considered members of the ubiquitin-like protein family. Sumoylation is directed by an enzymatic cascade analogous to that involved in ubiquitination. In contrast to ubiquitin, SUMO is not used to tag proteins for degradation. Mature SUMO is produced when the last four amino acids of the C-terminus have been cleaved off to allow formation of an isopeptide bond between the C-terminal glycine residue of SUMO and an acceptor lysine on the target protein.

In certain embodiments, the relevant PTM in a disclosed method is sumoylation.

S-glutathionylation refers to a post-translational modification forming mixed disulfides between protein reactive thiols and glutathione. S-glutathionylation regulates redox-based signaling events in the cell and serves as a protective mechanism against oxidative damage. S-glutathionylation alters protein function, interactions, and localization across physiological processes, and its aberrant function is implicated in various human diseases.

In certain embodiments, the relevant PTM in a disclosed method is glutathionylation.

Succinylation refers to a posttranslational modification where a succinyl group (—CO—CH₂—CH₂—CO₂H) is added to a lysine residue of a protein molecule. This modification is found in many proteins, including histones.

In certain embodiments, the relevant PTM in a disclosed method is succinylation.

S-nitrosylation is a fundamental mechanism for cellular signaling across phylogeny and accounts for the large part of NO bioactivity. It involves the covalent attachment of a nitric oxide group (—NO) to cysteine thiol within a protein to form an S-nitrosothiol (SNO). S-nitrosylation has diverse regulatory roles in bacteria, yeast and plants and in all mammalian cells.

In certain embodiments, the relevant PTM in a disclosed method is S-nitrosylation.

The present invention allows identification of compounds that can interact with a protein or enzyme at one or more of its PTM allosteric sites. The molecular modelling and drug design techniques may involve de novo compound design. In certain embodiments, the de novo compound design involves the identification of functional groups, molecular fragments and/or pharmacophores which can interact with PTM allosteric sites. In certain embodiments, the de novo compound design involves linking functional groups, molecular fragments and/or pharmacophores to form a single compound.

In certain embodiments, the pharmacophore or compound is a de novo pharmacophore or compound.

In certain embodiments, the pharmacophore or compound is a known pharmacophore or compound.

The identified compounds, with or without further modification or optimization, may be useful as a pharmaceutical agent. Compounds so identified may be useful in the manufacture of a medicament for treating a disease or condition associated with the respective protein or enzyme. Thus, the invention encompasses such compounds and pharmaceutical compositions and methods of treatment thereof.

With the exception of certain biophysical or biological assays or testing that require physical samples and experimentation, all aspects of the disclosed method can be performed in silico (i.e., experimentation and/or analysis performed by computer) including certain in silico biophysical or biological assays.

The present invention includes confirming or validating in silico binding of a chemical compound via microscopic analysis, crystal structural analysis, and/or a biophysical assay. In certain embodiments, the disclosed method further comprises: performing a microscopic analysis, crystal structural analysis, and/or a biophysical assay to determine the level of allosteric interaction.

The present invention also includes determining the efficacy of a chemical compound identified in an in vitro biological assay or in vivo in a subject. In certain embodiments, the disclosed method further comprises: performing an in vitro and/or in vivo biological assay to confirm the level of allosteric interaction.

The disclosed method may further includes determining if a chemical compound has or presents a risk of toxicity, off-target effect or any adverse drug reaction via in silico, in vitro or in vivo assays. In silico methods for determining off-target effects are known in the art. In vitro methods for determining off-target effects are also known in the art.

In certain embodiments, the disclosed method further comprises: optimizing the de novo pharmacophore or compound to modify the interaction between the pharmacophore or compound with the protein, or to modify off-target effects of the pharmacophore or compound.

Molecular modelling techniques useful for may employ automated docking algorithms.

Software packages useful for implementing molecular modelling techniques include: Multiple sequence alignments (MSA) by Clustal Omega. Shannon entropy for each position in the MSA to assess the conservation of residues was calculated using Evol, a python module in ProDy package. DSSP software was used to calculate solvent accessibility and to assign the secondary structures. Fpocket software was used to predict cavities or pockets and to identify residues that were located in pockets. Bio3D (R package) was used to model the protein structure networks (PSNs). The elastic network model (ENM) was produced with Anisotropic Network Model (ANM) and Gaussian Network Model (GNM) from the ProDy package. adapted to elucidate the equilibrium dynamics of protein structures.

Modelling may include one or more steps of energy minimization with standard molecular mechanics force fields, such as Tripos force field parameters. Docking was performed using covalent docking module of the Schrodinger software package. Electrostatic and Van der Waals energy were the main provisions of the scoring functions. For the calculation of electrostatic energy, the atomic charges for the protein were calculated by the Protein Preparation Wizard module from Schrodinger package with Tripos force field parameters. For the calculation of Van der Waals energy, the Lennard-Jones (6-12) potential was used.

In silico compounds libraries may be screened for their ability to interact with a PTM allosteric pocket by using their respective atomic co-ordinates in automated docking algorithms.

Various types of algorithms for detecting, measuring and/or analyzing binding pockets on proteins exists in the art, for example, geometric algorithms, energy-based methods, and precedence-based methods, including Fpocket software and the methods described herein.

Various docketing algorithms are known in the art. Exemplary docking algorithms include Affinity, Autodock, Combibuild, Dockvision, Fred, Flexidock, Flex-X, Glide, Gold.

In another aspect, the invention generally relates to a pharmacophore or compound identified by a method disclosed herein.

In yet another aspect, the invention generally relates to a method for characterizing post-translational modification (PTM) sites on a protein using PTM features including sequence features (SEQ), structural and topological features (STR), and/or dynamic features (DYN) of PTM sites.

In yet another aspect, the invention generally relates to a system or an apparatus comprising a non-transitory computer-readable memory, a processor and a communication interface wherein the processor is connected to the non-transitory computer-readable memory and the communication interface, wherein the processor is adapted to execute instructions stored on the non-transitory computer-readable memory such that, when executed, cause the processor to perform or implement a method disclosed herein.

A system or apparatus of the invention can be constructed such that it is a stand-alone computer for access by a user. Alternatively, the system or apparatus can be implemented on different types of processing devices. Software instructions can include source code, object code, machine code, or any other stored data that is operable to cause a processing system to execute a method disclosed herein.

Software instructions and data can be stored in different types of computer-implemented storage devices and programming constructs (e.g., RAM, ROM, flash memory, databases, etc.). Systems and methods of the invention can be provided on different types of computer-readable media such as, CD-ROM, diskette, RAM, flash memory, computer's hard drive, etc.).

It is noted that, although a system or apparatus is illustrated as a single system, it is to be understood that the computing device can be a distributed system. Several devices, for example, can be configured such that they are in communication by way of a network connection and can cooperatively perform tasks described as being performed or executed by a computing device.

Those skilled in the art will realize that storage devices utilized to store program instructions can be distributed remotely and/or across a network. For example, a remote computer may store an example of the process described as software. A local or terminal computer may access the remote computer and download a part or all of the software to run the program.

### Compounds

In another aspect, provided herein is a compound having the structural Formula I:

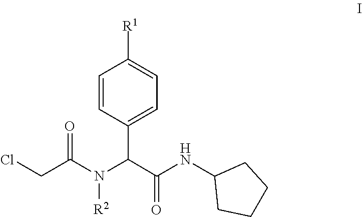

or a pharmaceutically acceptable form or an isotope derivative thereof,
wherein
R¹ is halogen or OR, wherein R is H or a C₁₋₃ alkyl; and
R² is a C₃₋₈ aliphatic or aryl group comprising 0 or 1 hetero atom selected from the group consisting of N, O, and S, wherein the C₃₋₈ aliphatic or aryl group comprises a 3- to 6-membered aliphatic or aryl ring, optionally substituted with 0-2 groups each independently selected from the group consisting of halogen, OH, and OC₁₋₃ alkyl.

In some embodiments, R¹ is halogen (e.g., F, Cl). In certain embodiments, R is —OCH₃.

In other embodiments, R² is selected from (C₀₋₂ alkylene)-(3-6 membered aliphatic ring) and (C₀₋₂ alkylene)-(5-6 membered aryl group comprising 0 or 1 hetero atom), wherein the 3-6 membered aliphatic ring and 5-6 membered aryl group are optionally substituted with 0-2 groups each independently selected from the group consisting of halogen, OH, and OC₁₋₃ alkyl.

In some embodiments, R² is selected from the group consisting of (C₀₋₂ alkylene)-cyclopropyl, (C₀₋₂ alkylene)-cyclohexenyl, (C₀₋₂ alkylene)-thiophenyl, wherein the cyclopropyl, cyclohexenyl, and thiophenyl and are optionally substituted with 0-2 groups each independently selected from the group consisting of halogen, OH, and OC₁₋₃ alkyl.

In some embodiments, R² is cyclopropyl. In certain embodiments, R² is —CH₂— thiophenyl. In other embodiments, R² is —CH₂—CH₂-cyclohexenyl.

The contemplated compound may be a compound having a structure selected from:

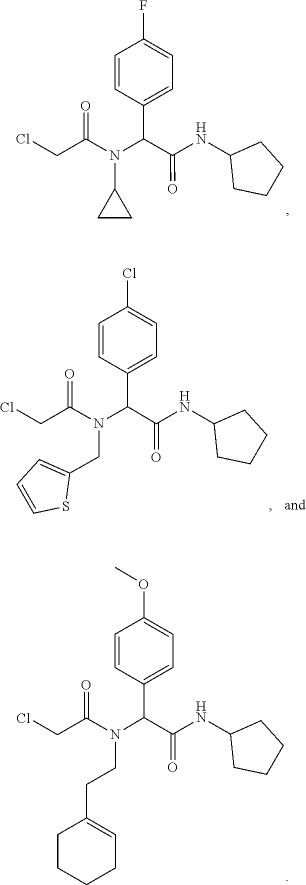 [SMILES: O=C(NC1CCCC1)C(c1ccc(F)cc1)N(C(=O)CCl)C1CC1 ; O=C(NC1CCCC1)C(c1ccc(Cl)cc1)N(Cc1cccs1)C(=O)CCl ; COc1ccc(C(C(=O)NC2CCCC2)N(CCC2=CCCCC2)C(=O)CCl)cc1]

Also provided herein is a pharmaceutical composition comprising a compound disclosed herein or a pharmaceutically acceptable form thereof, and a pharmaceutically acceptable excipient, carrier, or diluent.

In yet another aspect, the invention generally relates to a pharmaceutical composition comprising a compound disclosed herein, effective to treat or reduce one or more diseases or disorders, in a mammal, including a human, and a pharmaceutically acceptable excipient, carrier, or diluent.

In yet another aspect, the invention generally relates to a unit dosage form comprising a pharmaceutical composition disclosed herein.

In yet another aspect, the invention generally relates to a method for treating or reducing or ameliorating a disease or disorder (e.g., cancer), comprising administering to a subject in need thereof a therapeutically effective amount of a compound or a pharmaceutical composition disclosed herein.

In yet another aspect, the invention generally relates to use of a compound disclosed herein, and a pharmaceutically acceptable excipient, carrier, or diluent, in preparation of a medicament for treating a disease or disorder (e.g., cancer).

In some embodiments, the cancer is selected from the group consisting of blood cancer, breast cancer, and lung cancer.

Certain compounds designed, screened, confirmed, modified or improved according to the present invention may exist in particular geometric or stereoisomeric forms. The present invention contemplates all such compounds, including cis- and trans-isomers, R- and S-enantiomers, diastereomers, (D)-isomers, (L)-isomers, the racemic mixtures thereof, and other mixtures thereof, as falling within the scope of the invention. Additional asymmetric carbon atoms may be present in a substituent such as an alkyl group. All such isomers, as well as mixtures thereof, are intended to be included in this invention.

Isomeric mixtures containing any of a variety of isomer ratios may be utilized in accordance with the present invention. For example, where only two isomers are combined, mixtures containing 50:50, 60:40, 70:30, 80:20, 90:10, 95:5, 96:4, 97:3, 98:2, 99:1, or 100:0 isomer ratios are contemplated by the present invention. Those of ordinary skill in the art will readily appreciate that analogous ratios are contemplated for more complex isomer mixtures.

If, for instance, a particular enantiomer of a compound of the present invention is desired, it may be prepared by asymmetric synthesis, or by derivation with a chiral auxiliary, where the resulting diastereomeric mixture is separated and the auxiliary group cleaved to provide the pure desired enantiomers. Alternatively, where the molecule contains a basic functional group, such as amino, or an acidic functional group, such as carboxyl, diastereomeric salts are formed with an appropriate optically-active acid or base, followed by resolution of the diastereomers thus formed by fractional crystallization or chromatographic methods well known in the art, and subsequent recovery of the pure enantiomers.

Isotopically-labeled compounds are also within the scope of the present disclosure. As used herein, an “isotopically-labeled compound” or “isotope derivative” refers to a presently disclosed compound including pharmaceutical salts and prodrugs thereof, each as described herein, in which one or more atoms are replaced by an atom having an atomic mass or mass number different from the atomic mass or mass number usually found in nature. Examples of isotopes that can be incorporated into compounds presently disclosed include isotopes of hydrogen, carbon, nitrogen, oxygen, phosphorous, fluorine and chlorine, such as ²H, ³H, ¹³C, ¹⁴C, ¹⁵N, ¹⁸O, ¹⁷O, ³¹P, ³²P, ³⁵S, ¹⁸F, and ³⁶Cl, respectively.

By isotopically-labeling the presently disclosed compounds, the compounds may be useful in drug and/or substrate tissue distribution assays. Tritiated (³H) and carbon-14 (¹⁴C) labeled compounds are particularly preferred for their ease of preparation and detectability. Further, substitution with heavier isotopes such as deuterium (²H) can afford certain therapeutic advantages resulting from greater metabolic stability, for example increased in vivo half-life or reduced dosage requirements and, hence, may be preferred in some circumstances. Isotopically labeled compounds presently disclosed, including pharmaceutical salts, esters, and prodrugs thereof, can be prepared by any means known in the art.

Further, substitution of normally abundant hydrogen (¹H) with heavier isotopes such as deuterium can afford certain therapeutic advantages, e.g., resulting from improved absorption, distribution, metabolism and/or excretion (ADME) properties, creating drugs with improved efficacy, safety, and/or tolerability. Benefits may also be obtained from replacement of normally abundant ¹²C with ¹³C. (See, WO 2007/005643, WO 2007/005644, WO 2007/016361, and WO 2007/016431.)

Stereoisomers (e.g., cis and trans isomers) and all optical isomers of a presently disclosed compound (e.g., R and S enantiomers), as well as racemic, diastereomeric and other mixtures of such isomers are within the scope of the present disclosure.

Compounds of the present invention are, subsequent to their preparation, preferably isolated and purified to obtain a composition containing an amount by weight equal to or greater than 95% (“substantially pure”), which is then used or formulated as described herein. In certain embodiments, the compounds of the present invention are more than 99% pure.

Solvates and polymorphs of the compounds of the invention are also contemplated herein. Solvates of the compounds of the present invention include, for example, hydrates.

Any appropriate route of administration can be employed, for example, parenteral, intravenous, subcutaneous, intramuscular, intraventricular, intracorporeal, intraperitoneal, rectal, or oral administration. Most suitable means of administration for a particular patient will depend on the nature and severity of the disease or condition being treated or the nature of the therapy being used and on the nature of the active compound.

Compositions for parenteral injection comprise pharmaceutically-acceptable sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol, and the like), carboxymethylcellulose and suitable mixtures thereof, vegetable oils (such as olive oil), and injectable organic esters such as ethyl oleate. Proper fluidity may be maintained, for example, by the use of coating materials such as lecithin, by the maintenance of the required particle size in the case of dispersions, and by the use of surfactants.

These compositions can also contain adjuvants such as preservative, wetting agents, emulsifying agents, and dispersing agents. Prevention of the action of microorganisms may be ensured by the inclusion of various antibacterial and antifungal agents, for example, paragen, chlorobutanol, phenol sorbic acid, and the like. It may also be desirable to include isotonic agents such as sugars, sodium chloride, and the like. Prolonged absorption of the injectable pharmaceutical form may be brought about by the inclusion of agents which delay absorption, such as aluminum monostearate and gelatin.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically-acceptable and metabolizable lipid capable of forming liposomes can be used. The present compositions in liposome form can contain, in addition to a compound of the present invention, stabilizers, preservatives, excipients, and the like. The preferred lipids are the phospholipids and the phosphatidyl cholines (lecithins), both natural and synthetic. Methods to form liposomes are known in the art. See, for example, Prescott, Ed., Methods in Cell Biology, Volume XIV, Academic Press, New York, N.Y. (1976), p. 33 et seq.

Total daily dose of the compositions of the invention to be administered to a human or other mammal host in single or divided doses may be in amounts, for example, from 0.0001 to 300 mg/kg body weight daily and more usually 1 to 300 mg/kg body weight. The dose, from 0.0001 to 300 mg/kg body, may be given twice a day.

Solid dosage forms for oral administration include capsules, tablets, pills, powders, and granules. In such solid dosage forms, the compounds described herein or derivatives thereof are admixed with at least one inert customary excipient (or carrier) such as sodium citrate or dicalcium phosphate or (i) fillers or extenders, as for example, starches, lactose, sucrose, glucose, mannitol, and silicic acid, (ii) binders, as for example, carboxymethylcellulose, alginates, gelatin, polyvinylpyrrolidone, sucrose, and acacia, (iii) humectants, as for example, glycerol, (iv) disintegrating agents, as for example, agar-agar, calcium carbonate, potato or tapioca starch, alginic acid, certain complex silicates, and sodium carbonate, (v) solution retarders, as for example, paraffin, (vi) absorption accelerators, as for example, quaternary ammonium compounds, (vii) wetting agents, as for example, cetyl alcohol, and glycerol monostearate, (viii) adsorbents, as for example, kaolin and bentonite, and (ix) lubricants, as for example, talc, calcium stearate, magnesium stearate, solid polyethylene glycols, sodium lauryl sulfate, or mixtures thereof. In the case of capsules, tablets, and pills, the dosage forms may also comprise buffering agents. Solid compositions of a similar type may also be employed as fillers in soft and hard-filled gelatin capsules using such excipients as lactose or milk sugar as well as high molecular weight polyethyleneglycols, and the like. Solid dosage forms such as tablets, dragees, capsules, pills, and granules can be prepared with coatings and shells, such as enteric coatings and others known in the art.

Liquid dosage forms for oral administration include pharmaceutically acceptable emulsions, solutions, suspensions, syrups, and elixirs. In addition to the active compounds, the liquid dosage forms may contain inert diluents commonly used in the art, such as water or other solvents, solubilizing agents, and emulsifiers, such as for example, ethyl alcohol, isopropyl alcohol, ethyl carbonate, ethyl acetate, benzyl alcohol, benzyl benzoate, propyleneglycol, 1,3-butyleneglycol, dimethylformamide, oils, in particular, cottonseed oil, groundnut oil, corn germ oil, olive oil, castor oil, sesame oil, glycerol, tetrahydrofurfuryl alcohol, polyethyleneglycols, and fatty acid esters of sorbitan, or mixtures of these substances, and the like. Besides such inert diluents, the composition can also include additional agents, such as wetting, emulsifying, suspending, sweetening, flavoring, or perfuming agents.

Materials, compositions, and components disclosed herein can be used for, can be used in conjunction with, can be used in preparation for, or are products of the disclosed methods and compositions. It is understood that when combinations, subsets, interactions, groups, etc. of these materials are disclosed that while specific reference of each various individual and collective combinations and permutations of these compounds may not be explicitly disclosed, each is specifically contemplated and described herein. For example, if a method is disclosed and discussed and a number of modifications that can be made to a number of molecules including in the method are discussed, each and every combination and permutation of the method, and the modifications that are possible are specifically contemplated unless specifically indicated to the contrary. Likewise, any subset or combination of these is also specifically contemplated and disclosed. This concept applies to all aspects of this disclosure including, but not limited to, steps in methods using the disclosed compositions. Thus, if there are a variety of additional steps that can be performed, it is understood that each of these additional steps can be performed with any specific method steps or combination of method steps of the disclosed methods, and that each such combination or subset of combinations is specifically contemplated and should be considered disclosed.

### Additional Embodiments

In one aspect, the invention generally relates to a method for evaluating a pharmacophore or a compound for allosteric interaction with a post-translational modification (PTM) pocket on a protein. The method comprises: categorizing PTM features into sequence features (SEQ), structural and topological features (STR), and/or dynamic features (DYN); applying machine learning modeling on SEQ, STR and/or DYN features; classifying residues as allosteric PTM pockets, orthosteric residues, or others; and applying a pharmacophore or a compound to an allosteric PTM pocket via molecular modeling to determine a level of allosteric interaction between the pharmacophore or compound and the protein.

In certain embodiments, the method comprises: categorizing PTM features into SEQ, STR and DYN features; and applying machine learning modeling on SEQ, STR and DYN features. In certain embodiments, molecular modeling comprises covalent docking of the pharmacophore or compound to the allosteric PTM site. In certain embodiments, molecular modeling comprises docking of compounds to the allosteric PTM-associated pocket. In certain embodiments, categorizing PTM features comprises protein modeling. In certain embodiments, protein modeling comprises anisotropic network model (ANM) analysis, Gaussian network model (GNM) analysis, and/or principal component analysis (PCA) analysis. In certain embodiments, machine learning model comprises a random forest (RF) model. In certain embodiments, machine learning model comprises fully connected neural network (FCNN) model.

In certain embodiments, the protein is an enzyme. In certain embodiments, the enzyme is a kinase. A variety of PTMs may be analyzed using the disclosed methods. In certain embodiments, the PTM is of a type selected from the group consisting of phosphorylation, glycosylation, ubiquitylation, acetylation, sumoylation, glutathionylation, methylation, succinylation, and S-nitrosylation.

In certain embodiments, the pharmacophore or compound is a de novo pharmacophore or compound. In certain embodiments, the pharmacophore or compound is a known pharmacophore or compound.

The present invention includes confirming or validating in silico binding of a chemical compound via microscopic analysis, crystal structural analysis, and/or a biophysical assay. In certain embodiments, the disclosed method further comprises: performing a microscopic analysis, crystal structural analysis, and/or a biophysical assay to determine the level of allosteric interaction. The present invention also includes determining the efficacy of a chemical compound identified in an in vitro biological assay or in vivo in a subject. In certain embodiments, the disclosed method further comprises: performing an in vitro and/or in vivo biological assay to confirm the level of allosteric interaction.

In certain embodiments, the disclosed method further comprises: optimizing the de novo pharmacophore or compound to modify the interaction between the pharmacophore or compound with the protein, or to modify off-target effects of the pharmacophore or compound.

In another aspect, the invention generally relates to a pharmacophore or compound identified by a method disclosed herein.

In yet another aspect, the invention generally relates to a method for classifying post-translational modification (PTM) sites on a protein, comprising: categorizing PTM features into sequence features (SEQ), structural and topological features (STR), and/or dynamic features (DYN) of PTM sites; applying machine learning modeling on SEQ, STR and/or DYN features; and classifying residues in the protein as allosteric PTM pockets, orthosteric residues, or others.

The invention provides a classification of ligand-binding pockets as PTM-associated and non-PTM-associated in a protein target. Such a classification enhances attention to PTM-associated allosteric or non-PTM-associated orthosteric pockets and helps to predict the druggability and function of a ligand-binding pocket. The PTM features, e.g., SEQ, STR, and DYN, developed in this system can be used to characterize non-PTM-associated orthosteric ligand-binding pockets as well.

In yet another aspect, the invention generally relates to a system or an apparatus comprising a non-transitory computer-readable memory, a processor and a communication interface wherein the processor is connected to the non-transitory computer-readable memory and the communication interface, wherein the processor is adapted to execute instructions stored on the non-transitory computer-readable memory such that, when executed, cause the processor to perform or implement a method disclosed herein.

In yet another aspect, the invention generally relates to a compound having the structural Formula I:

In another aspect, provided herein is a compound having the structural Formula I:

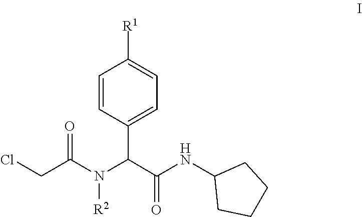

or a pharmaceutically acceptable form or an isotope derivative thereof, wherein
R¹ is halogen or OR, wherein R is H or a C₁₋₃ alkyl; and
R² is a C₃₋₈ aliphatic or aryl group comprising 0 or 1 hetero atom selected from the group consisting of N, O, and S, wherein the C₃₋₈ aliphatic or aryl group comprises a 3- to 6-membered aliphatic or aryl ring, optionally substituted with 0-2 groups each independently selected from the group consisting of halogen, OH, and OC₁₋₃ alkyl.

In yet another aspect, the invention generally relates to a pharmaceutical composition comprising a compound disclosed herein, effective to treat or reduce one or more diseases or disorders, in a mammal, including a human, and a pharmaceutically acceptable excipient, carrier, or diluent.

In yet another aspect, the invention generally relates to a unit dosage form comprising a pharmaceutical composition disclosed herein.

In yet another aspect, the invention generally relates to a method for treating or reducing a disease or disorder (e.g., cancer), comprising administering to a subject in need thereof a therapeutically effective amount of a compound disclosed herein.

In yet another aspect, the invention generally relates to use of a compound disclosed herein, and a pharmaceutically acceptable excipient, carrier, or diluent, in preparation of a medicament for treating a disease or disorder.

### EXAMPLES

The following examples are provided for the purpose of illustrating the invention, but not for limiting the scope or spirit of the invention.

### Example 1

### An Integrative Work/Low to Elucidate PTM Allosteric Regulation in Drug Design Targeting Kinase Family

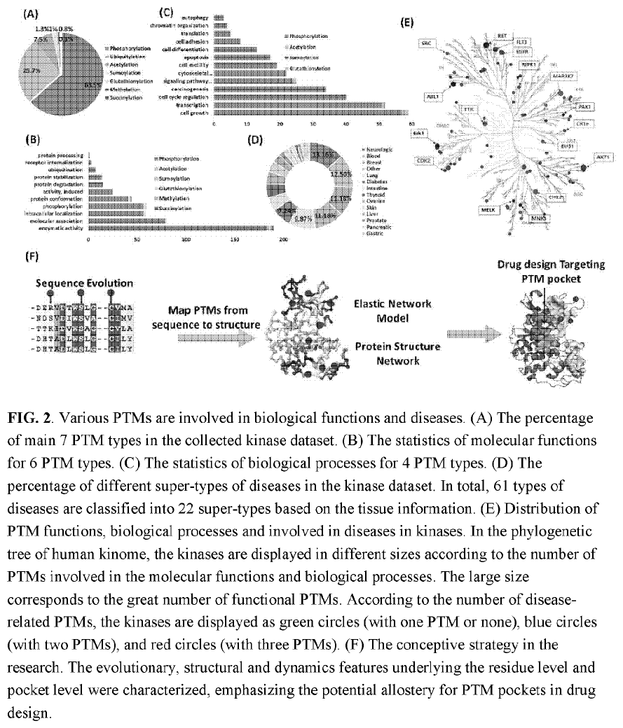

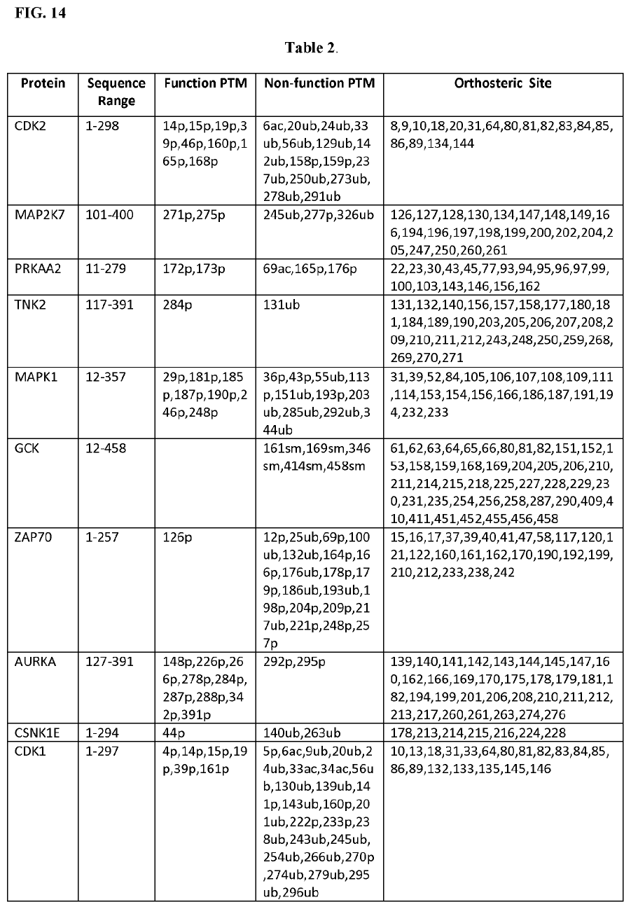

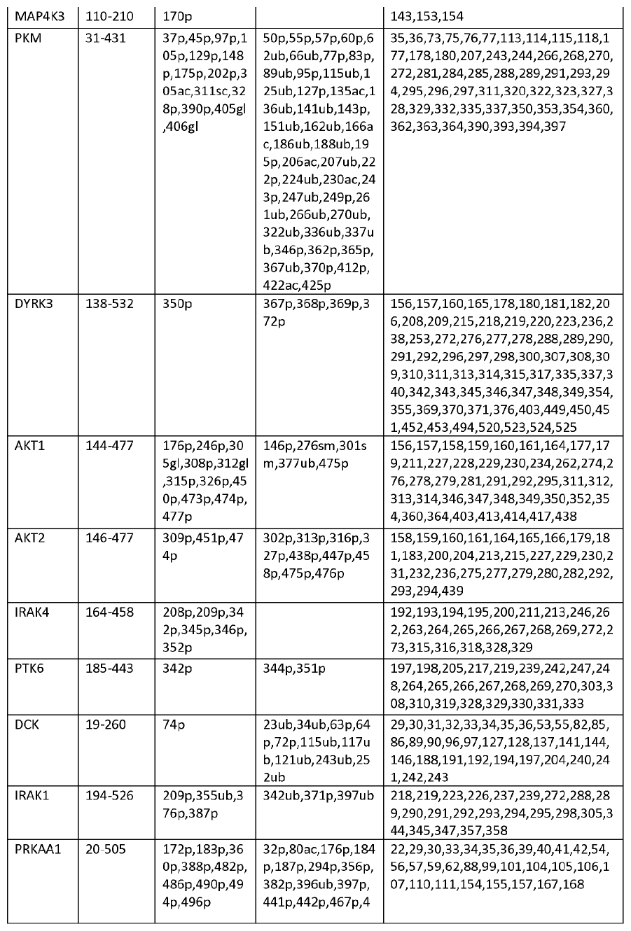

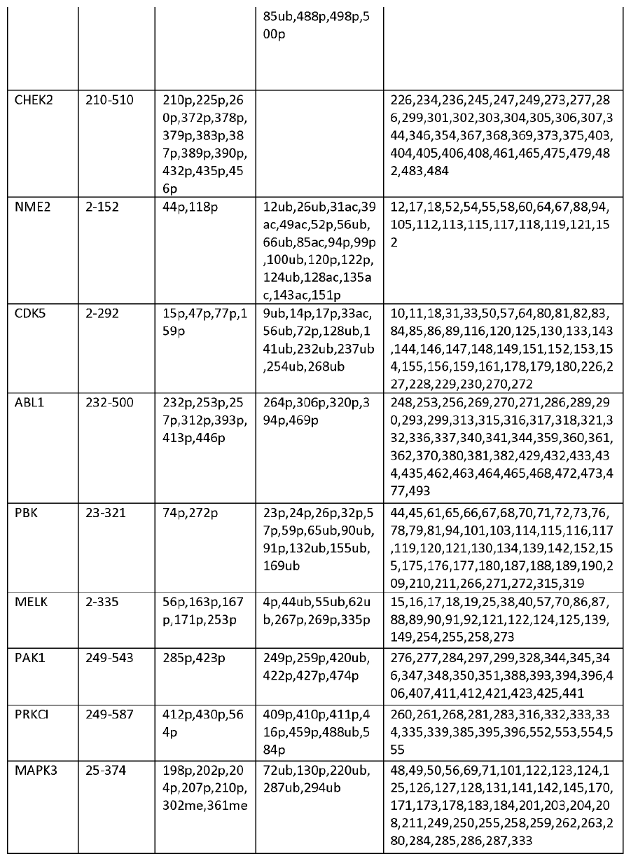

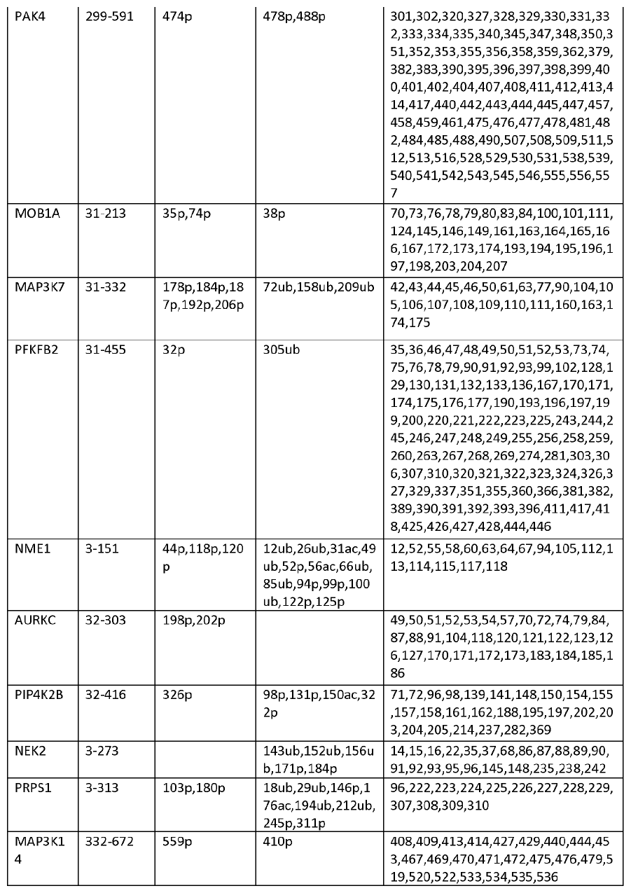

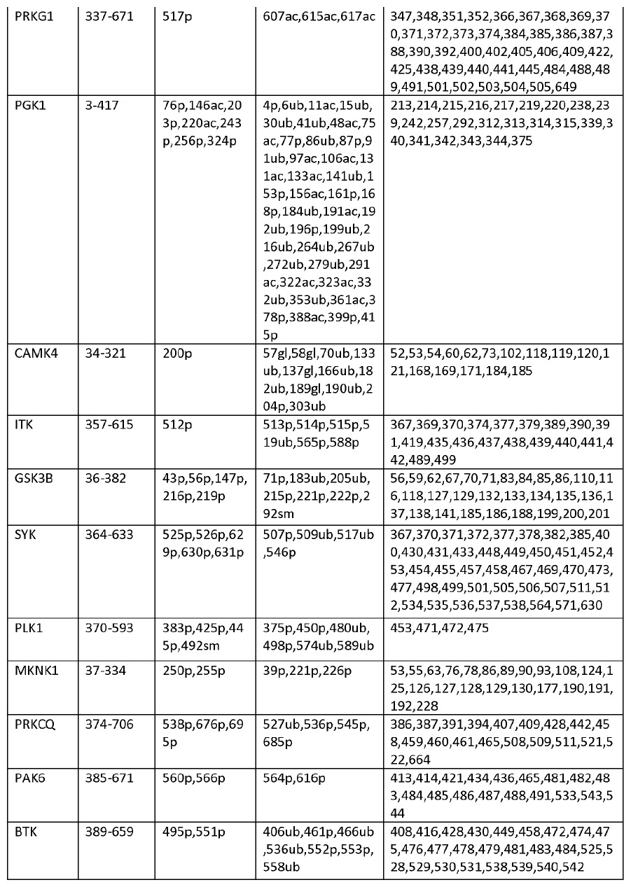

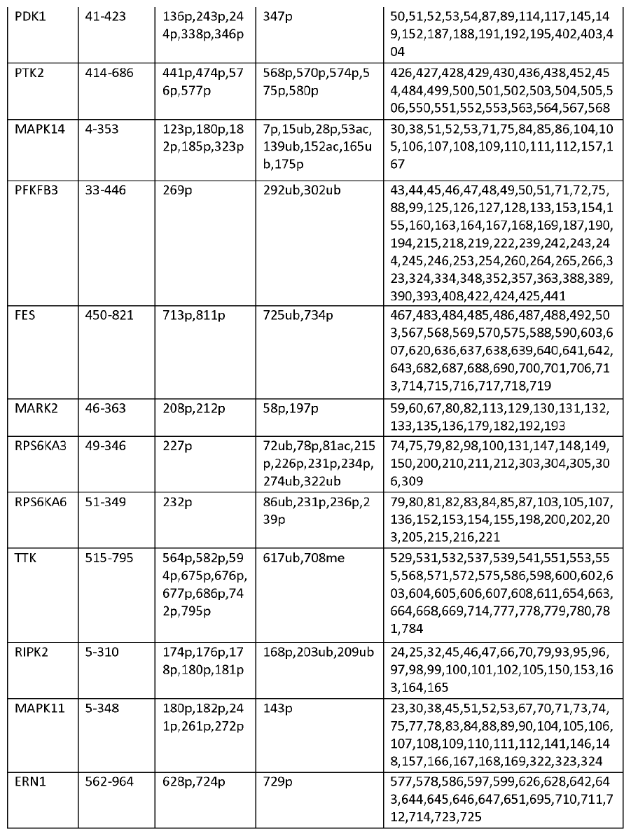

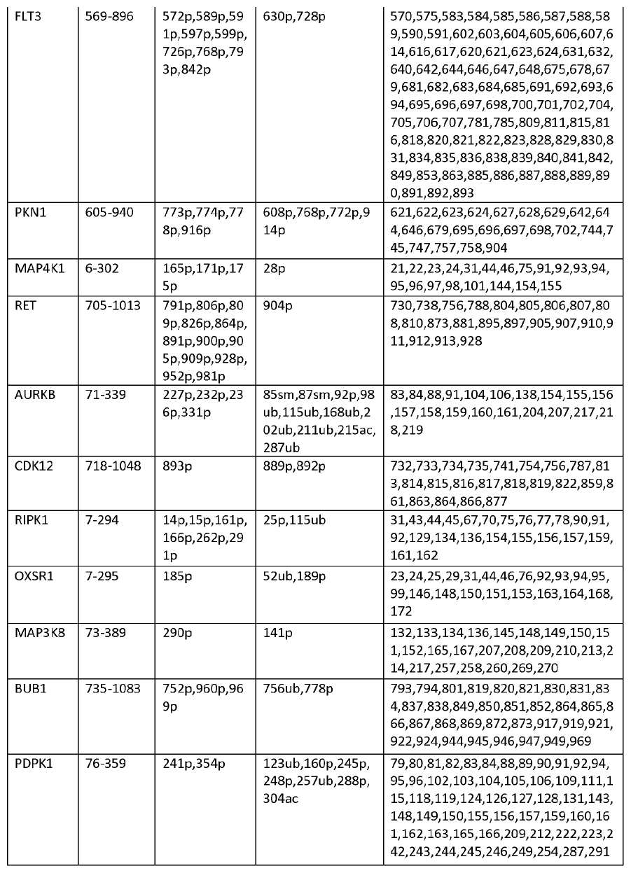

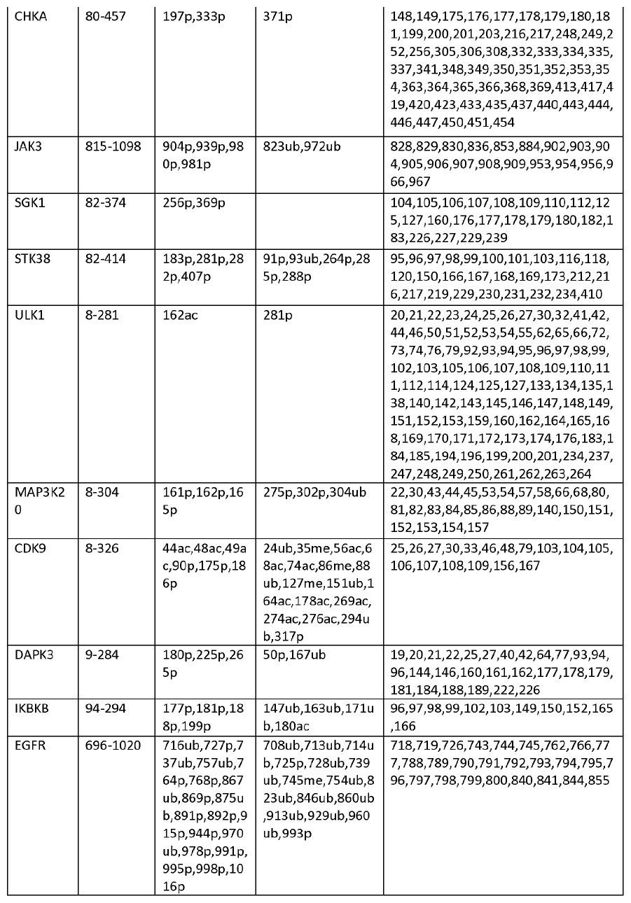

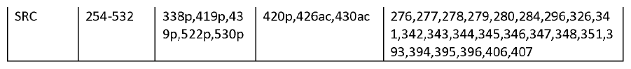

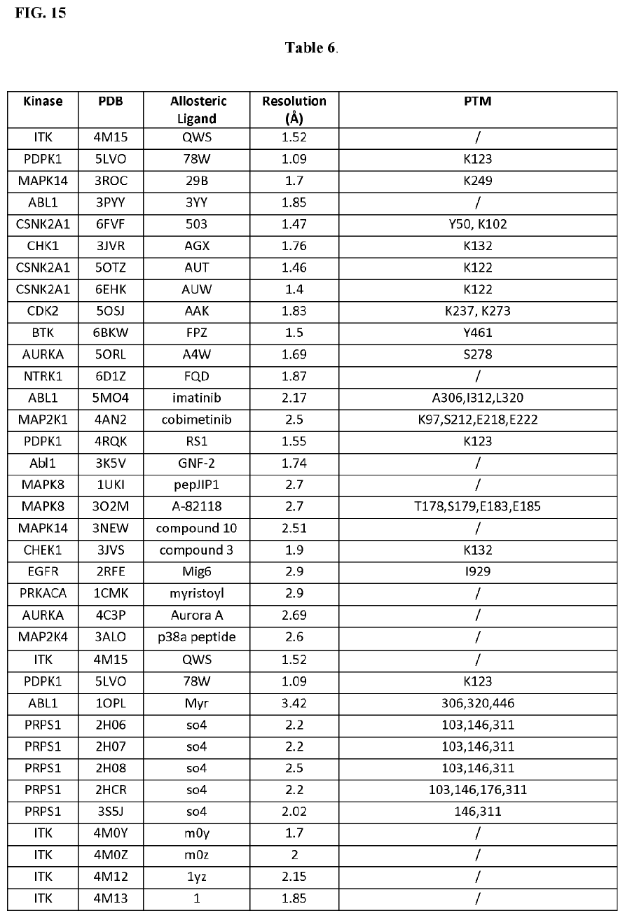

PTMs represent an important regulatory instrument that modulates the structure, dynamics, and function of proteins. As attractive drug targets in the pharmacological industry, protein kinases undergo multiple PTMs for the regulation of their activities and for cellular signaling. Through mapping PTM information from PSP database to kinase structures, 84 monomeric protein kinases in the human organism with ligand binding site (also recognized as orthosteric site) information were collected, including 836 PTM sites (see, Table 2, FIG. 14). The major classes of the PTMs included those that were most widely studied (FIG. 2A), such as phosphorylation (531 sites) of serine, threonine, and tyrosine; ubiquitylation (215 sites) of lysine; and acetylation (63 sites) of lysine. The minor classes included sumoylation (11) of lysine; glutathionylation (8) of threonine and serine; methylation (7) of lysine and arginine; and succinylation (1) of lysine. The reported molecular functions were mostly affected by the phosphorylations, including those of enzymatic activity, molecular association, and intracellular localization (FIG. 2B). Correspondingly, the most reported biological processes involved cell growth, transcription, cell cycle regulation and carcinogenesis (FIG. 2C).

Perturbed signaling is the most common cause of uncontrolled cancer-triggering cell growth and proliferation. Thus, the significant involvement of diseases of PTMs is illustrated by their impacts on cell functions and processes. Based on the disease-related information of PTMs curated from the literature in the PSP database, the most involved diseases included neurological diseases, cancers (of the blood, breast, and lung), and diabetes (FIG. 2D). To date, the PTMs in the serine/threonine AGC protein kinase (AKT1) has the largest number of associated diseases in the PTMD database². Dual phosphorylation on residues T308 and S473 is required for its complete activation, which is crucial for cell cycle regulation and carcinogenesis. The altered phosphorylation levels on both residues have been reported to be involved in numerous cancers, neurological diseases, and type 2 diabetes²³. Thus, targeting kinases to disrupt PTMs involved in diseases could achieve precision medicine, whereas limited knowledge of PTM dynamics in the allosteric regulation of proteins limits the understanding for targeting this unexplored biological space.

The distribution of PTMs with regulatory roles and involved in diseases across the human kinome was determined as shown in FIG. 2F Although PTMs are involved in specific regulation of proteins in cellular signaling, systematic dynamics analyses of PTM sites are still lacking. Herein, the structural variations of the experimental structural ensembles were analyzed in the representative kinase of each family, including cyclin-dependent kinase—CDK2, AKT1, the non-receptor tyrosine kinase—c-Src, p21-activated kinase—PAK1, checkpoint kinase-CHK2 and receptor interacting protein kinase—RIPK1. By mapping PTMs onto 84 kinase structures, it was found that most PTMs prefer into the loop regions. In the strategy of the research (FIG. 2F), sequence information, structural topological and dynamics information were calculated using multiple sequence alignment and coarse-grained network models. Using statistical analyses, the specific sequences, structural topologies and in particular the dynamics signatures for PTM sites were identified. Furthermore, PTM pockets were designated and the potential allostery of PTM pockets were evaluated in drug design, to enlarge the targetable space in kinase families.

### The Dynamics Plasticity of PTM Sites Encodes the Intrinsic Dynamics of Kinase Structure

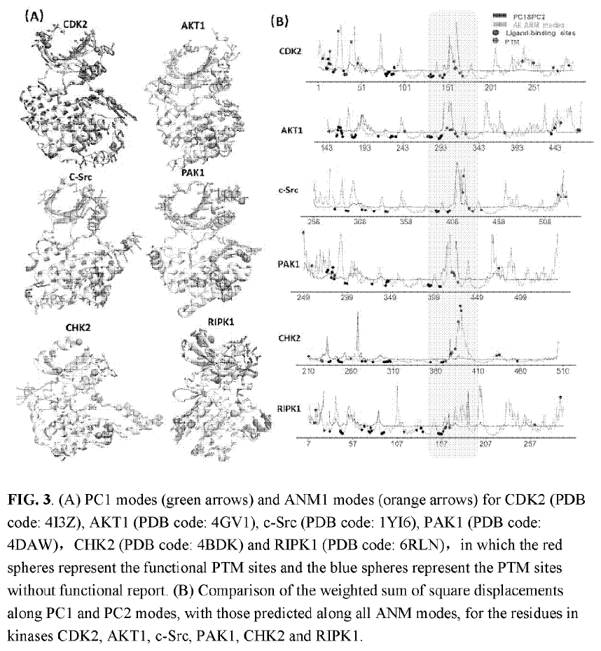

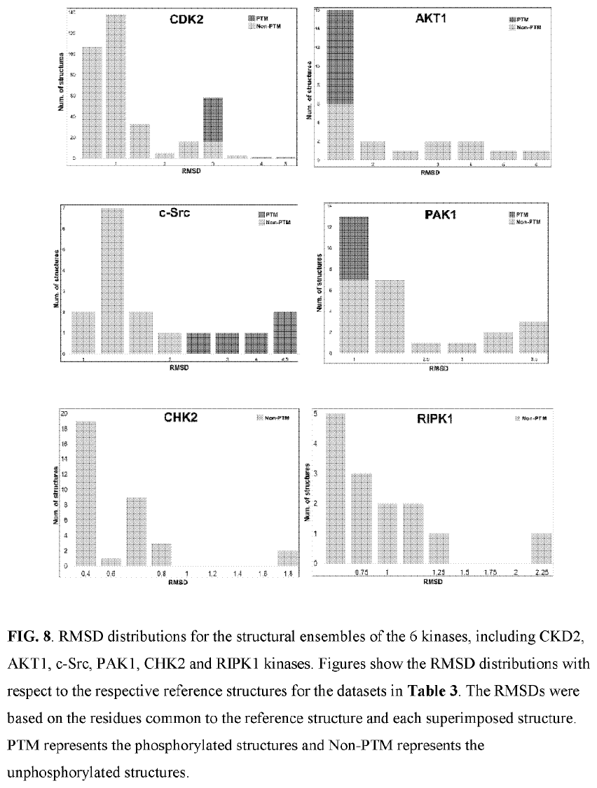

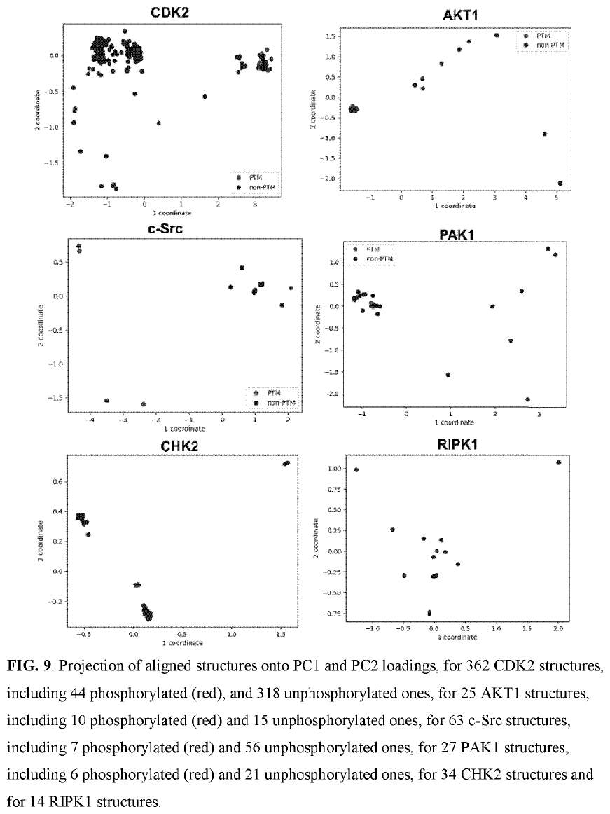

Nowadays, tremendous crystal structures of kinase family, with various small molecules or protein counter partners, with or without phosphorylated, have been resolved. To characterize the conformational variations, the X-ray crystallographic structures of CDK2, AKT1, c-Src, PAK1, CHK2, and RIPK1, as representatives of each kinase family, were collected in Table 3. The corresponding distributions of RMSDs (FIG. 8) were approximately 0-6 Å in each kinase dataset, with most entities clustered in the <2 Å range. Using principal component analysis (PCA), the distributions of the structural ensembles along the first two PC modes (FIG. 9) showed that the phosphorylated kinase structures were clustered. The PC1 modes, explaining the principal variations in these experimental structural ensembles (listed in Table 4), mainly referred to the anticorrelated movements of the N-lobe and the activation loop in the C-lobe (FIG. 3A, green arrows). These movements corresponded to a separation of >4 Å between the two distant conformers (except for CHK2 and RIPK1) along the PC1 axis (FIG. 9), which accounted for the majority of variances in the activation loop observed in the datasets. In CHK2, the resolved structures only captured the activation loop in the open state (FIG. 3A) within a remarkable dimeric arrangement²⁴, whereas in RIPK1, the activation loop was only in the closed state, packing tightly to the αC helix and resulting in an inactive state (FIG. 3A)²⁵. Thus, the PC modes for both kinases could not identify the variations in the activation loop. In addition, the C-terminal of c-Src, incorporating Y522 and Y530 as the phosphorylation sites, also displayed large conformational changes in the PC1 mode.

TABLE 4
Overlap between PCA and ANM modes

| | | ANM modes | | | | | | |
|---|---|---|---|---|---|---|---|---|
| PCA modes | | | | | | | | All |
| (fractional | | | | | | 20 ANM | 100 | ANM |
| contribution) | | ANM1 | ANM2 | ANM3 | | modes | ANM modes | modes |
| CDK2 | PC1 (90.85) | 0.16 | 0.02 | 0.32 | PC1 | 0.69 | 0.83 | 1.00 |
| | PC2 (3.24) | 0.12 | 0.23 | 0.03 | 2PCs | 0.73 | 0.83 | 0.995 |
| AKT1 | PC1 (78.11) | 0.31 | 0.06 | 0.01 | PC1 | 0.49 | 0.72 | 0.997 |
| | PC2 (9.20) | 0.10 | 0.04 | 0.15 | 2PCs | 0.34 | 0.57 | 0.999 |
| PAK1 | PC1 (65.60) | 0.41 | 0.44 | 0.13 | PC1 | 0.76 | 0.87 | 1.00 |
| | PC2 (11.74) | 0.34 | 0.12 | 0.06 | 2PCs | 0.58 | 0.72 | 1.00 |
| c-Src | PC1 (94.35) | 0.15 | 0.22 | 0.18 | PC1 | 0.68 | 0.82 | 0.996 |
| | PC2 (5.25) | 0.03 | 0.17 | 0.07 | 2PCs | 0.53 | 0.73 | 0.994 |
| CHK2 | PC1 (60.96) | 0.20 | 0.09 | 0.10 | PC1 | 0.75 | 0.77 | 1.00 |
| | PC2 (23.49) | 0.19 | 0.14 | 0.18 | 2PCs | 0.86 | 0.84 | 1.00 |
| RIPK1 | PC1 (49.78) | 0.15 | 0.20 | 0.14 | PC1 | 0.35 | 0.60 | 1.00 |
| | PC2 (19.14) | 0.01 | 0.37 | 0.16 | 2PCs | 0.42 | 0.68 | 0.999 |

To compare conformational changes from experimental structural ensembles to those with predicted dynamics, the physics-based ANM models for the kinase structures were calculated and compared with the top-ranking PC modes. The overlap values between the top-ranking two PC modes and first 3, 20, and 100 ANM modes, and all ANM modes, were respectively listed in Table 4. Compared with the local variations deciphered by the top-ranking PC modes, the low-frequency ANM modes mainly captured the collective motions, resulting in the not high overlap values. The first ANM modes (FIG. 3A, orange arrows) mainly referred to the twisting motions between the N-lobe and C-lobe, neglecting the detailed local variations in structures. Although the correlations were not high for the top-ranking PC modes and low-frequency ANM modes, the overlap values between the PC modes and first 20, 100 and all ANM modes reached 0.6-1 (Table 4), suggesting that the conformational variations observed in experimental ensembles could be explained by theoretical dynamics calculated by ANMs using single structures. These observations supported the possibility that “functional changes in structures” predominantly obeyed “structure-encoded preferences”.

Through mapping square displacements of residues along the PC modes (including PC1 and PC2) and all ANM modes, it was shown that the activation loops displayed high flexibility in the PC modes and all ANM modes (blue box in FIG. 3B). The activation loops, enriched in PTM sites, were representative conformational variations between the active (phosphorylated in the open state) and inactive state of the kinase, indicating the regulations induced by phosphorylation. The exception was CHK2, in which the unique activation loop in the monomeric form only displayed extremely high flexibility in the ANM modes. In contrast to the high flexibility of PTM sites (red dots in FIG. 3B), the ligand-binding sites (as the orthosteric sites, black dots in FIG. 3B) were located at the minima of these profiles in both the PC and ANM modes. All ANM modes could, therefore, capture structural variations in the experimental ensembles, so subsequently calculated were the “sequence-structure-dynamics” features for PTM residues and orthosteric residues for the dataset in Table 2 (FIG. 14).

### Dynamics Features of PTM Sites Bridge Kinase Structure and Allosteric Regulation

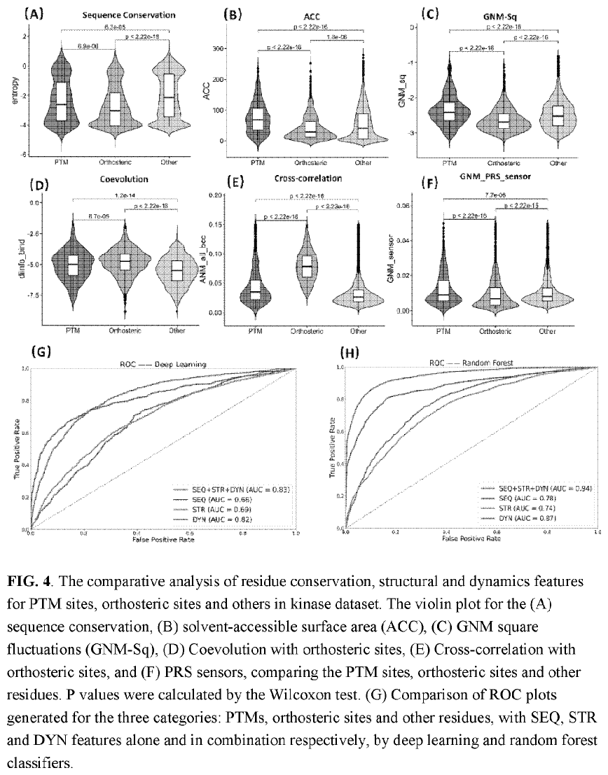

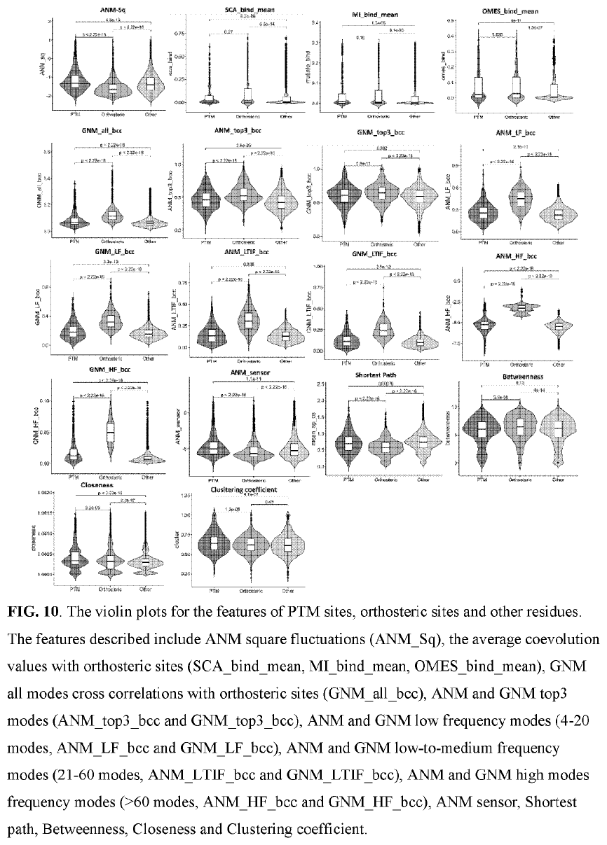

The sequence variability and structural dynamics usually go hand in hand. However, the relationship between sequence evolution and structural dynamics for PTM sites remains to be investigated. Based on the violin plot for 84 kinases, the orthosteric residues possessed the most conserved features (the least entropy values), with the PTM sites ranked as second (FIG. 4A). The high conservation of orthosteric sites was essential for the enzymatic catalysis and ligand binding, whereas certain conservation of PTM sites indicated their crucial roles in protein functions and biological processes. A systematic analysis of phosphorylation, acetylation, and ubiquitination sites showed that the evolutionary conservation of PTMs within domain families could pinpoint the regulatory hotspots²⁶. Structurally, the PTM sites possessed the largest solvent-accessible surface area (ACC) values (FIG. 4B), suggesting their locations in the convex surface and their accessibilities for modifying enzymes to add the modifications²⁷. The orthosteric residues possessed the lowest ACC values. Correspondingly, PTM sites had the highest degrees of flexibility, corresponding to the largest mean-square fluctuations, both in GNM and ANM models (FIG. 4C and FIG. 10). In contrast, the orthosteric residues possessed the least flexibility, acting as hinges for supporting the mechanics of proteins

From sequence evolutionary, the PTM sites also possessed high values of coevolution with the orthosteric sites when using different coevolution methods (FIG. 4D and FIG. 10), indicating robust coevolutionary coupling between PTMs and orthosteric sites. The coupled evolutionary changes in pairs of positions along the amino acid sequence provided allosteric inferences regarding the dynamics²⁸. Concerning the dynamics cross-correlation values in both the ANM and GNM (FIG. 4E and FIG. 10), the PTM sites possessed higher coupling cross-correlations with the orthosteric sites, in all modes, and lower and higher frequency modes, indicating their potential allosteric regulatory roles for enzymatic activity and ligand binding. This is consistent with previous research showing that the allosteric sites had high coupling correlations with active sites using GNM models^{22c}. In Hsp70 proteins, the dynamics and co-evolutionary correlations acted as synchronizing forces to enable efficient and robust allosteric regulations^{27,29}. In addition, the PTM sites had the potential for serving as sensors for external perturbations (FIG. 4F) and possessed the shortest paths to the orthosteric residues (FIG. 10). Both features are determinants for allosteric PTM sites in the regulation of protein function.

Concerning the topological descriptors (FIG. 10), the orthosteric sites achieved higher betweenness values, indicating their possible involvement in the shortest communication paths. In contrast, the PTM sites possessed high closeness and clustering coefficient values, indicating that although the PTM sites were not in the center of residue networks, there were close contacts between their neighboring residues. These locations generally corresponded to the protein pocket, cleft, and cavity regions, and indicated the high efficiency communication through these nodes³⁰.

Computational tools have been developed for specific PTM predictions, mainly using sequence and structural features. Herein, firstly introduced were the dynamics features underlying PTMs in kinases. The comprehensive features were categorized into sequence features (SEQ), structural features (STR), and dynamics features (DYN), as shown in Table 5.

TABLE 5
Summary of features in each of the three categories Feature name

| Feature category | Feature name |
|---|---|
| Sequence | 1. Residue identity (20 standard amino acids) 2. |
| information | Conservation 3. Co-evolution |
| Structural | 1. Solvent accessibility 2. Node centralities calculated |
| information | using weighted PSN |
| Dynamics | 1. b-factor 2. Mean-squared fluctuations by ENMs |
| information | 3. Coupling cross-correlation values by ENMs |
| | 4. Perturbation response scanning |

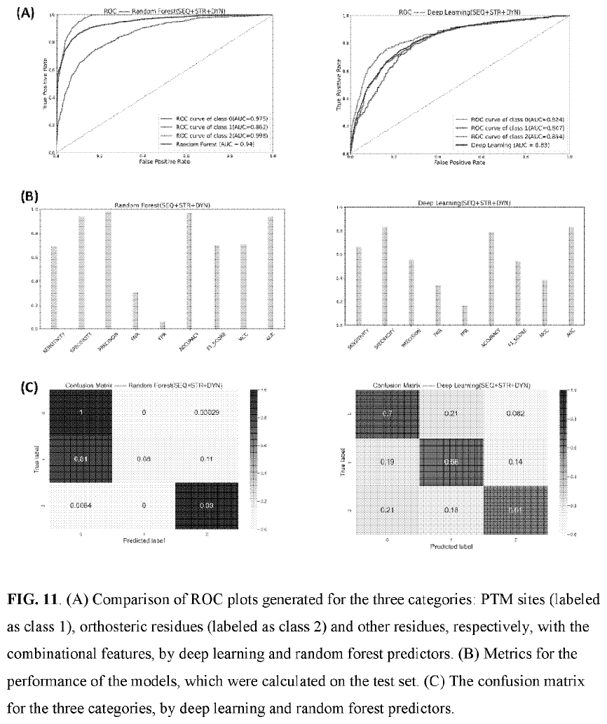

Two machine learning models to classify the PTM sites, orthosteric sites and others were reported, involving the random forest (RF) model and deep learning model with fully connected neural network (FCNN). The results from testing the predictors on SEQ, STR or DYN features exclusively, and on their combination, were shown in receiver operating characteristic (ROC) plots (FIGS. 4G and 4H). In both models, the combination predictions showed better accuracy than the single features alone, with AUC values of 0.83 and 0.94 in the deep learning and random forest, respectively. Closer examination showed that the DYN classifier alone outperformed the SEQ and STR classifiers, emphasizing the importance of dynamics in PTM characterization. The detailed information is listed in FIG. 11.

### Dynamics Features Underlying PTM Pockets Highlight the Allosteric Potential in Drug Design

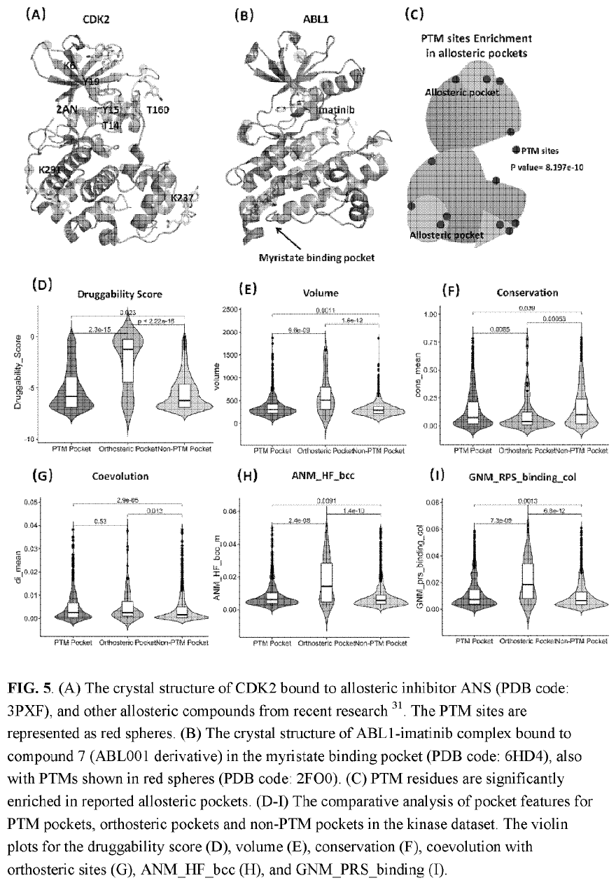

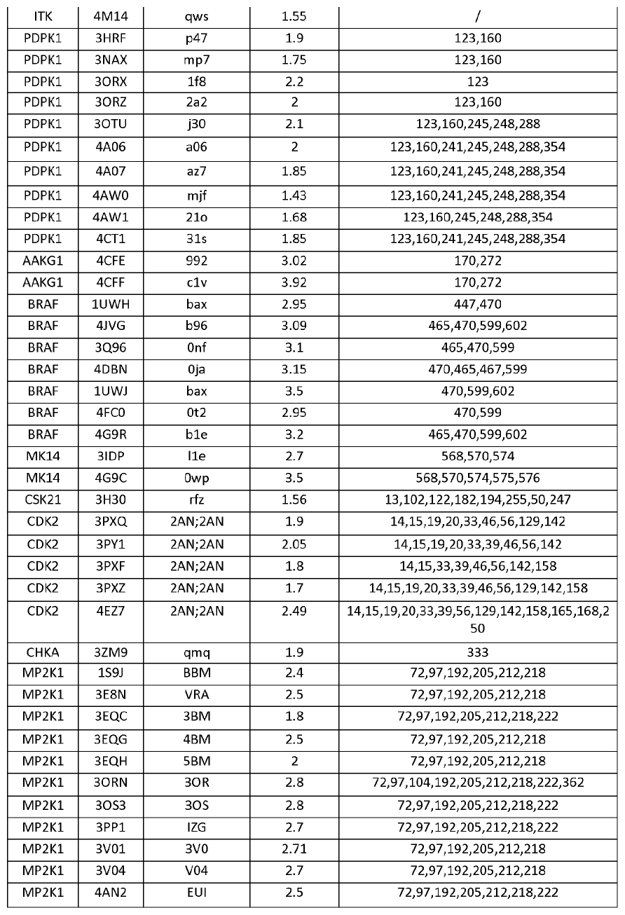

The dynamics features underlying PTM sites indicated high potential allosteric sites for drug design. Interestingly, it is worth noting that PTM sites frequently appeared in the identified allosteric pockets. In CDK2 (FIG. 5A), the allosteric inhibitor, 2AN, bound in a large pocket away from the ATP site, surrounded by phosphorylation sites at T14 and Y15 in the P loop, and phosphorylation at Y159 and T160 in the activation loop³². In addition, a handful of allosteric compounds using the FragLites approach³¹ were also gathered in the PTM regions, using alignment with the same structure (FIG. 5A). In ABL1 kinase, the allosteric inhibitor, ABL001 (asciminib), bound to the myristoyl pocket and induced the formation of an inactive conformation (FIG. 5B) 33. ABL001 has recently been reported positive in phase III clinical trial for chronic myeloid leukemia and acute lymphoblastic leukemia. The combinations of ABL001 with ATP-competitive tyrosine kinase inhibitors can suppress the emergence of mutation resistance³⁴. By mapping PTM sites to the collected kinase structures bound with allosteric compounds (Table 6, FIG. 15), it was shown that PTM sites were significantly enriched in the identified allosteric pockets (P value=8.197e-10 using the hypergeometric test, FIG. 5C), highlighting the allosteric potential for targeting pockets within PTMs.

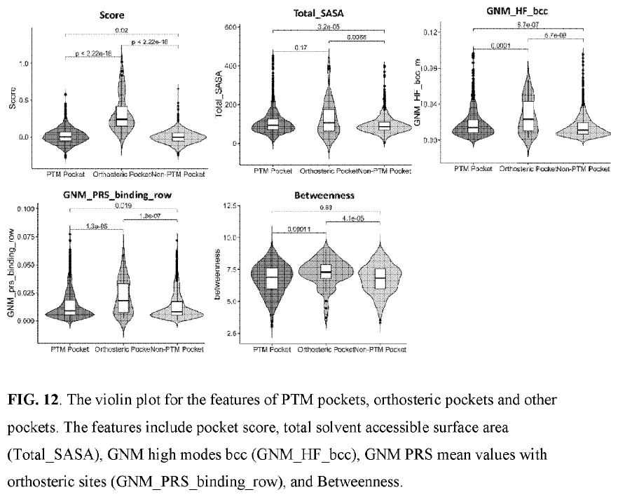

Next categorized were the pockets predicted by Fpocket into PTM pockets (incorporating PTM sites by expanding two residues of the pocket residues), non-PTM pockets, and orthosteric pockets, and subsequently analyzed these pockets based on sequence, structural topology and dynamics features. Except for the absolute advantage for orthosteric pockets (FIG. 5D and FIG. 12), PTM pockets ranked better than non-PTM pockets in the pocket score and druggability score, indicating their potential draggabilities. The ranking situation was the same in the pocket volume and solvent accessible area features (FIG. 5E and FIG. 12). From sequence evolution, the pocket conservation was evaluated by averaging the entropy values of all residues in the pocket. Except for the most conserved orthosteric pockets, PTM pockets had statistically conserved characteristics, when compared with non-PTM pockets (FIG. 5F). The conservation score has been shown to be valuable for active site detection and functional site prediction in certain datasets³⁵.

In addition, PTM pockets possessed statistically higher evolutionary coupling (coevolution, FIG. 5G) and dynamics coupling (dynamics cross correlations, FIG. 5H) with orthosteric pockets, when compared to non-PTM pockets. The higher dynamics cross correlations were observed in the high frequency modes in both ANM and GNM (FIG. 5H and FIG. 12), indicating the high allosteric potential for PTM pockets. In addition, the sensor and effectiveness values for the response upon perturbing the orthosteric pockets were higher for PTM pockets (FIG. 5 and FIG. 12), also acting as determinants for allostery. Concerning network topologies, the orthosteric pockets possessed the largest centrality values, whereas PTM pockets had no significant differences compared with non-PTM pockets (FIG. 12). Overall, PTM pockets were characterized in terms of certain conservation and higher coupling with the orthosteric residues. Thus, these features together with the high druggability score highlighted the allosteric target nature of PTM pockets.

A Covalent Inhibitor was Identified Targeting the PTM-Associated Pocket in c-Src Kinase

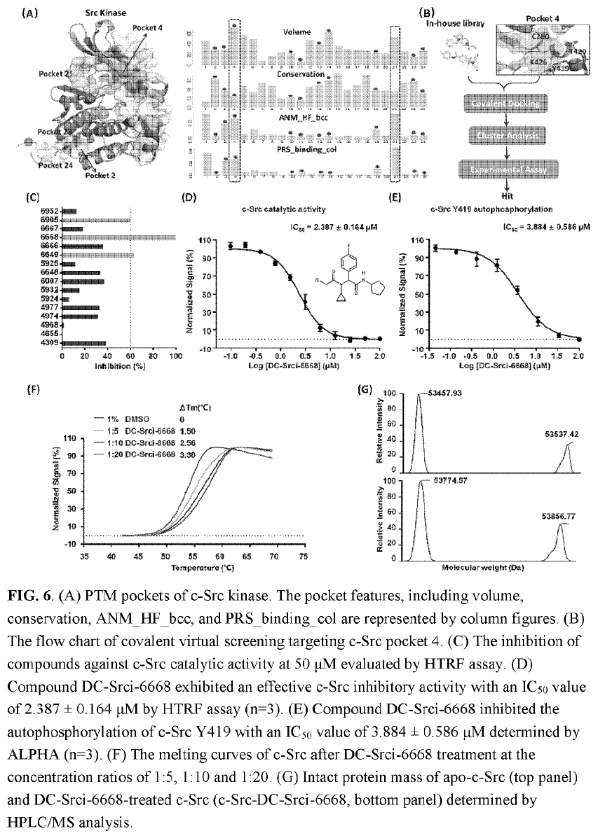

Based on the allosteric evaluation for PTM pockets, c-Src kinase was selected as an example for subsequent exploration of PTM inspired drug design. Computational studies have revealed the molecular activation mechanism of c-Src kinase^{10a,36}, enriching understanding of PTM regulation. To design inhibitors targeting PTM sites, the PTM pockets in c-Src (FIG. 6A) were checked and PTM pocket 4 was identified, similar to the 2AN binding pocket in CDK2, representing a high potential targetable pocket. PTM pocket 4, adjacent to the ATP binding site, incorporated the phosphorylation site at Y419 and acetylation site at K426. Recent studies established the critical importance of C280, also in pocket 4, as a substrate site for H₂O₂-mediated sulfenylation in response to NADPH oxidase-dependent signaling³⁷. Through feature analyses, PTM pocket 4 was shown to possess a large volume and moderate conservation. In addition, PTM pocket 4 had high coupling dynamics correlations with the ATP binding site and high response upon perturbation at the ATP site. On the other hand, PTM pockets 23/24, similar to the myristate-binding pocket in c-Abl, have been shown to bind myristate by NMR experiments³⁸. PTM pockets 23/24 could merge into one pocket with conformational fluctuations in the C-terminal tail. This pocket, distant from the ATP binding site and incorporating the phosphorylation sites at Y522 and Y530, represented as an allosteric PTM pocket. Attributed to the high flexibility of the C-terminal tail, PTM pocket 4 was finally selected for subsequent drug design.

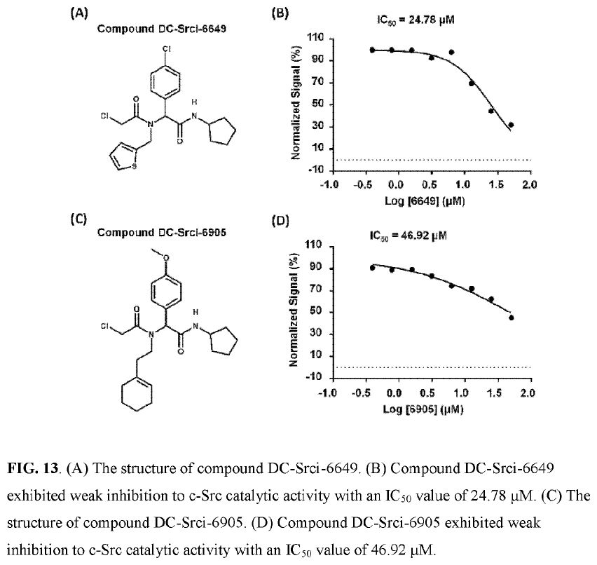

Recent studies have successfully proved the inhibitor design by modifying the electrophile to covalently interact with the cysteine in c-Src kinase³⁹. Hence, it is feasible to identify covalent inhibitors by targeting C280 in PTM pocket 4, to precisely regulate the phosphorylation of Y419 in c-Src. Based on this, covalent docking was performed against an in-house compound library, consisting of 720 compounds with reactive chemical groups. After score ranking and cluster analysis (FIG. 6B), 16 compounds were selected and purchased from ChemDiv (San Diego, CA, USA) for subsequent biochemical evaluations. Although IC₅₀ values are time-dependent for covalent inhibitor and cannot be used to fully characterize the true binding affinity, they are also recognized as an indicator for preliminary evaluating the biological activity of covalent compounds. The homogeneous time resolved fluorescence (HTRF) assay was then conducted to determine the compound inhibitory activity, using the HTRF KinEASE-TK kit (Cisbio, Bedford, MA, USA). Three compounds showed greater than 60% inhibition to c-Src catalytic activity at a concentration of 50 μM after 120 min incubation (FIG. 6C). In the determination of IC₅₀ values, the lead compound DC-Srci-6668 was identified with an effective c-Src inhibitory activity after 120 min incubation (IC₅₀=2.387±0.164 μM) (FIG. 6D and FIG. 13). Subsequently, the Amplified Luminescent Proximity Homogeneous Assay (ALPHA) showed that compound DC-Srci-6668 inhibits the autophosphorylation of c-Src Y419 with an IC₅₀ value of 3.884±0.586 μM after 90 min incubation (FIG. 6E), indicating the inhibitory activity could be attributed to the disturbed phosphorylation in PTM pocket 4 of c-Src.

Biophysical experiments were further executed to validate the binding mode of lead compound DC-Srci-6668. Protein thermal shift (PTS) assay showed that compound treatment led to increased melting temperature (Tm) of c-Src in a dose-dependent manner (positive Tm shifts of 1.50° C., 2.56° C. and 3.30° C. with concentration ratios at 1:5, 1:10 and 1:20 respectively, FIG. 6F), conforming the direct binding between c-Src and DC-Srci-6668. In addition, high-performance liquid chromatography-mass spectrometry (HPLC/MS) analysis was conducted to determine the molecular weight (MW) of apo-c-Src and DC-Srci-6668-treated c-Src (c-Src-DC-Srci-6668) respectively. It showed that after DC-Srci-6668 treatment, the MW of c-Src was increased by 316.64 Da (FIG. 6G), consistent with the expected MW increase after a single DC-Srci-6668 compound covalently bound through nucleophilic substitution (316.34 Da). Overall, these results demonstrated that compound DC-Srci-6668 directly and covalently reacted with c-Src to disrupt the phosphorylation in PTM pocket 4.

Complex Crystal Structure Confirms the Inhibitory Mechanism of DC-Srci-6668 for Targeting c-Src PTM Pocket

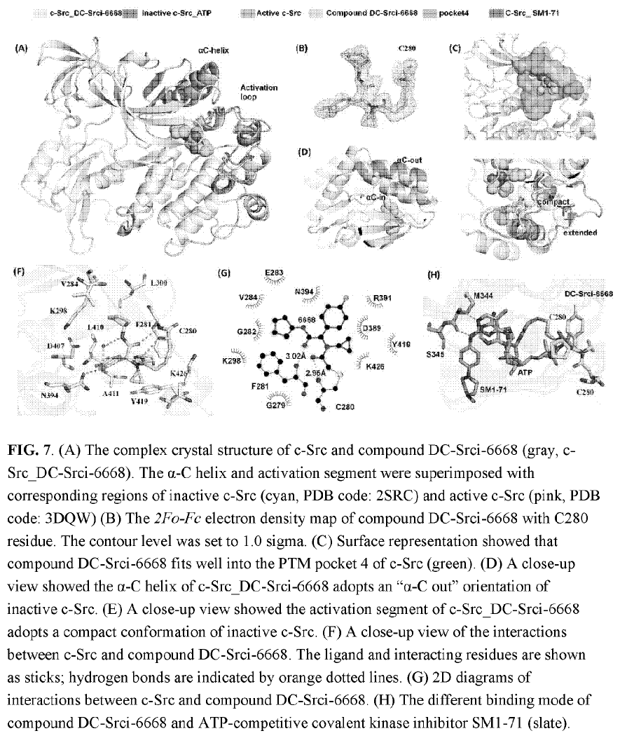

To gain further insight into the binding mode and inhibition mechanism of compound DC-Srci-6668, the crystal structure of c-Src in complex with DC-Srci-6668 was solved at 1.9 Å resolution (FIG. 7A, and data statistics were listed in Table 1, PDB code: 7ELU). The clear and relatively intact electron density map (2Fo-Fc at 1.0a, FIG. 7B) confirmed the covalent binding between DC-Srci-6668 and C280 residue of c-Src. The surface representation revealed that DC-Srci-6668 fit well into the PTM pocket 4 (FIG. 7C), consistent with the covalent docking and biophysical results. Compared to both inactive and active conformations of c-Src (PDB code: 2SRC and 3DQW, respectively)⁴⁰, the regulatory αC helix (S306-L320) in c-Src_DC-Srci-6668 complex crystal structure corresponded to the “αC-out” conformation in the inactive (FIG. 7D). Besides, the activation segment (D410-E435) in c-Src_DC-Srci-6668 represented a compact conformation with Y419 unphosphorylated, similar to the inactive c-Src (FIG. 7E). In contrast, the activation segment displayed an extended open conformation with phosphorylated Y419 in the active state. These observations supported that compound DC-Srci-6668 binds to the PTM pocket 4 of inactive c-Src and disrupts the autophosphorylation of Y419, thereby capturing c-Src in inactive state and inhibiting c-Src activation.

Concerning the detailed binding modes (FIG. 7F and FIG. 7G), the α-chloroacetyl warhead of compound DC-Srci-6668 covalently bound to C280 of pocket 4 through nucleophilic substitution. Besides, DC-Srci-6668 was wrapped by hydrophobic residues, including F281, V284, L300, L410, A411 and Y419 residues. The side-chains of K298, R391 and K426 residues also contributed to partial hydrophobic interactions as well. Moreover, multiple hydrogen bond interactions further supported and stabilized the combination of DC-Srci-6668. The carbonyl group of cyclopropylacetamide formed a hydrogen bond with C280. The carbonyl group of cyclopentylacetamide formed another hydrogen bond with F281. Besides, the amino group of cyclopentylacetamide bridged to N394 through two water molecules. Compared to the crystal structure of c-Src in complex with ATP-competitive covalent inhibitor SM1-71, compound DC-Srci-6668 represented a totally different binding mode (FIG. 711). In addition to reacting with C280 residue, SM1-71 occupied the adenine binding site of ATP and interacted with the hinge residues (E342-G347). Whereas, DC-Srci-6668 merely embedded in PTM pocket 4, which obviously has no overlap with the adenine binding site of ATP. Taken together, compound DC-Srci-6668 is a covalent inhibitor of c-Src targeting the PTM pocket, which selectively binds to the inactive state. By disturbing the autophosphorylation of Y419, compound DC-Srci-6668 precisely regulates PTM and prevents the activation of c-Src.

TABLE 1
Data Collection and Structure Refinement Statistics

| Dataset | Src-DC-Srci-6668 | |
|---|---|---|
| Wavelength | 0.97867 | |
| Resolution range | 46.16-1.90 | (1.96-1.90) |
| Space group | P 21 21 21 | |
| Unit cell | 51.189 82.91 106.623 90 90 90 | |
| Total reflections | 454988 | (36651) |
| Unique reflections | 35550 | (3029) |
| Multiplicity | 12.8 | (12.1) |
| Completeness (%) | 97.63 | (84.23) |
| Mean I/sigma(I) | 22.77 | (2.0) |
| Wilson B-factor | 20.38 | |
| R-merge | 0.032 | (0.384) |
| R-meas | 0.115 | (1.361) |
| CC1/2 | 0.991 | (0.663) |
| CC* | 0.998 | (0.893) |
| Reflections used in | 35542 | (3029) |
| refinement | | |
| Reflections used for R-free | 1732 | (142) |
| R-work | 0.2155 | (0.2642) |
| R-free | 0.2636 | (0.2927) |
| Number of non-hydrogen | 3919 | |
| atoms | | |

| | |
|---|---|
| macromolecules | 3619 |
| ligands | 23 |
| solvent | 277 |
| Protein residues | 451 |
| RMS (bonds) | 0.009 |
| RMS (angles) | 0.93 |
| Ramachandran favored (%) | 96.41 |
| Ramachandran allowed (%) | 2.91 |
| Ramachandran outliers (%) | 0.67 |
| Rotamer outliers (%) | 4.38 |
| Clashscore | 15.29 |
| Average B-factor | 25.26 |
| macromolecules | 24.93 |
| ligands | 37.19 |

Values in parentheses are for highest-resolution shell.
Data were obtained from a single crystal.

As demonstrated herein, PTM prediction models and growing PTM databases have provided abundant resources for PTM research. However, the shortage of systematic dynamics underlying PTM sites limits the understanding of PTM functions and presents challenges for drug design. In drug design targeting the kinase family, allosteric inhibitors display a greater variety of binding modes and mechanisms than orthosteric inhibitors, with higher selectivity and less acquired resistance. However, the identification of allosteric kinase inhibitors is far from routine and has often been serendipitous. Allostery can be expressed by small or large conformational (enthalpic) and/or dynamics (entropic) changes. Even though allostery can take place in single molecules through covalent PTMs, its consequences propagate through their interactions, which may eventually span the cell. Confirming allosteric mechanisms of action is therefore prone to complications. In the present study, a methodology is proposed of how to systematically investigate the sequence, topological, and dynamics features underlying the biophysical principle of PTMs, as well as how to guide the drug design for the kinase family.

In the relationship between sequence variability and structural dynamics, the orthosteric residues comply with general rules, in which the most conserved residues have the highest stability, being a prerequisite for their precise function. However, the situation is different in PTM sites; the PTM substrates possess certain conservation in evolutionary processes, but they harbor the largest fluctuations, facilitating adaptability of the structure to accommodate spatial changes induced by PTMs. Notably, the PTM sites possess high evolutionary coupling and dynamics coupling with orthosteric residues, at both the residue and pocket levels. The high values for responses upon perturbing orthosteric residues were also observed for PTM sites, further emphasizing their high potential as allosteric pockets. The comprehensive characterization of amino acid dynamics not only has revealed molecular effect and functional landscape of PTMs in the kinase family, but also suggested that dynamics features, beyond widely applied sequence- and structure-based features, could enhance the ability of PTM sites and pockets predictions. Similar ideas have been proposed for the pathogenicity of missense variant prediction^{21a}, and Active and Regulatory site Prediction (AR-Pred)³⁰ by taking advantage of efficient evaluation of structural dynamics by ENMs. Herein, the utility of machine learning models is introduced and demonstrated for classifying PTM sites, in which dynamics features have clear biophysics meaning.

Based on these findings, a “dynamics-allostery-drug design” paradigm is proposed for the PTM-inspired drug design. By focusing on c-Src as a case study, a PTM pocket has been detected, which obeyed this paradigm and highlighted its dynamics and allosteric importance. The subsequent identification of covalent inhibitor DC-Srci-6668 targeting this PTM pocket, adjacent to the ATP-binding pocket, confirmed the feasibility of PTM inspired drug design in the kinase. This inhibitor successfully targeted the PTM pocket of c-Src and precisely regulated the phosphorylation of c-Src to inhibit kinase activation. This methodology should accelerate the design of dual inhibitors that simultaneously interfere with the ATP-binding pocket and PTM sites, thus overcoming the drug resistance problem. Furthermore, the distant PTM pockets, which can regulate the kinase active center through allosteric regulation, will better enlarge the target space for drug design.

In the era of omics, a systematic mapping of PTMs and interactomes into protein structures, deepens the understanding of the links between genotypes and phenotypes and the perturbations that are associated with the onset and progression of various diseases. Inspired by the success of machine learning models in PTM type and site predictions, the introduction of dynamics and allosteric features herein would greatly accelerate the prediction of PTM functions, and the identification of allosteric pockets induced by PTMs. It is therefore foreseeable that in the period of “Big Data”, from the PTMomics and Interactomics, to the “Artificial Intelligence” based on the deep learning models, more PTMs will be identified as the novel biomarkers in the early disease diagnosis and more disease relevant PTMs will be accurately predicted. With an increased understanding of PTMs, such as PTMs involved in diseases and PTM crosstalk, the extended range of biological targets with PTM isoforms would largely enrich personalized treatment opportunities through precision medicine.

### Experimental

### Dataset and Processing

The PTM information for the kinase family was obtained from the PSP database (http://www.phosphosite.org/)^{3c}. The “Regulatory sites” dataset from PSP provided a selection of PTM sites from low throughput experiments that regulated molecular functions, downstream cellular processes and protein-protein interactions. The “Disease-associated sites” data provided PTMs correlated with specific disease states from the literature. The PTMs from the “Regulatory sites”, “Disease-associated sites” and “PTMVar dataset” were defined as regulatory PTM sites. The initial dataset, only considering the monomeric kinase domain in complex with orthosteric inhibitors, included 84 kinase proteins, incorporating 836 PTM sites. The detailed information was listed in Table 2 (FIG. 14).

### PCA of Experimental Structural Ensemble

Crystal structures of the representative kinases were collected and listed in Table 3. The experimental structural data was analyzed using principal component analysis (PCA) by decomposing the covariance matrix C for a dataset as C=Σᵢ₌₁^{3N}σᵢp⁽ⁱ⁾p^{(i)T}, in which p⁽ⁱ⁾ and σᵢ are the ith eigenvalue and eigenvector of C, respectively. The fractional contribution of p⁽ⁱ⁾ to the structural variance is given by fᵢ=σᵢ/Σⱼσⱼ where the summation is performed over all components. The square displacement of the k^{th} residue along p⁽¹⁾ and p⁽²⁾ (also named PC1 and PC2) is (ΔRₖ)²|_{1≤i≤2}=tr{[Σᵢ₌₁²σᵢp⁽ⁱ⁾p^{(i)T}]ₖₖ} in which the subscript kk denotes the k^{th} diagonal element of 3N×3N matrix enclosed in square brackets⁴¹.

TABLE 3
Structural ensembles of the representative kinases, including
CDK2 structures, AKT1 structures, c-Src structures, PAK1
structures, CHK2 structures and RIPK1 structures

| | | | | | | | | | |
|---|---|---|---|---|---|---|---|---|---|
| CDK2 | 1AQ1 | 1B38 | 1B39 | 1CKP | 1DI8 | 1DM2 | 1E1V | 1E1X | 1E9H |
| | 1F5Q | 1FIN | IFVT | 1GII | 1GIJ | 1GZ8 | 1H00 | 1H01 | 1H07 |
| | 1H08 | 1H0V | 1H0W | 1H1P | 1H1Q | 1H1R | 1H1S | 1H24 | 1H25 |
| | 1H26 | 1H27 | 1HCK | 1HCL | 1JSU | 1JSV | 1JVP | 1KE5 | 1KE6 |
| | 1KE7 | 1KE8 | 1KE9 | 1 OI9 | 1OIQ | 1OIR | 1OIT | 1OIU | 1OIY |
| | 1OKV | 1OKW | 1P2A | 1P5E | 1PKD | 1PW2 | 1PXI | 1PXJ | 1PXL |
| | 1PXN | 1PXO | 1PXP | 1PYE | 1QMZ | 1R78 | 1URW | 1V1K | 1VYW |
| | 1VYZ | 1W0X | 1W8C | 1W98 | 1WCC | 1Y8Y | 1Y91 | 1YKR | 2A0C |
| | 2A4L | 2B52 | 2B53 | 2B54 | 2B55 | 2BHE | 2BPM | 2BTR | 2BTS |
| | 2C5N | 2C5O | 2C5Y | 2C68 | 2C69 | 2C6I | 2C6K | 2C6L | 2C6M |
| | 2C6O | 2CCH | 2CJM | 2CLX | 2DS1 | 2DUV | 2EXM | 2FVD | 2G9X |
| | 2HIC | 2IW6 | 2IW8 | 2IW9 | 2J9M | 2R3F | 2R3G | 2R3H | 2R3I |
| | 2R3J | 2R3K | 2R3L | 2R3M | 2R3N | 2R3O | 2R3P | 2R3Q | 2R3R |
| | 2R64 | 2UUE | 2UZE | 2UZL | 2UZN | 2UZO | 2V0D | 2VTA | 2VTH |
| | 2VTI | 2VTJ | 2VTL | 2VTM | 2VTN | 2VTO | 2VTP | 2VTQ | 2VTR |
| | 2VTS | 2VTT | 2VU3 | 2VV9 | 2W05 | 2W06 | 2W17 | 2W1H | 2WEV |
| | 2WIH | 2XMY | 2XNB | 3BHT | 3BHU | 3BHV | 3DDQ | 3EZR | 3EZV |
| | 3F5X | 3FZ1 | 3IG7 | 3IGG | 3LE6 | 3LFN | 3LFQ | 3LFS | 3MY5 |
| | 3NS9 | 3PJ8 | 3PXF | 3PXQ | 3PXR | 3PXY | 3PXZ | 3PY0 | 3PY1 |
| | 3QHR | 3QHW | 3QL8 | 3QQF | 3QQG | 3QQH | 3QQJ | 3QQK | 3QQL |
| | 3QRT | 3QRU | 3QTQ | 3QTR | 3QTS | 3QTU | 3QTW | 3QTX | 3QTZ |
| | 3QU0 | 3QWJ | 3QWK | 3QX2 | 3QX4 | 3QXO | 3QXP | 3QZF | 3QZG |
| | 3QZH | 3QZI | 3R1Q | 3R1S | 3R1Y | 3R28 | 3R6X | 3R71 | 3R73 |
| | 3R7E | 3R7I | 3R7U | 3R7V | 3R7Y | 3R83 | 3R8L | 3R8M | 3R8P |
| | 3R8U | 3R8V | 3R8Z | 3R9D | 3R9H | 3R9N | 3R9O | 3RAH | 3RAI |
| | 3RAK | 3RAL | 3RJC | 3RK5 | 3RK7 | 3RK9 | 3RKB | 3RM6 | 3RM7 |
| | 3RMF | 3RNI | 3ROY | 3RPO | 3RPR | 3RPV | 3RPY | 3RZB | 3S00 |
| | 3S0O | 3S1H | 3S2P | 3SQQ | 3SW4 | 3SW7 | 3TI1 | 3TIY | 3TIZ |
| | 3TNW | 3ULI | 3UNJ | 3UNK | 3WBL | 4ACM | 4BCK | 4BCM | 4BCN |
| | 4BCO | 4BCP | 4BCQ | 4BGH | 4BZD | 4CFN | 4CFU | 4CFV | 4CFW |
| | 4D1X | 4D1Z | 4EK3 | 4EK4 | 4EK5 | 4EK6 | 4EK8 | 4EOI | 4EOJ |
| | 4EOL | 4EOM | 4EON | 4EOO | 4EOP | 4EOQ | 4EOR | 4ERW | 4EZ3 |
| | 4EZ7 | 4FKG | 4FKI | 4FKJ | 4FKL | 4FKO | 4FKP | 4FKQ | 4FKR |
| | 4FKS | 4FKT | 4FKU | 4FKV | 4FKW | 4GCJ | 4I3Z | 4II5 | 4KD1 |
| | 4LYN | 4NJ3 | 4RJ3 | 5A14 | 5AND | 5ANE | 5ANG | 5ANI | 5ANJ |
| | 5ANK | 5ANO | 5CYI | 5D1J | 5FP5 | 5FP6 | 5IEV | 5IEX | 5IEY |
| | 5JQ5 | 5JQ8 | 5K4J | 5LMK | 5MHQ | 5OO0 | 5OO1 | 5OO3 | 5OSJ |
| | 5OSM | 6ATH | 6GUC | 6GUE | 6GUH | 6GUK | 6GVA | 6INL | 6JGM |
| | 6OQI | 6Q3B | 6Q3C | 6Q3F | 6Q48 | 6Q49 | 6Q4A | 6Q4B | 6Q4C |
| | 6Q4D | 6Q4E | 6Q4F | 6Q4H | 6Q4I | 6Q4J | 6Q4K | 6RIJ | 6YL1 |
| | 6YL6 | 6YLK | | | | | | | |
| AKT1 | 3CQU | 3CQW | 3MV5 | 3MVH | 3O96 | 3OCB | 3OW4 | 3QKK | 3QKL |
| | 3QKM | 4EJN | 4EKK | 4EKL | 4GV1 | 5KCV | 6BUU | 6CCY | 6HHF |
| | 6HHG | 6HHH | 6HHI | 6HHJ | 6NPZ | 6S9W | 6S9X | | |
| c-Src | 1KSW | 1Y57 | 1YI6 | 1YOJ | 1YOL | 1YOM | 2BDF | 2BDJ | 2H8H |
| | 2SRC | 4K11 | 4MXO | 4MXX | 4MXY | 4MXZ | 6ATE | 6E6E | |
| PAK1 | 1F3M | 1YHV | 1YHW | 2HY8 | 3FXZ | 3FY0 | 3Q4Z | 3Q52 | 3Q53 |
| | 4DAW | 4EQC | 4O0R | 4O0T | 4P90 | 4ZJI | 4ZJJ | 4ZLO | 4ZY4 |
| | 4ZY5 | 4ZY6 | 5DEW | 5DEY | 5DFP | 5IME | 5KBQ | 5KBR | 6B16 |
| CHK2 | 2CN5 | 2CN8 | 2W0J | 2W7X | 2WTC | 2WTD | 2WTI | 2WTJ | 2XBJ |
| | 2XK9 | 2XM8 | 2XM9 | 2YCF | 2YCQ | 2YCR | 2YCS | 2YIQ | 2YIR |
| | 2YIT | 4A9R | 4A9S | 4A9T | 4A9U | 4BDA | 4BDB | 4BDC | 4BDD |
| | 4BDE | 4BDF | 4BDG | 4BDH | 4BDI | 4BDJ | 4BDK | | |
| RIPK1 | 4ITH | 4ITI | 4ITJ | 4NEU | 5HX6 | 5TX5 | 6C3E | 6C4D | 6HHO |
| | 6NW2 | 6NYH | 6OCQ | 6R5F | 6RLN | | | | |

CDK2: The reference structure is 4I3Z. Residues 1-296 were included in the analysis, corresponding to 99.3% of CDK2 sequence. These structures were resolved at 1.0 Å resolution or higher.
AKT1: The reference structure is 4GV1. Residues 143-451, 457-477 were included in the analysis, corresponding to 68.8% of AKT1 sequence. These structures were resolved at 1.49 Å resolution or higher.
c-Src: The reference structure is 1Y16. Residues 258-533 were included in the analysis, corresponding to 51.5% of SRC sequence. These structures were resolved at 1.5 Å resolution or higher.
PAK1: The reference structure is 4DAW. Residues 249-542 were included in the analysis, corresponding to 53.9% of PAK1 sequence. These structures were resolved at 1.64 Å resolution or higher.
CHK2: The reference structure is 4BDK. Residues 211-227, 232-253, 267-513 were included in the analysis, corresponding to 52.7% of CHK2 sequence. These structures were resolved at 1.77 Å resolution or higher.
RIPK1: The reference structure is 6RLN. Residues 8-19, 30-176, 188-294 were included in the analysis, corresponding to 39.6% of RIPK1 sequence. These structures were resolved at 1.80 Å resolution or higher.

### Sequence Information

Protein family sequences from the NCBI were searched and multiple sequence alignments (MSA) were obtained by Clustal Omega⁴². Shannon entropy was calculated for each position in the MSA to assess the conservation of residues using Evol^{28,43}, a python module in ProDy. To evaluate the coevolution for residue pairs, the Direct Coupling (DI) analysis matrix, mutual information (MI) matrix were calculated, observed minus expected squared (OMES) covariance matrix, and the statistical coupling analysis (SCA) matrix between the positions of the MSA.

### Features of the Protein Structure

Calculations were performed for solvent accessibility using DSSP with default parameters. The DSSP program was used to assign the secondary structures 44. DSSP assigns a single letter code (H, S, G, T, E, B, and I, -) to each residue corresponding to the secondary structural type.

Fpocket was used to predict cavities or pockets from atom positions in protein structures and identified the residues that were located in pockets⁴⁵. Fpocket uses alpha spheres and Voronoi tessellations to identify pockets in a protein. It considers a residue to be part of a pocket if any of the residue atoms are at a distance equal to the radius of an alpha sphere in the pocket.

### Protein Structure Network

Bio3D (R package) was used to model the protein structure networks (PSNs)⁴⁶. The normal mode input was first subjected to the correlation analysis. Each protein structure was rendered as a coarse-grained network whose nodes are residues represented by their Cu atoms. These residues were connected by weighted edges proportional to the extent of dynamic correlations. Subsequently, the node betweenness, closeness, degree, clustering coefficient, and average shortest path length were calculated.

### Elastic Network Model

Each protein was modeled as a coarse-grained Elastic Network Models (ENMs) by representing its N residues by their respective Ca atoms and connecting all pairs of residues with harmonic springs. Herein, the two most commonly used ENMs, the Anisotropic Network Model (ANM) and Gaussian Network Model (GNM), were adapted to elucidate the equilibrium dynamics of protein structures. Knowledge of the distribution of inter-residue contacts in the native structure allowed us to construct the Kirchhoff (GNM) and Hessian (ANM) matrices, upon which eigenvalue decomposition yielded information on the collective modes. Both GNM and ANM analyses were performed by using the ProDy package 4. Subsequently calculated were the mean-square fluctuations (MSF) and cross correlation values in both ANM and GNM models.

### Perturbation Response Scanning

Perturbation response scanning (PRS) allows for a quantitative assessment of the influence/sensitivity of each residue with respect to each other⁴⁸. The results are described by N×N heat maps (for a protein of N residues). The row and column averages provide two dynamics features to describe the allosteric potential of residues, while the residues (sites) with largest values based on this dual profiling usually populate two mutually exclusive sets of residues act as sensors or effectors.

### Features Summary

For each protein, features at the residue level were calculated and each residue was represented as a vector of different features. Based on how they were calculated and what aspect they represented, these features were broadly grouped into three categories: (a) SEQ features from protein sequence evolution, (b) STR features describing structure geometry and network topology, and (c) DYN features for protein dynamics and perturbation responses. All features were presented using the violin plots. The violin plot was used to display the distribution status and probability density of multiple sets of data. In analyzing the difference underlying PTM residues, orthosteric residues, and others, the Wilcox rank sum test was used and P values are represented in the plot.

### Machine Learning Models and Performance Evaluation

The supervised classification of residues into PTM sites, orthosteric sites and non-functional sites, based on the features described in the previous section, were conducted through a Random Forest (RF) classifier and Fully Connected Neural Network (FCNN) methods.

The RF algorithm builds an ensemble of decision trees fitted to the training data, and assigns a label based on the consensus from all trees. Used in the present study was implementation of the RF algorithm included in the open source Python library Scikit-learn. The main parameters, namely the number of trees and the maximum number of features used for fitting, were optimized through cross-validation. Because most data sets were strongly imbalanced, with generally a much larger number of non-functional sites than PTM sites and orthosteric sites (see Table 2, FIG. 14), the “balanced” option in Scikit-learn was used to automatically assign weights to classes that were inversely proportional to their frequencies in the training set. FCNN is a feed-forward artificial neural network, which consists of three-layer types: one input layer, one or more hidden layers, and one output layer. The input vector is fed into the neural network architecture, where each layer serves as the input for the next layer by weighted connections. The detailed description of the models is provided in Supporting Information.

The model performance was evaluated using receiver operating characteristic (ROC) area under the curve (AUC) for different values of true positive and false positive rates. The area under the ROC is denoted as AUC.

### Compound Library

The in-house compound library was derived from the covalent inhibitors that have been reported and solved the complex crystal structure with target biomacromolecule. The compounds sorted and collected from the PDB were first manually selected based on experience, and then structural similarity search was performed in the open-source compound databases from ChemDiv (https://www.chemdiv.com/) and SPECS (https://www.specs.net/) to improve structural diversity. Finally, 720 compounds with covalent warheads were obtained and purchased from commercial supplier TargetMol (USA). All compounds were dissolved in DMSO before application.

### Covalent Docking

In the covalent docking, each compound was prepared with the LigPrep module and c-Src was prepared with the Protein Preparation Wizard module in the Schrodinger software package⁴⁹. In the Covalent docking module, C280 was selected as the reactive residue in c-Src kinase, and nucleophilic substitution was selected as the reaction type. Scoring function was used to characterize the fitness between the docked compounds and surrounding residues within the binding pocket. Electrostatic and Van der Waals energy were the main provisions of the scoring functions. For the calculation of electrostatic energy, the atomic charges for the protein were calculated with Tripos force field parameters. For the calculation of Van der Waals energy, the Lennard-Jones (6-12) potential was used. Finally, the program gave an output of the best score for each compound, as well as the corresponding conformations.

### Protein Expression and Purification

The recombinant flag-tag human c-Src protein (86-536) with TEV restriction site was cloned into pFBDM vector and expressed in Sf9 insect cells by Bac-to-Bac system (Invitrogen). The cells were infected with baculovirus at 27° C. for 48-72 hours before collection. Cells were lysed in buffer containing 20 mM Hepes pH=7.4, 150 mM NaCl, 1 mM DTT, 1× protease inhibitor cocktail (Roche) and 1 mM PMSF. Cell lysate supernatant was loaded to column packed with anti-Flag G1 affinity resin (GenScript), washed by lysis buffer, and finally eluted with 0.2 mg/mL flag peptide (GenScript). TEV enzyme was added to digest falg-tag overnight at 4° C. The collection sample was concentrated and loaded onto Superdex™ 200 Increase 10/300 GL column (GE Healthcare) for further purification and exchange the buffer to 20 mM Hepes pH=7.4, 150 mM NaCl, 1 mM DTT.

Human c-Src kinase domain (Src-k, including WT and C280S, 254-536) with a TEV protease cleavable N-terminal 6×-His tag was cloned into pET28a vector and co-expressed with full length YopH phosphatase cloned to pCDFDuet-1 vector in Escherichia coli BL21 (DE3) cells. Cells were cultured in LB medium at 37° C. and induced with 0.4 mM IPTG at 18° C. for 16 hours. Proteins were purified using HisTrap FF column (GE Healthcare) in buffer containing 50 mM Hepes pH=8.0, 500 mM NaCl, 5% glycerol and imidazole (25 mM for loading, 150 mM for elution). After TEV enzyme cleavage, proteins were loaded to Hitrap Q FF (GE Healthcare) in buffer containing 20 mM Hepes pH=8.0, 5% glycerol, 1 mM DTT (QA) and eluted with a linear gradient of 10-30% buffer QB (buffer QA plus 1M NaCl). Then proteins were further purified by Superdex75™ 10/300 GL column (GE Healthcare) and change the buffer to 20 mM Hepes pH=7.4, 150 mM NaCl, 1 mM DTT.

### Homogeneous Time-Resolved Fluorescence Assay

The ability of compounds to inhibit the phosphorylation of a peptide substrate by c-Src kinase was evaluated by homogeneous time-resolved fluorescence (HTRF) using KinEASE-TK kit (Cisbio, Bedford, MA, USA). It is a generic method for measuring tyrosine kinase activities by detecting the phosphorylation level of substrate (http://www.cisbio.com/kinases). First, 100 nM c-Src protein was incubated with compounds at the set concentration for 1 hour at room temperature. Next, equal volume of mix with 20 μM ATP, 10 mM MgCl₂ and 100 nM biotinylated TK-substrate peptides were added to initiate enzymatic reaction. The reaction was proceeded at 37° C. for 1 hour. Then, Eu³⁺-cryptate labeled phosphorylation antibody and streptavidin-XL665 was added to stop the reaction and start the detection step. The detection step was proceeded at room temperature for 1 hour. Finally, fluorescence was measured at 615 nm and 665 nm using EnVision reader (PerkinElmer). The results were calculated as follows: ratio=OD665/OD615 and the IC₅₀ values were analyzed in Graphpad Prism 8.0.

### Mass Spectrometry Analysis

c-Src protein was diluted to approximately 20 μM in final buffer and incubated with compound at a final concentration of 100 μM or same volume of DMSO at 4° C. for 8 hours. Then, the protein samples were diluted into aqueous solution containing 0.1% formic acid (about 1 mg/mL), and 2 μg of the target protein samples were took for LC/MS analysis. Intact protein high-resolution mass spectrometry was performed using Ultimate 3000 LC liquid chromatograph and LTQ Orbitrap mass spectrometer equipped with HESI ion source (Thermo Fisher, CA). BioPharma Finder software (version 2.0, Thermal Fischer, California) was used to process the raw LC-MS data, and the ReSpect™ deconvolution algorithm was used to obtain the intact protein masses.

### Protein Thermal Shift

Protein thermal shift (PTS) assays were performed on a QuantStudio™ 6 Flex Real-time PCR system (Applied Biosystems). 5M Src-k protein, 5×SYPRO® orange (Molecular Probes) and different concentrations of compounds were mixed in 20 μL final buffer and added to 96-well plates (DN Biotech). According to the standard protocol, the reaction system was heated from 25° C. to 95° C. within 25 minutes, and the fluorescence signal was monitored in real time. Protein Themal Shift™ Software Version 1.2 (Life Technologies) was used to determine the Tm value and Graphpad Prism 8.0 was used to draw the curves. The Y419-phosphorylated protein used was obtained by incubating Src-k protein with 5 mM MgCl2 and 10 mM ATP overnight, and then desalting it into final buffer.

### Amplified Luminescent Proximity Homogeneous Assay

The ability of compounds to inhibit the auto-phosphorylation of c-Src Y419 was determined by Amplified Luminescent Proximity Homogeneous Assay (ALPHA). ALPHA assay was carried out in assay buffer containing 20 mM Hepes, pH=7.4, 0.1% Triton X-100, 1 mM DTT, 0.1% bovine serum albumin (w/v)). First, 10 nM His-tag Src-k protein was incubated with compounds for 1 hour at room temperature. Then equal volume of mix with 20 μM ATP, 10 mM MgCl2 was added, and the reaction was proceeded at 37° C. for 30 minutes. Subsequently, ALPHA anti-His donor beads, protein A coated acceptor beads (PerkinElmer) and c-Src Y419-phophorylation antibody (Cell Signaling Technology) were added to the reaction system and incubated it at room temperature for 1 hour. Finally, the signals were measured in ALPHA protocol using EnVision reader and the IC₅₀ values analyzed in Graphpad Prism 8.0.

### Determination of Complex Crystal Structure

The inactive form of c-Src was increased by incubating with Csk as previous reported⁵⁰. Then it was treated with DC-Srci-6668 for 8 hours at a molar ratio of 1:10, and loaded onto Superdex™ 200 Increase 10/300 GL column to remove unstable protein polymers and excess compounds. Crystals were obtained at 16° C. for 3-5 days using the hanging drop vapor diffusion method by mixing equal volume of protein solution (concentrated to 10 mg/mL) and reservoir solution (20% PEG3350, 200 mM tri-Lithium citrate). Diffraction data were collected at the BL19U1 beamlines at Shanghai Synchrotron Radiation Facility. Data were processed and integrated using the HKL3000. The initial structure was solved using the molecular replacement module of Phenix with the template of human c-Src (PDB code: 2SRC). After that, rounds of refinement were performed using the Phenix, and Coot was adopted to correct the mismatched electron density during the whole refinement period.

### Features Used in the Machine Learning Models

Features for classification of PTM sites were used, composed of sequence (SEQ), structural (STR) and dynamics (DYN) based features. SEQ features were evaluated using the multi-sequence alignment for the sequence corresponding to the kinase proteins, calculated with the Evol package in ProDy Python API. STR features were evaluated by solvent accessible areas and protein structure networks, calculated with DSSP and Bio3D. DYN features were based on elastic network models (ENMs), calculated with the ProDy Python API. Here, a brief description is provided for each of them.

### Sequence-Based (SEQ) Features

Conservation and co-evolution are based on the analysis of multiple sequence alignment (MSA) built for the examined protein. Such conservation properties are extremely informative, such as in missense variants prediction.
a. Conservation: For any random variable X, its Shannon entropy is defined as following:

$$H ( X ) = - {\sum }_{i} P ( {x}_{i} ) {log}_{2} P ( {x}_{i} ) \qquad ( 1 )$$

Where i is the total number of all sequences, P(xᵢ) denotes the probability function of X.
b. Mutual information matrix (MI): The mutual information between two discreet random variables X, Y jointly distributed according to p(x, y) is given by

$$I ( X ; Y ) = {\sum }_{x , y} p ( x , y ) log \frac{p ( x , y )}{p ( x ) p ( y )} \qquad ( 2 )$$

c. Observed minus expected squared (OMES) covariance matrix: OMES is defined as:

$$OMES ( i , j ) = sum ( \frac{{( N\_OBS - N\_EX )}^{2}}{N\_EX} ) = N * sum ( \frac{{( {f}_{i} j - {f}_{i} * {f}_{j} )}^{2}}{{f}_{i} * {f}_{j}} ) \qquad ( 3 )$$

d. The statistical coupling analysis (SCA) matrix: SCA is defined as:

$${C}_{ij} = \vert \frac{1}{N} {\sum }_{k} {x}_{ki} {x}_{k j} - \frac{1}{{N}^{2}} {\sum }_{k , l} {x}_{k i} {x}_{l j} \vert \qquad ( 4 )$$

DI_bind, MI_bind, OMES_bind and SCA_bind were calculated by retaining the DI, MI, OMES and SCA matrices all rows and the columns where binding sites were located, and then averaging the row values of each residue.

### Structural-Base (STR) Features

### Solvent Accessibility

The solvent accessible surface area is the area of the surface swept out by the center of a probe sphere rolling over a molecule (atoms are spheres of varying radii). The solvent accessible surface is just the boundary of the union of atom balls that have their radius increased by the probe radius (typically 1.4 Angstroms). So the accessible area is the surface area of a union of balls.

### Network Centralities

For protein structure network (PSN) models, the following node centralities were calculated.
a. Betweenness. Node betweenness is defined as the number of current nodes in the shortest path of the network. The betweenness bᵢ of a node i is defined as:

$${b}_{i} = {\sum }_{j , k ∈ N , j \neq k} \frac{{n}_{jk} ( i )}{{n}_{j k}} \qquad ( 5 )$$

where nⱼₖ is the number of shortest paths connecting j and k, while nⱼₖ(i) is the number of shortest paths connecting j and k and passing through i.
b. Closeness. Node closeness characterizes the local measure, reflecting the association ability of the node itself, and does not consider the control problem for other nodes. Closeness indicates how closely the current node is connected to all other nodes, which is defined by the inverse of the average length of the shortest paths to/from all the other vertices in the graph:

$${c}_{i} = 1 / {\sum }_{i \neq j} d ( i , j ) \qquad ( 6 )$$

where d(i, j) is shortest path length.
c. Clustering Coefficient. The clustering coefficient, is sometimes also called the transitivity. There are several characterizations of clustering coefficient to weighted graph, here a local vertex-level quantity was used, and its definition is as:

$${C}_{i}^{ω} = \frac{1}{{s}_{i} ( {k}_{i} - 1 )} {\sum }_{j , h} \frac{{ω}_{ij} + {ω}_{i h}}{2} {a}_{i j} {a}_{i h} {a}_{j h} \qquad ( 7 )$$

where sᵢ is the strength of vertex i, and the strength is defined as summing up the edge weights of the adjacent edges for each node. αᵢⱼ are elements of the adjacency matrix A, kᵢ is the node degree, and ωᵢⱼ are the weights.
d. Degree. The degree of a vertex is defined by the number of its adjacent edges incident with the vertex. The degree kᵢ of a node i is defined in terms of the adjacency matrix as:

$${k}_{i} = {\sum }_{j ∈ N} {a}_{ij} \qquad ( 8 )$$

where aᵢⱼ (i, j=1, . . . , N) is from a N×N adjacency matrixA, entry aᵢⱼ is equal to 1 when the link lᵢⱼ exists, and zero otherwise.
e. Shortest path. Since there may be multiple paths from one node v₁ to another node vⱼ in the network, the number of edges traversed by each path may be different, that is, the path length is different. The path with the least number of edges is called the shortest path.

### Dynamics-Base (DYN) Features

a. Square Fluctuations. In both GNM and ANM, the topological and dynamics information are saved in Kirchhoff matrix Γ and Hessian matrix H, respectively. The eigenvalue decomposition of F and H yield N-1 and 3N-6 eigenvalues λₖ and eigenvectors uₖ, corresponding to the frequencies and normal modes. As such, square fluctuations can be defined as:

$$〈 Δ {R}_{i} \cdot Δ {R}_{i} 〉 = 〈 {( Δ {R}_{i} )}^{2} 〉 = ( 3 {k}_{B} T / γ ) {{\sum }_{k} [ {( {u}_{k} ∧ {u}_{k}^{T} )}^{- 1} ]}_{i i} \qquad ( 9 )$$

where γ is the force constant assumed to be uniform for all springs in the network, T is the absolute temperature and k_{B} is the Boltzmann constant, and thus ΔRᵢ is a vector that represents the displacement of the ith residue from its equilibrium position.
b. Cross correlations. Cross-correlations provide information on the relative movements of pairs of residues. The normalized version of this correlation is given by

$${C}_{i j} = \frac{〈 Δ {R}_{i} \cdot Δ {R}_{j} 〉}{{[ 〈 Δ {R}_{i} \cdot Δ {R}_{i} 〉 〈 Δ {R}_{j} \cdot Δ {R}_{j} 〉 ]}^{1 / 2}} \qquad ( 10 )$$

The value of Cᵢⱼ is between −1 and 1. The greater the absolute value of Cᵢⱼ, the higher the correlation between the two residues. Cᵢⱼ can been calculated by both GNM and ANM based on different single eigenvalue and their combinations.
c. Perturbation Response Scanning (PRS). Effectiveness/sensitivity properties are both derived from the PRS analysis, based on an ANM or GNM model. The PRS is defined as:

$$Δ R = {( B K {B}^{T} )}^{- 1} Δ F \qquad ( 11 )$$

where B is direction cosine matrix, K is coefficient matrix and ΔF is the forces necessary to induce a given point-by-point displacement of residues. The generic element mᵢⱼ of PRS matrix represents the impact of a point perturbation at residue i as measured at residue j. Column and row averages of the PRS matrix describe respectively the effectiveness of a residue in transmitting deformation signals to the whole protein and the sensitivity of a residue to such deformations localized at other sites.

### Random Forest (RF) Model and Deep Learning Model

The categories for PTMs, orthosteric residues and other residues (substracting PTMs and orthosteric residues ±5 amino acids window), were modeled by random forest (RF) and Fully Connected Neural Network (FCNN) methods. Both models were fine-tuned using 10-fold cross-validation and validated on independent test dataset. The RF model construction procedure was executed using the Seikit-learn toolkit. All of features were weighted equally in estimator of individual tree and single node of perceptron. RF was an ensemble method by aggregating decision trees, where each tree was grown using bootstrapped samples. After exhaustive searching over the parameter space, the number of trees was 100 and the maximum number of features was the square root of the number of features. The default value was used for the maximum depth, and there was no limit on the depth when building the subtree.

In FCNN models with different feature combinations, the number of neurons in each layer was listed in Table 7. Root mean square prop (RMSProp) was used to update the parameters of FCNN models. Rectified linear unit (ReLU) was used in the hidden layers as the activation function, and sigmoid function was applied in the output layer as activation function.

TABLE 7
The number of neurons in each layer of FCNN models

| Feature | Number of units |
|---|---|
| SEQ + STR + DYN | 64, 64, 3 |
| SEQ | 16, 16, 32, 3 |
| STR | 32, 64, 3 |
| DYN | 32, 3 |

### Performance Evaluation

The accuracy of the classification was evaluated by means of the area under curve (AUC) computed over the receiver operating characteristic (ROC) plot. To assess the performance of each model, Accuracy, Sensitivity, Specificity, Precision and F1 score were calculated to measure the performance of models.

### Prediction Models

In the ROC curve for each category (FIG. 11), it was observed that the prediction ability of both models for orthosteric residues (class 2) ranked the first and for PTM residues (class 1) ranked at the bottom. This indicated that the orthosteric residues shared consistent features, such as high conservation and low flexibility, in contrast to the variable features for PTM residues. Although the RF models displayed better AUC values compared with FCNN models, the confusion matrixes (FIG. 11) suggested that the FCNN models possessed better predication accuracy for the three categories, whereas the RF had least prediction accuracy for PTM residues. The predictive nature of the RF model for PTM residues was less significant and was more random than the FCNN model for PTM residues. However, one must consider that orthosteric binding sites are substantially better known and have been investigated more exhaustively in comparison with PTM sties. Orthosteric residues have long been exploited as popular drug targets by pharmaceutical industries and thus, their identification is supported by a plethora of experimental evidence.

### Associated Content

The Python code for model training and analysis, and data set (used in FIG. 4) are freely available at https://aithub.com/ComputeSuda/PTMKinase. The atomic coordinates and structural factors for the complex crystal structure of c-Src_DC-Srci-6668 have been deposited to the Protein Data Bank, Research Collaboratory for Structure Bioinformatics (RCSB PDB, resb.org) with code 7ELU.

Although the present invention has been described in detail with preferred embodiments, those of ordinary skill in the art should understand that modifications, variations, and equivalent replacements made to the present invention within the scope of the present invention belong to the protection of the present invention.

Applicant's disclosure is described herein in preferred embodiments with reference to the Figures, in which like numbers represent the same or similar elements. Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar language throughout this specification may, but do not necessarily, all refer to the same embodiment.

The described features, structures, or characteristics of Applicant's disclosure may be combined in any suitable manner in one or more embodiments. In the description, herein, numerous specific details are recited to provide a thorough understanding of embodiments of the invention. One skilled in the relevant art will recognize, however, that Applicant's composition and/or method may be practiced without one or more of the specific details, or with other methods, components, materials, and so forth. In other instances, well-known structures, materials, or operations are not shown or described in detail to avoid obscuring aspects of the disclosure.

### Example 2

### Data Set

Protein Data Bank (PDB) files were collected from the PDBbind and the RCSB PDB database. In the PDBbind database, the general set minus the refined set data and the refined set data were both collected. The PDB files downloaded from the RCSB PDB Web site provided information such as structural resolution, method, etc. Then several steps were performed to improve the quality of the protein structural data: (a) filtering repetitive proteins and retaining the crystal structures with the highest resolution from X-ray crystallography (2411 unique protein-ligand complexes obtained); (b) filtering proteins in multiple chains (755 monomeric protein-ligand complexes obtained); (c) filtering proteins with protein sequence less than 50 and PTM sites less than 5. At last, 389 protein structures with ligand binding information were collected.

The PTM information was collected from PhosphoSitePlus and dbPTM databases. Specifically, phosphorylation sites obtained from Disease-associated sites, PTMVar, and Regulatory sites files from PhosphoSitePlus database are considered as FuncPhos sites with different molecular mechanisms and phenotypes, and the others without functional annotations are supposed to be nonfunctional phosphorylation sites. In addition, the phosphorylation information was supplemented from the recent functional phosphorylation research. Through sequence alignment, PTM sites were mapped to 389 protein-ligand complex structures, acquiring 4898 PTM sites and 18,269 ligand binding sites.

### Feature Representation.

For each protein, features were calculated at the residue level and residues were each represented as a vector of different features. On the basis of how they were calculated and what aspect of a protein they represented, these features can be broadly grouped into the following categories: (a) Seq features based on sequence evolution, (b) Str features from protein structure geometry, (c) Dyn features describing protein dynamics, and Allo features describing protein communication. In summary, 65 features were calculated for each residue, leading to a 65-dimensional vector for each residue in proteins. Further examples and details of features are described in Zhu et al., “Leveraging Protein Dynamics to Identify Functional Phosphorylation Sites using Deep Learning Models.” J. Chem. Inf. Model., 2022, which is hereby incorporated by reference in its entirety.

### Model Construction and Performance Evaluation.

Facing the imbalance distribution of the samples in phosphorylation, acetylation, and ubiquination (PAU) sites and functional phosphorylation (FuncPhos) sites, the ratio of positive/negative at 1:1 and 1:N were first resampled, in which 1:N was consistent with the ratio in total samples. FuncPhos sites prediction also involved resampling the ratio of positive/negative at 1:2 and 1:3 for 10 times, generating 10 different data sets at each ratio. Second, for each ratio, each data set was divided into a training set and a test set, the model was built on the training set, and the results were obtained on the test set. Finally, the robustness of the models were evaluated on different ratios by averaging the prediction results on multiple test sets.

For each residue, 65 features led to a 65-dimensional vector representation. Meanwhile, a window of size 13 in combined deep learning (cDL) models was set, in which a site was represented by incorporating six sites from both left and right of the target site. Zero-padding was used to form the 65-dimensional vectors if there were less than six sites in the left or right part of the target residue. Therefore, each residue was ultimately represented by a two-dimensional matrix of shape 13×65, which was used as input of the cDL models, while the input data of FNN and RF was the 65-dimensional vector representation of each site, that is, a vector 1×65.

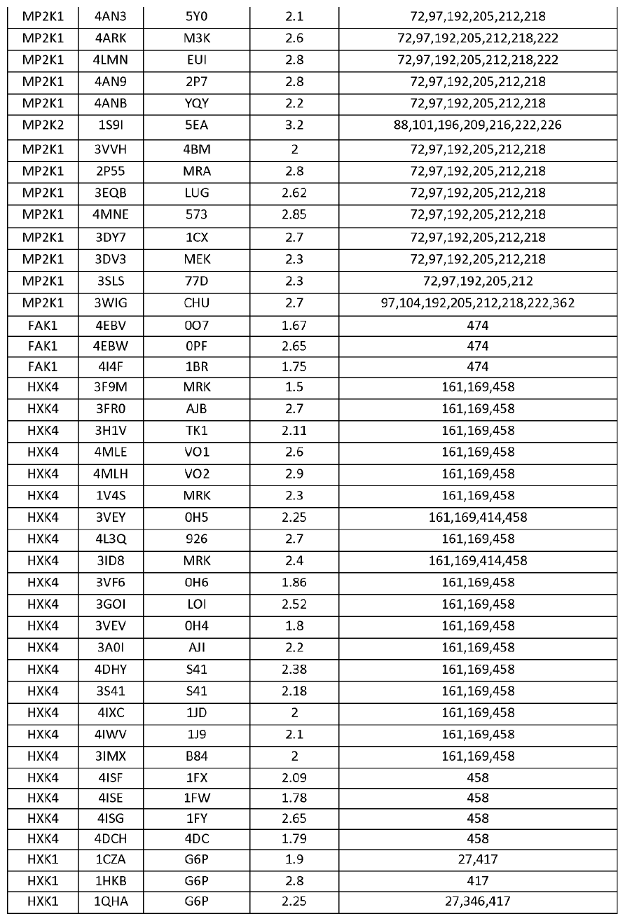

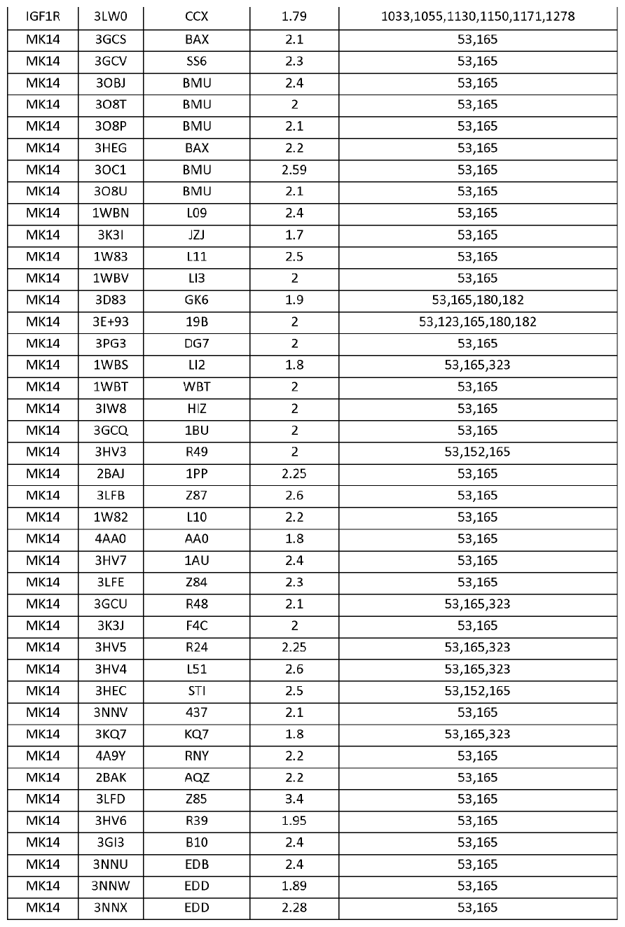

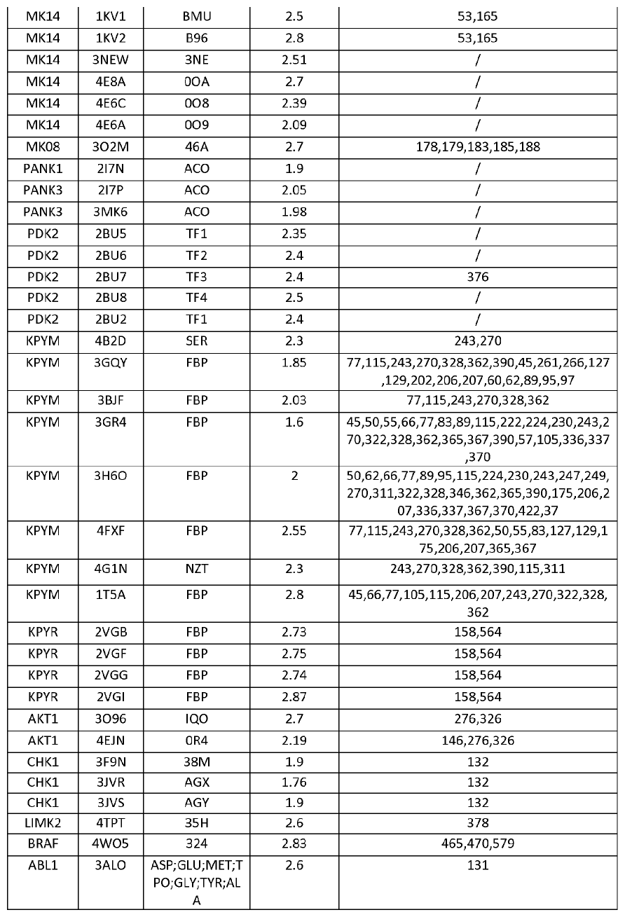

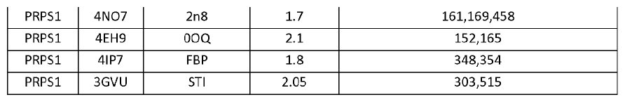

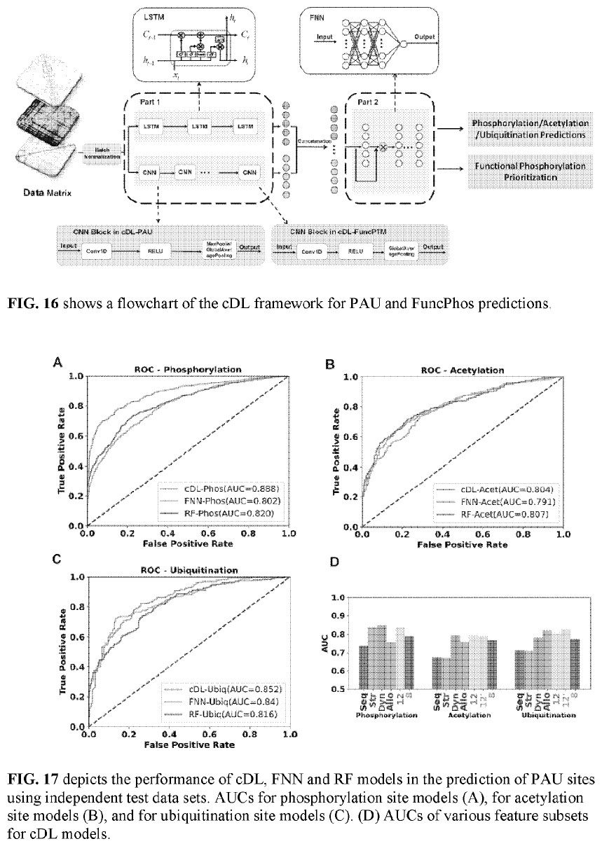

In order to construct the better models, an optimization process was performed by using 10-fold cross-validation on the training sets for each model, seeking for the hyperparameters of the model so that each model could achieve the best prediction result. After analyzing the prediction effects of different parameters on each model, the hyperparameters of the model with the best verification effect were chosen. Referring to the deep learning models, both cDL-PAU and cDL-FuncPhos use similar model structures, including a batch normalization layer and two parts, in which Part 1 consists of three-layer Long Short-Term Memory (LSTM) and four-layer Convolutional Neural Networks (CNN), and Part 2 consists of four layer Fully connected Neural Network (FNN) (FIG. 16). In cDL-PAU, the batch-standardized input data were processed with LSTM modules and CNN modules, and their outputs were concatenated into a vector, which was then fed into an FNN module. The CNN layers were built with one-dimensional (ID) convolution, ReLU, and max polling blocks, whereas the last CNN layer added a global average pooling layer, so that global context information can be fully utilized. In Part 2, the FNN received the concatenate vector from the last layer of the CNN and LSTM to capture the efficient information. The last layer of FNN outputs a probability value that indicates the probability of the site being a phosphorylation, acetylation, or ubiquitination site. In cDL-FuncPhos, the first three CNN layers consisted of convolutional blocks and ReLU. The last CNN layer was added with a global average pooling layer. The output of the CNN and LSTM layers was concatenated into a vector, passed to the first layer of FNN where Softmax was the activation function. The output of the first fully connected layer was a multiplication of the probability with the input to the layer, which was then fed into the subsequent FNNs. Finally, FNN gave the probability of a phosphorylation site being a functional one.

Fully connected Neural Networks (FNNs) were also used in the tasks of predicting PAU, named as FNN-PAU, and FuncPhos sites, named as FNN-FuncPhos. The FNN architecture consisted of the input layer, the hidden layer, and the output layer. Specifically, the input layer is responsible for accepting the input of the data, and the output layer is responsible for outputting the results of neural network predictions, that is, the probability of predicting whether the data belongs to a positive class. The hidden layer in the middle of the FNN is responsible for nonlinear transformation of the input data, extracting the implied information from the data. All the feature vectors were processed with batch normalization before feeding into FNN layers in FNN-PAU. There were four hidden layers in FNNPAU with ReLU as the activation function. The output layer used the sigmoid activation function to predict the probability. This neural network structure was held the same for FNNFuncPhos.

The Random Forest (RF) model has been widely recognized as a powerful tree-based classification algorithm in machine learning. For the classification problem, RF ends up with the most voted class among all the decision trees as the final classification result of RF according to the majority principle. For each task in this study, Scikit-Learn 0.24.2 was used to build the RF models, and 600 trees were constructed for the PAU predictions. The maximum number of features in RF was set to the square of the features. The minimum number of samples required for internal node repartitioning was set to 2, and the minimum number of samples for leaf nodes was set to 1. In the predictions of PAU, the RF for each task consisted of 600 trees. The maximum depths of RF models were assigned 24, 23, and 17 for the predictions of phosphorylation, acetylation, and ubiquitination, respectively. The RF used in FuncPhos was also composed of 600 trees with a maximum depth of 16. In both tasks, default values from Scikit-Lean were used for the maximum number of features, the minimum number of samples required for repartitioning internal nodes, and the minimum number of samples of leaf nodes, respectively. To assess the performance of each model, accuracy, precision, sensitivity, specificity, false positive rate (FPR), false negative rate (FNR), F1 score, and Matthews correlation coefficient (MCC) were computed to quantify the model performance. The area under the curve (AUC) of receiver operating characteristic (ROC) was also calculated. All the evaluations were performed on the independent test data sets.

### Performance Assessment of PAU Prediction Models.

On the basis of the systematic feature engineering procedures, cDL models, FNN models, and RF models were constructed for PAU site predictions. Hyperparameters optimization was achieved with grid search, and model performance evaluation was done with 10-fold cross-validation. In cDL models, a two-dimensional matrix of shape 13×65 for each residue with batch normalization was used as the input of part 1, which consisted of CNN and LSTM as build modules. As for part 2, FNN is the most used neural network for the output of the final prediction result. Whereas in FNN and RF models, the input data was a 65-dimensional vector representation of each residue, that is, a vector 1×65. The detailed parameters of cDL models and FNN models for PAU sites are identical.

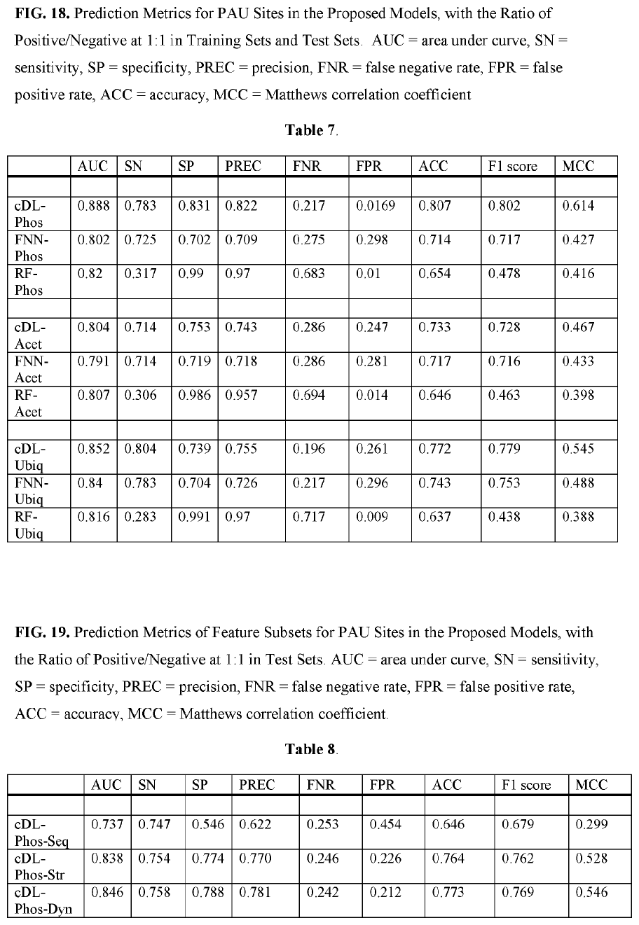

On the basis of a head-to-head comparison in FIGS. 17A-C, the performance of cDL models achieves efficient improvements over FNN and RF models in independent test data sets, with the AUC values ranging from 0.804 for acetylation sites to 0.888 for phosphorylation sites (FIG. 18, Table 7). Specifically, cDL models achieve higher scores in AUC, sensitivity, accuracy, F1 score, and MCC than FNN and RF models. Although the RF models achieve the highest specificity and precision, they also possess the lowest sensitivity and the highest FNR, indicating big shortcomings for RF models to accurately identify the positive samples. The consistent results for the cDL, FNN, and RF models were also observed with the ratio of positive/negative at 1:N in the training sets and test sets. There probably are two reasons for the advantage of cDL models. The first is due to the representation of the data entered into cDL, in which a (13, 65) two-dimensional matrix possesses more information than a 65-dimensional vector used in FNN and RF models. The second is due to the differences in the model structure and their applicable scenario. Compared with FNN and RF, CNN and LSTM modules in cDL are more powerful in extracting efficient feature information, especially for the data in a matrix form.

To explore the molecular features for PAU prediction, the SFS algorithm was adopted to produce the optimal feature subsets based on the four categories. In terms of the prediction tasks (FIG. 19, Table 8), the optimal subset of the Dyn and Allo features generally display advantageous prediction results, especially for the fewer Allo features with higher AUC values. In phosphorylation and acetylation prediction, the optimal Dyn features achieve the highest AUC values of 0.846 and 0.793 (FIG. 19, Table 8), whereas the optimal Allo features achieve the highest AUC value of 0.820 in ubiquitination prediction (FIG. 19, Table 8). In detail, the optimal Allo features emphasized the importance of strong response underlying PAUs and the higher communication efficiency between PAUs and other residues. Concerning Dyn features, the higher eigenvectors for PAUs and higher coupling correlation between PAUs with other residues were captured in a variety of frequencies of ANM modes. For Seq features, the high conservation and high coevolution underlying PAUs were captured. In contrast, the lowest AUC values for acetylation and ubiquitination with an Str subset could be attributed to the not insufficient significant differences in the topological features.

To further optimize the feature combinations, cDL models were generated with 12 features based on the SFS results. As Str features are not good from SFS, the Str features were selected based on the FC and P values from the comparison between PAUs and non-PAU residues. As listed in FIG. 19, Table 8, cDL-Phos-12 model achieves the highest AUC value of 0.841, demonstrating the rationality of feature combinations. As the 12 features for cDL-Acet-12 and cDL-Ubiq-12 achieve AUC values of 0.793 and 0.807, another 12 feature combinations were tried (FIG. 19, Table 8), and the minor changes suggest the robust predictive ability for these features. The further combination of eight features (FIG. 19, Table 8) resulted in decreased AUC values from 0.767 to 0.787. Hence the cDL models with the combination of 12 features with AUC values from 0.793 to 0.841 would be the better choice in PAU predictions. The shared optimal feature subsets underlying PAU sites suggest the intrinsic molecular features in characterizing PTMs.

Comparison with Other PTM Prediction Models.

To further quantitatively evaluate the cDL-PTM models, the performance of the cDL-PTM models were compared to state of the art MusiteDeep, DeepPhos, and PTMscape models, which are the well-known deep-learning models and SVM model (PTMscape) using protein sequence information in PTM prediction.

MusiteDeep is further described in Wang et al., MusiteDeep: a deep-learning based webserver for protein posttranslational modification site prediction and visualization. Nucleic Acids Res. 2020, 48 (W1), W140-W146, which is hereby incorporated by reference in its entirety. DeepPhos is further described in Luo, F. et al, DeepPhos: prediction of protein phosphorylation sites with deep learning. Bioinformatics. 2019, 35 (16), 2766-2773, which is hereby incorporated by reference in its entirety. PTMscape is further described in Nguyen, V.-N. et al., A New Scheme to Characterize and Identify Protein Ubiquitination Sites. IEEE/ACM Trans. Comput. Biol. Bioinf 2017, 14 (2), 393-403, which is hereby incorporated by reference in its entirety.

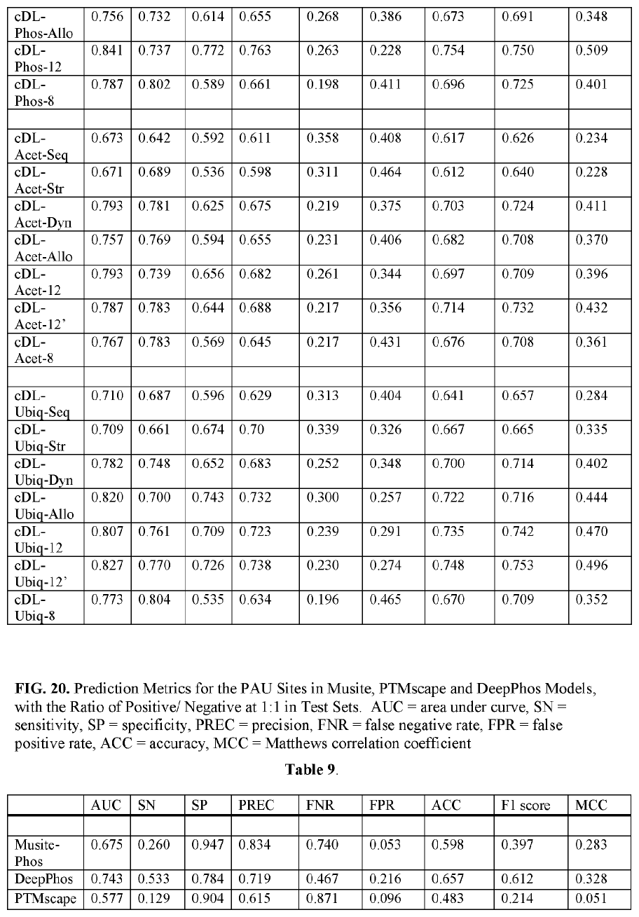

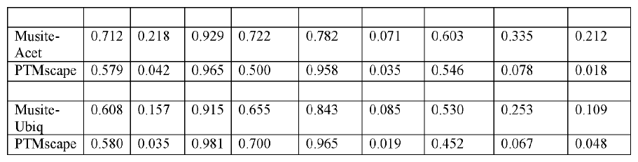

In the prediction tasks, the complete sequence information in the test set was used for evaluating these methods. As listed in FIG. 20, Table 9, DeepPhos achieves the highest AUC value of 0.743, and Musite-Acet reaches AUC value of 0.712. Notably, DeepPhos and Musite models outperform PTMscape models, indicating the advantage of deep learning methods in learning information from sequence data rather than traditional SVM models in PTMscape.

Compared with the proposed cDL-PAU, the prediction of PTM types in these state of the art models was not satisfying with dramatic performance loss. The overall better performance of the cDL-PAU models might be due to the following reason: in addition to the sequence information adopted by MusiteDeep, DeepPhos, and PTMscape, the cDL-PAU models considered multifaceted structural and dynamics features to characterize the PTM sites and fully utilized the complementarity among different signatures.

### REFERENCES

1. Beltrao, P.; Bork, P.; Krogan, N. J.; van Noort, V., Evolution and functional cross-talk of protein post-translational modifications. Molecular systems biology 2013, 9, 714.
2. Xu, H.; Wang, Y.; Lin, S.; Deng, W.; Peng, D.; Cui, Q.; Xue, Y., PTMD: A Database of Human Disease-associated Post-translational Modifications. Genomics Proteomics Bioinformatics 2018.
3. (a) Huang, K. Y.; Lee, T. Y.; Kao, H. J.; Ma, C. T.; Lee, C. C.; Lin, T. H.; Chang, W. C.; Huang, H. D., dbPTM in 2019: exploring disease association and cross-talk of post-translational modifications. Nucleic acids research 2019, 47 (D1), D298-D308; (b) Li, F.; Fan, C.; Marquez-Lago, T. T.; Leier, A.; Revote, J.; Jia, C.; Zhu, Y.; Smith, A. I.; Webb, G. I.; Liu, Q.; Wei, L.; Li, J.; Song, J., PRISMOID: a comprehensive 3D structure database for post-translational modifications and mutations with functional impact. Briefings in biomformatics 2020, 21 (3), 1069-1079; (c) Hornbeck, P. V.; Komhauser, J. M.; Latham, V.; Murray, B.; Nandhikonda, V.; Nord, A.; Skrzypek, E.; Wheeler, T.; Zhang, B.; Gnad, F., 15 years of PhosphoSitePlus®: integrating post-translationally modified sites, disease variants and isoforms. Nucleic acids research 2019, 47 (D1), D433-D441; (d) Craveur, P., PTM-SD: a database of structurally resolved and annotated posttranslational modifications in proteins. 2014.
4. (a) Zanzoni, A.; Ausiello, G.; Via, A.; Gherardini, P. F.; Helmer-Citterich, M., Phospho3D: a database of three-dimensional structures of protein phosphorylation sites. Nucleic acids research 2007, 35 (Database issue), D229-31; (b) Zanzoni, A.; Carbajo, D.; Diella, F.; Gherardini, P. F.; Tramontano, A.; Helmer-Citterich, M.; Via, A., Phospho3D 2.0: an enhanced database of three-dimensional structures of phosphorylation sites. Nucleic acids research 2011, 39 (Database issue), D268-71.
5. Su, M. G.; Huang, K. Y.; Lu, C. T.; Kao, H. J.; Chang, Y. H.; Lee, T. Y., topP™: a new module of dbPTM for identifying functional post-translational modifications in transmembrane proteins. Nucleic acids research 2014, 42 (Database issue), D537-45.
6. Smith, K. P.; Gifford, K. M.; Waitzman, J. S.; Rice, S. E., Survey of phosphorylation near drug binding sites in the Protein Data Bank (PDB) and their effects. Proteins 2015, 83 (1), 25-36.
7. Su, M. G.; Weng, J. T.; Hsu, J. B.; Huang, K. Y.; Chi, Y. H.; Lee, T. Y., Investigation and identification of functional post-translational modification sites associated with drug binding and protein-protein interactions. BMC Syst Biol 2017, 11 (Suppl 7), 132.
8. Xin, F.; Radivojac, P., Post-translational modifications induce significant yet not extreme changes to protein structure. Bionformatics 2012, 28 (22), 2905-13.
9. Craveur, P.; Narwani, T. J.; Rebehmed, J.; de Brevern, A. G., Investigation of the impact of PTMs on the protein backbone conformation. Amino Acids 2019, 51 (7), 1065-1079.
10. (a) Meng, Y; Roux, B., Locking the active conformation of c-Src kinase through the phosphorylation of the activation loop. Journal of molecular biology 2014, 426 (2), 423-35; (b) Shukla, D.; Meng, Y; Roux, B.; Pande, V. S., Activation pathway of Src kinase reveals intermediate states as targets for drug design. Nature communications 2014, 5, 3397; (c) Li, Y; Nam, K., Dynamic, structural and thermodynamic basis of insulin-like growth factor 1 kinase allostery mediated by activation loop phosphorylation. Chemical science 2017, 8 (5), 3453-3464.
11. Nussinov, R.; Tsai, C. J.; Xin, F.; Radivojac, P., Allosteric post-translational modification codes. Trends Biochem Sci 2012, 37 (10), 447-55.
12. Meng, F.; Liang, Z.; Zhao, K.; Luo, C., Drug design targeting active posttranslational modification protein isoforms. Medicinal research reviews 2020.
13. Steinberg, S. F., Post-translational modifications at the ATP-positioning G-loop that regulate protein kinase activity. Pharmacological research 2018, 135, 181-187.
14. Ishizawar, R.; Parsons, S. J., c-Src and cooperating partners in human cancer. Cancer cell 2004, 6 (3), 209-214.
15. (a) Okada, M.; Nakagawa, H., A Protein Tyrosine Kinase Involved in Regulation of pp60c-src function. Journal of Biological Chemistry 1989, 264 (35), 20886-20893; (b) Brown, M. T.; Cooper, J. A., Regulation, substrates and functions of src. Biochimica et Biophysica Acta (BBA)—Reviews on Cancer 1996, 1287 (2), 121-149; (c) Thomas, S. M.; Brugge, J. S., Cellular functions regulated by Src family kinase. Annual Review of Cell and Developmental Biology 1997, 13 (1), 513-609.
16. (a) Roskoski, R., Src protein-tyrosine kinase structure, mechanism, and small molecule inhibitors. Pharmacological research 2015, 94, 9-25; (b) Xu, W.; Doshi, A.; Lei, M.; Eck, M. J.; Harrison, S. C., Crystal structures of c-Src reveal features of its autoinhibitory mechanism. Molecular cell 1999, 3 (5), 629-638.
17. (a) Ingley, E., Src family kinases: Regulation of their activities, levels and identification of new pathways. Biochimica et Biophysica Acta (BBA)—Proteins and Proteomics 2008, 1784 (1), 56-65; (b) Simatou, A.; Simatos, G.; Goulielmaki, M.; Spandidos, D. A.; Baliou, S.; Zoumpourlis, V., Historical retrospective of the SRC oncogene and new perspectives Mol Clin Oncol 2020, 13 (4), 21-21.
18. (a) Irby, R. B.; Yeatman, T. J., Role of Src expression and activation in human cancer. Oncogene 2000, 19 (49), 5636-5642; (b) Biscardi, J. S.; Ishizawar, R. C.; Silva, C. M.; Parsons, S. J., Tyrosine kinase signalling in breast cancer: epidermal growth factor receptor and c-Src interactions in breast cancer. Breast Cancer Res 2000, 2 (3), 203-210; (c) Playford, M. P.; Schaller, M. D., The interplay between Src and integrins in normal and tumor biology. Oncogene 2004, 23 (48), 7928-7946.
19. Liang, Z.; Verkhivker, G. M.; Hu, G., Integration of network models and evolutionary analysis into high-throughput modeling of protein dynamics and allosteric regulation: theory, tools and applications. Briefings in bioinformatics 2019.
20. (a) Astl, L.; Verkhivker, G M., Data-driven computational analysis of allosteric proteins by exploring protein dynamics, residue coevolution and residue interaction networks. Biochim Biophys Acta Gen Subj 2019; (b) Yan, W.; Zhang, D.; Shen, C.; Liang, Z.; Hu, G., Recent Advances on the Network Models in Target-based Drug Discovery. Current topics in medicinal chemistry 2018, 18 (13), 1031-1043.
21. (a) Ponzoni, L.; Bahar, I., Structural dynamics is a determinant of the functional significance of missense variants. Proceedings of the National Academy of Sciences of the United States of America 2018, 115 (16), 4164-4169; (b) Yan, W.; Hu, G.; Liang, Z.; Zhou, J.; Yang, Y.; Chen, J.; Shen, B., Node-Weighted Amino Acid Network Strategy for Characterization and Identification of Protein Functional Residues. Journal of chemical information and modeling 2018, 58 (9), 2024-2032.
22. (a) Csermely, P.; Korcsmaros, T.; Kiss, H. J.; London, G.; Nussinov, R., Structure and dynamics of molecular networks: a novel paradigm of drug discovery: a comprehensive review. Pharmacol Ther 2013, 138 (3), 333-408; (b) Mishra, S. K.; Kandoi, G.; Jernigan, R. L., Coupling dynamics and evolutionary information with structure to identify protein regulatory and functional binding sites. Proteins 2019, 87 (10), 850-868; (c) Ma, X.; Meng, H.; Lai, L., Motions ofAllosteric and Orthosteric Ligand-Binding Sites in Proteins are Highly Correlated. Journal of chemical information and modeling 2016, 56 (9), 1725-1733.
23. (a) Timmons, S.; Coakley, M. F.; Moloney, A. M.; C, O. N., Akt signal transduction dysfunction in Parkinson's disease. Neuroscience letters 2009, 467 (1), 30-5; (b) Barragan, M.; de Frias, M.; Iglesias-Serret, D.; Campas, C.; Castano, E.; Santidrian, A. F.; Coll-Mulet, L.; Cosialls, A. M.; Domingo, A.; Pons, G.; Gil, J., Regulation of Akt/PKB by phosphatidylinositol 3-kinase-dependent and -independent pathways in B-cell chronic lymphocytic leukemia cells: role of protein kinase C{beta}. Journal of leukocyte biology 2006, 80 (6), 1473-9; (c) Ho, L.; Qin, W.; Pompl, P. N.; Xiang, Z.; Wang, J.; Zhao, Z.; Peng, Y.; Cambareri, G.; Rocher, A.; Mobbs, C. V.; Hof, P. R.; Pasinetti, G. M., Diet-induced insulin resistance promotes amyloidosis in atransgenic mouse model of Alzheimer's disease. FASEB journal: official publication of the Federation of American Societies for Experimental Biology 2004, 18 (7), 902-4; (d) Karlsson, H. K.; Zierath, J. R.; Kane, S.; Krook, A.; Lienhard, G. E.; Wallberg-Henriksson, H., Insulin-stimulated phosphorylation of the Akt substrate AS160 is impaired in skeletal muscle of type 2 diabetic subjects. Diabetes 2005, 54 (6), 1692-7; (e) Liu, T.; Fang, Y.; Zhang, H.; Deng, M.; Gao, B.; Niu, N.; Yu, J.; Lee, S.; Kim, J.; Qin, B.; Xie, F.; Evans, D.; Wang, L.; Lou, W.; Lou, Z., HEATRI Negatively Regulates Akt to Help Sensitize Pancreatic Cancer Cells to Chemotherapy. Cancer research 2016, 76 (3), 572-81.
24. Oliver, A. W.; Paul, A.; Boxall, K. J.; Barrie, S. E.; Aherne, G. W.; Garrett, M. D.; Mittnacht, S.; Pearl, L. H., Trans-activation of the DNA-damage signalling protein kinase Chk2 by T-loop exchange. The EMBO journal 2006, 25 (13), 3179-90.
25. Xie, T.; Peng, W.; Liu, Y.; Yan, C.; Maki, J.; Degterev, A.; Yuan, J.; Shi, Y., Structural basis of RIP1 inhibition by necrostatins. Structure 2013, 21 (3), 493-9.
26. Beltrao, P.; Albanese, V.; Kenner, L. R.; Swaney, D. L.; Burlingame, A.; Villen, J.; Lim, W. A.; Fraser, J. S.; Frydman, J.; Krogan, N. J., Systematic functional prioritization of protein posttranslational modifications. Cell 2012, 150 (2), 413-25.
27. Astl, L., Verkhivker, G. M., Dynamic View ofAllosteric Regulation in the Hsp70 Chaperones by J-Domain Cochaperone and Post-Translational Modifications: Computational Analysis of Hsp70 Mechanisms by Exploring Conformational Landscapes and Residue Interaction Networks. J Chem Inf Model 2020.
28. Mao, W.; Kaya, C.; Dutta, A.; Horovitz, A.; Bahar, I., Comparative study of the effectiveness and limitations of current methods for detecting sequence coevolution. Bioinformatics 2015, 31 (12), 1929-37.
29. (a) Stetz, G.; Verkhivker, G. M., Computational Analysis of Residue Interaction Networks and Coevolutionary Relationships in the Hsp70 Chaperones: A Community-Hopping Model of Allosteric Regulation and Communication. PLoS ComputBiol 2017, 13 (1), e1005299; (b) Amusengeri, A.; Astl, L.; Lobb, K.; Verkhivker, G. M.; Tastan Bishop, O., Establishing Computational Approaches Towards Identifying Malarial Allosteric Modulators: A Case Study of Plasmodium falciparum Hsp70s. International journal of molecular sciences 2019, 20 (22).
30. Liu, H.-F. L. a. R., Structure-based prediction of post-translational modification cross-talk within proteins using complementary residue- and residue pair-based features. Briefings in bioinformatics 2019.
31. Wood, D. J.; Lopez-Fernandez, J. D.; Knight, L. E.; Al-Khawaldeh, I.; Gai, C.; Lin, S.; Martin, M. P.; Miller, D. C.; Cano, C.; Endicott, J. A.; Hardcastle, I. R.; Noble, M. E. M.; Waring, M. J., FragLites-Minimal, Halogenated Fragments Displaying Pharmacophore Doublets. An Efficient Approach to Druggability Assessment and Hit Generation. Journal of medicinal chemistry 2019, 62 (7), 3741-3752.
32. Betzi, S.; Alam, R.; Martin, M.; Lubbers, D. J.; Han, H.; Jakkaraj, S. R.; Georg, G. I.; Schonbrunn, E., Discovery of a potential allosteric ligand binding site in CDK2. ACS chemical biology 2011, 6 (5), 492-501.
33. Schoepfer, J.; Jahnke, W.; Berellini, G.; Buonamici, S.; Cotesta, S.; Cowan-Jacob, S. W.; Dodd, S.; Drueckes, P.; Fabbro, D.; Gabriel, T.; Groell, J. M.; Grotzfeld, R. M.; Hassan, A. Q.; Henry, C.; Iyer, V.; Jones, D.; Lombardo, F.; Loo, A.; Manley, P. W.; Pelle, X.; Rummel, G.; Salem, B.; Warmuth, M.; Wylie, A. A.; Zoller, T.; Marzinzik, A. L.; Furet, P., Discovery of Asciminib (ABL001), an Allosteric Inhibitor of the Tyrosine Kinase Activity of BCR-ABL1. Journal of medicinal chemistry 2018, 61 (18), 8120-8135.
34. Wylie, A. A.; Schoepfer, J.; Jahnke, W.; Cowan-Jacob, S. W.; Loo, A.; Furet, P.; Marzinzik, A. L.; Pelle, X.; Donovan, J.; Zhu, W.; Buonamici, S.; Hassan, A. Q.; Lombardo, F.; Iyer, V; Palmer, M.; Berellini, G.; Dodd, S.; Thohan, S.; Bitter, H.; Branford, S.; Ross, D. M.; Hughes, T P.; Petruzzelli, L.; Vanasse, K. G.; Warmuth, M.; Hofinann, F.; Keen, N. J.; Sellers, W. R., The allosteric inhibitor ABL001 enables dual targeting of BCR-ABL1. Nature 2017, 543 (7647), 733-737.
35. Capra, J. A.; Laskowski, R. A.; Thornton, J. M.; Singh, M.; Funkhouser, T. A., Predicting protein ligand binding sites by combining evolutionary sequence conservation and 3D structure. PLoS computational biology 2009, 5 (12), e1000585.
36. (a) Fajer, M.; Meng, Y.; Roux, B., The Activation of c-Src Tyrosine Kinase: Conformational Transition Pathway and Free Energy Landscape. The journal of physical chemistry. B 2017, 121 (15), 3352-3363; (b) Meng, Y.; Shukla, D.; Pande, V. S.; Roux, B., Transition path theory analysis of c-Src kinase activation. Proceedings of the National Academy of Sciences of the United States of America 2016, 113 (33), 9193-8; (c) Pucheta-Martinez, E.; Saladino, G.; Morando, M. A.; Martinez-Torrecuadrada, J.; Lelli, M.; Sutto, L.; D'Amelio, N.; Gervasio, F. L., An Allosteric Cross-Talk Between the Activation Loop and the ATP Binding Site Regulates the Activation of Src Kinase. Scientific reports 2016, 6, 24235; (d) Meng, Y.; Pond, M. P.; Roux, B., Tyrosine Kinase Activation and Conformational Flexibility: Lessons from Src-Family Tyrosine Kinases. Accounts of chemical research 2017, 50 (5), 1193-1201; (e) Wenging Xu, A. D., Ming Lei, Michael J. Eck, and Stephen C. Harrison, Crystal Structures of c-Src Reveal Features of Its Autoinhibitory Mechanism. Molecular cell 1999, 3, 629-638.
37. Heppner, D. E.; Dustin, C. M.; Liao, C.; Hristova, M.; Veith, C.; Little, A. C.; Ahlers, B. A.; White, S. L.; Deng, B.; Lam, Y. W.; Li, J.; van der Vliet, A., Direct cysteine sulfenylation drives activation of the Src kinase. Nature communications 2018, 9 (1), 4522.
38. Cowan-Jacob, S. W.; Fendrich, G.; Manley, P. W.; Jahnke, W.; Fabbro, D.; Liebetanz, J.; Meyer, T., The crystal structure of a c-Src complex in an active conformation suggests possible steps in c-Src activation. Structure 2005, 13 (6), 861-71.
39. (a) Du, G.; Rao, S.; Gurbani, D.; Henning, N. J.; Jiang, J.; Che, J.; Yang, A.; Ficarro, S. B.; Marto, J. A.; Aguirre, A. J.; Sorger, P. K.; Westover, K. D.; Zhang, T.; Gray, N. S., Structure-Based Design of a Potent and Selective Covalent Inhibitor for SRC Kinase That Targets a P-Loop Cysteine. Journal of medicinal chemistry 2020, 63 (4), 1624-1641; (b) Kwarcinski, F. E.; Fox, C. C.; Steffey, M. E.; Soellner, M. B., Irreversible inhibitors of c-Src kinase that target a nonconserved cysteine. ACS chemical biology 2012, 7 (11), 1910-7.
40. Roskoski, R., Jr., Src protein-tyrosine kinase structure, mechanism, and small molecule inhibitors. Pharmacological research 2015, 94, 9-25.
41. Bakan, A.; Bahar, I., The intrinsic dynamics of enzymes plays a dominant role in determining the structural changes induced upon inhibitor binding. Proceedings of the National Academy of Sciences of the United States of America 2009, 106 (34), 14349-54.
42. Sievers, F.; Higgins, D. G., Clustal Omega for making accurate alignments of many protein sequences. Protein science: a publication of the Protein Society 2018, 27 (1), 135-145.
43. Liu, Y.; Bahar, I., Sequence evolution correlates with structural dynamics. Molecular biology and evolution 2012, 29 (9), 2253-63.
44. Wolfgang Kabsch, C. S., Dictionary of Protein Secondary Structure: Pattern Recognition of Hydrogen-Bonded and Geometrical Features. Biophysical journal 1983, 22, 2577-2637.
45. Le Guilloux, V.; Schmidtke, P.; Tuffery, P., Fpocket. an open source platform for ligand pocket detection. BMC bioinformatics 2009, 10, 168.
46. Grant, B. J.; Skjaerven, L.; Yao, X. Q., The Bio3D packages for structural bioinfonnatics. Protein science: a publication of the Protein Society 2021, 30 (1), 20-30.
47. Zhang, S.; Krieger, J. M.; Zhang, Y.; Kaya, C.; Kaynak, B.; Mikulska-Ruminska, K.; Doruker, P.; Li, H.; Bahar, I., ProDy 2.0: Increased Scale and Scope after 10 Years of Protein Dynamics Modelling with Python. Bioinformatics 2021.
48. General, I. J.; Liu, Y.; Blackburn, M. E.; Mao, W.; Gierasch, L. M.; Bahar, I., ATPase Subdomain IA Is a Mediator of Interdomain Allostery in Hsp70 Molecular Chaperones. PLoS Computational Biology 2014, 10 (5), e1003624.
49. Zhu, K.; Borrelli, K. W.; Greenwood, J. R.; Day, T.; Abel, R.; Farid, R. S.; Harder, E., Docking covalent inhibitors: a parameter free approach to pose prediction and scoring. Journal of chemical information and modeling 2014, 54 (7), 1932-40.
50. Xu, W.; Harrison, S. C.; Eck, M. J., Three-dimensional structure of the tyrosine kinase c-Src. Nature 1997, 385 (6617), 595-602.

### INCORPORATION BY REFERENCE

References and citations to other documents, such as patents, patent applications, patent publications, journals, books, papers, web contents, have been made in this disclosure. All such documents are hereby incorporated herein by reference in their entirety for all purposes. Any material, or portion thereof, that is said to be incorporated by reference herein, but which conflicts with existing definitions, statements, or other disclosure material explicitly set forth herein is only incorporated to the extent that no conflict arises between that incorporated material and the present disclosure material. In the event of a conflict, the conflict is to be resolved in favor of the present disclosure as the preferred disclosure.

### EQUIVALENTS

The representative examples are intended to help illustrate the invention, and are not intended to, nor should they be construed to, limit the scope of the invention. Indeed, various modifications of the invention and many further embodiments thereof, in addition to those shown and described herein, will become apparent to those skilled in the art from the full contents of this document, including the examples and the references to the scientific and patent literature included herein. The examples contain additional information, exemplification and guidance that can be adapted to the practice of this invention in its various embodiments and equivalents thereof.

1. A method for performing screening of pharmacophores or compounds for an allosteric interaction with a site of a protein, the method comprising:
categorizing PTM features of a site of the protein into sequence features (SEQ), structural and topological features (STR), and/or dynamic features (DYN);
applying a machine learning model to analyze the SEQ, STR, and/or DYN features, the machine learning model trained to classify the site of the protein as an allosteric PTM pocket or a non-allosteric PTM pocket;
responsive to the classification of the site of the protein as an allosteric PTM pocket, applying a pharmacophore or a compound to the allosteric pocket via molecular modeling to determine a level of allosteric interaction between the pharmacophore or compound and the protein.

2. The method of claim 1, wherein the sequence features (SEQ), structural and topological features (STR), and/or dynamic features (DYN) comprise each of sequence features (SEQ), structural and topological features (STR), and dynamic features (DYN), and wherein applying the machine learning model comprises applying the machine learning model to analyze the SEQ, STR and DYN features.

3. The method of claim 1, wherein the molecular modeling comprises covalent docking of the pharmacophore or compound to the allosteric PTM pocket.

4. The method of claim 1, wherein the molecular modeling comprises non-covalent docking of the pharmacophore or compound to the allosteric PTM pocket.

5. The method of claim 1, wherein categorizing PTM features comprises protein modeling.

6. The method of claim 5, wherein the protein modeling comprises anisotropic network model (ANM) analysis or Gaussian network model (GNM) analysis.

7. (canceled)

8. The method of claim 5, wherein the protein modeling comprises principal component analysis (PCA) analysis.

9. The method of claim 1, wherein the machine learning model comprises a random forest (RF) model or a fully connected neural network (FCNN) model.

10. (canceled)

11. The method of claim 1, wherein the protein is an enzyme.

12. The method of claim 11, wherein the enzyme is a kinase.

13. The method of claim 12, wherein the kinase is of a family selected from the group consisting of: cyclin-dependent kinases (CDKs), Protein kinase B (AKTs), nonreceptor tyrosine kinases (NRTK), p21-activated kinases (PAKs), checkpoint kinases (CHKs), and receptor-interacting protein (RIP) kinases.

14. The method of claim 1, wherein the PTM is of a type selected from the group consisting of: phosphorylation, glycosylation, ubiquitylation, acetylation, sumoylation, glutathionylation, methylation, succinylation, and S-nitrosylation.

15. The method of claim 14, wherein the PTM is phosphorylation.

16. The method of claim 1, wherein the pharmacophore or compound is a de novo pharmacophore or compound.

17. The method of claim 1, wherein the pharmacophore or compound is a known pharmacophore or compound.

18. The method of claim 1, wherein all steps are performed in silico.

19. The method of claim 1, further comprising: performing a microscopic analysis, crystal structural analysis, and/or a biophysical assay to determine the level of allosteric interaction.

20. The method of claim 1, further comprising: performing an in vitro and/or in vivo biological assay to confirm the level of allosteric interaction.

21. The method of claim 1, further comprising: optimizing the de novo pharmacophore or compound to modify the interaction between the pharmacophore or compound with the protein, or to modify off-target effects of the pharmacophore or compound.

22. A system or an apparatus comprising a non-transitory computer-readable memory, a processor and a communication interface wherein the processor is connected to the non-transitory computer-readable memory and the communication interface, wherein the processor is adapted to execute instructions stored on the non-transitory computer readable memory such that, when executed, cause the processor to perform or implement a method according to claim 1.

23. A pharmacophore or compound identified by the method of claim 1.

24-44. (canceled)
